SEQUENCE LISTING (74 sequences)

SEQ ID NO: 1 - DNA, 86 bp, Artificial Sequence
  Synthetic Sequence
atatatatgggaggacgatgcgggcacatcactctatcatttgctacggtaccggagtgagtccagacgacgagcgggaaaaaaaa

SEQ ID NO: 2 - DNA, 76 bp, Artificial Sequence
  Synthetic Sequence
atatatatgggaggacgatgcggcaacccaccactctatctttcccataactgcagacgacgagcgggaaaaaaaa

SEQ ID NO: 3 - DNA, 76 bp, Artificial Sequence
  Synthetic Sequence
atatatatgggaggacgatgcgggccaatctgtcttctttccatccttatgatcagacgacgagcgggaaaaaaaa

SEQ ID NO: 4 - DNA, 76 bp, Artificial Sequence
  Synthetic Sequence
atatatatgggaggacgatgcggcaaccacacgcaggaggacacaacgatccgcagacgacgagcgggaaaaaaaa

SEQ ID NO: 5 - DNA, 76 bp, Artificial Sequence
  Synthetic Sequence
atatatatgggaggacgatgcgggacgagggaccagaccgccacagcgggatgcagacgacgagcgggaaaaaaaa

SEQ ID NO: 6 - DNA, 76 bp, Artificial Sequence
  Synthetic Sequence
atatatatgggaggacgatgcgggcggaagaggcagggtaccacggcagaggtcagacgacgagcgggaaaaaaaa

SEQ ID NO: 7 - DNA, 76 bp, Artificial Sequence
  Synthetic Sequence
atatatatgggaggacgatgcgggcgaaggcacaccgagttcatagtatcccacagacgacgagcgggaaaaaaaa

SEQ ID NO: 8 - DNA, 76 bp, Artificial Sequence
  Synthetic Sequence
atatatatgggaggacgatgcgggccaacccctagtgaacaacaacactcccacagacgacgagcgggaaaaaaaa

SEQ ID NO: 9 - DNA, 73 bp, Artificial Sequence
  Synthetic Sequence
atatatatgggaggacgatgcgggcagtaggttgggtagggtggtctgctcagacgacgagcgggaaaaaaaa

SEQ ID NO: 10 - DNA, 73 bp, Artificial Sequence
  Synthetic Sequence
atatatatgggaggacgatgcgggaggagctgatgggtggtgaggttggccagacgacgagcgggaaaaaaaa

SEQ ID NO: 11 - DNA, 76 bp, Artificial Sequence
  Synthetic Sequence
atatatatgggaggacgatgcgggcaggacggacagcaaggggtgagcacgagcagacgacgagcgggaaaaaaaa

SEQ ID NO: 12 - DNA, 74 bp, Artificial Sequence
  Synthetic Sequence
atatatatgggaggacgatgcgggcggttggcgtggttggaaatgtcccgtcagacgacgagcgggaaaaaaaa

SEQ ID NO: 13 - DNA, 76 bp, Artificial Sequence
  Synthetic Sequence
atatatatgggaggacgatgcgggaggaccacgaccatgacccaccaggaatgcagacgacgagcgggaaaaaaaa

SEQ ID NO: 14 - DNA, 76 bp, Artificial Sequence
  Synthetic Sequence
atatatatgggaggacgatgcgggcacaggcctaacatacctccatctcctggcagacgacgagcgggaaaaaaaa

SEQ ID NO: 15 - DNA, 76 bp, Artificial Sequence
  Synthetic Sequence
atatatatgggaggacgatgcgggaccaacgagaccacacgacaagcgctgtgcagacgacgagcgggaaaaaaaa

SEQ ID NO: 16 - DNA, 73 bp, Artificial Sequence
  Synthetic Sequence
atatatatgggaggacgatgcgggccatggatggtttggttggctgtcctcagacgacgagcgggaaaaaaaa

SEQ ID NO: 17 - DNA, 76 bp, Artificial Sequence
  Synthetic Sequence
atatatatgggaggacgatgcggcagcaccgaggtacccaacagggatccgcccagacgacgagcgggaaaaaaaa

SEQ ID NO: 18 - DNA, 76 bp, Artificial Sequence
  Synthetic Sequence
atatatatgggaggacgatgcgggcggcagacgcgccgggtaccccaggtccccagacgacgagcgggaaaaaaaa

SEQ ID NO: 19 - DNA, 76 bp, Artificial Sequence
  Synthetic Sequence
atatatatgggaggacgatgcggcacaaggaacaaagcggcccctatccccaacagacgacgagcgggaaaaaaaa

SEQ ID NO: 20 - DNA, 76 bp, Artificial Sequence
  Synthetic Sequence
atatatatgggaggacgatgcggggggcaagaagcacggtaccccaggtccgccagacgacgagcgggaaaaaaaa

SEQ ID NO: 21 - DNA, 76 bp, Artificial Sequence
  Synthetic Sequence
atatatatgggaggacgatgcggccggacatcccccagggcaaaaccaactcccagacgacgagcgggaaaaaaaa

SEQ ID NO: 22 - DNA, 76 bp, Artificial Sequence
  Synthetic Sequence
atatatatgggaggacgatgcggcaagggaaacagatagcccaggctcccccccagacgacgagcgggaaaaaaaa

SEQ ID NO: 23 - DNA, 86 bp, Artificial Sequence
  Synthetic Sequence
atatatatgggaggacgatgcgggccaaccacgtggtattattgaccttgcaatgggaatgcccagacgacgagcgggaaaaaaaa

SEQ ID NO: 24 - DNA, 86 bp, Artificial Sequence
  Synthetic Sequence
atatatatgggaggacgatgcggggcaaactgcgtcgtattataagcctcgctacagatgccacagacgacgagcgggaaaaaaaa

SEQ ID NO: 25 - DNA, 86 bp, Artificial Sequence
  Synthetic Sequence
atatatatgggaggacgatgcgggcacctacctgagctacatatgacagtgtcaccctggccccagacgacgagcgggaaaaaaaa

SEQ ID NO: 26 - DNA, 86 bp, Artificial Sequence
  Synthetic Sequence
atatatatgggaggacgatgcgggccaaatggacttttcgccacgaacttacgacggtgttgccagacgacgagcgggaaaaaaaa

SEQ ID NO: 27 - DNA, 86 bp, Artificial Sequence
  Synthetic Sequence
atatatatgggaggacgatgcggcaccaaaaggtggtcttagcctaattatggacgtgtccaccagacgacgagcgggaaaaaaaa

SEQ ID NO: 28 - DNA, 86 bp, Artificial Sequence
  Synthetic Sequence
atatatatgggaggacgatgcgggccacgtgtattatcctcagcttatagccatggcatggaccagacgacgagcgggaaaaaaaa

SEQ ID NO: 29 - DNA, 86 bp, Artificial Sequence
  Synthetic Sequence
atatatatgggaggacgatgcgggcaaagtcttggtccaccaaatatgtgatgtcaccaccagcagacgacgagcgggaaaaaaaa

SEQ ID NO: 30 - DNA, 86 bp, Artificial Sequence
  Synthetic Sequence
atatatatgggaggacgatgcgggccctacttgcatgaatatccactcctaggcttgagggagcagacgacgagcgggaaaaaaaa

SEQ ID NO: 31 - DNA, 86 bp, Artificial Sequence
  Synthetic Sequence
atatatatgggaggacgatgcgggccgaagtctaaacctgctcgtgactttctttcgatgttgcagacgacgagcgggaaaaaaaa

SEQ ID NO: 32 - DNA, 86 bp, Artificial Sequence
  Synthetic Sequence
atatatatgggaggacgatgcgggcctaccaactcccctctagtcctgttctatccacgttggcagacgacgagcgggaaaaaaaa

SEQ ID NO: 33 - DNA, 86 bp, Artificial Sequence
  Synthetic Sequence
atatatatgggaggacgatgcgggccaaggttcccttctgcctcattgttgtgggaacccatccagacgacgagcgggaaaaaaaa

SEQ ID NO: 34 - DNA, 86 bp, Artificial Sequence
  Synthetic Sequence
atatatatgggaggacgatgcggccccgagtttccctaaggtttggttgacctgtcatttcagcagacgacgagcgggaaaaaaaa

SEQ ID NO: 35 - DNA, 85 bp, Artificial Sequence
  Synthetic Sequence
atatatatgggaggacgatgcgggcacaggttctatcaacgttgtcctgagtaattgacctgcagacgacgagcgggaaaaaaaa

SEQ ID NO: 36 - DNA, 86 bp, Artificial Sequence
  Synthetic Sequence
atatatatgggaggacgatgcgggccaaggacattcttgttcgttgttgctgtccactgtctccagacgacgagcgggaaaaaaaa

SEQ ID NO: 37 - DNA, 86 bp, Artificial Sequence
  Synthetic Sequence
atatatatgggaggacgatgcgggaccaataacactacactgatcatctcccttctatgtccccagacgacgagcgggaaaaaaaa

SEQ ID NO: 38 - DNA, 86 bp, Artificial Sequence
  Synthetic Sequence
atatatatgggaggacgatgcgggcacacttaaatccacttcaccttacaattcctttatctgcagacgacgagcgggaaaaaaaa

SEQ ID NO: 39 - DNA, 86 bp, Artificial Sequence
  Synthetic Sequence
atatatatgggaggacgatgcggccatacgcacttcagtggggataatccaactggtttggtgcagacgacgagcgggaaaaaaaa

SEQ ID NO: 40 - DNA, 86 bp, Artificial Sequence
  Synthetic Sequence
atatatatgggaggacgatgcgggaccaaataccaacttcacatcacctttcttattctccggcagacgacgagcgggaaaaaaaa

SEQ ID NO: 41 - DNA, 86 bp, Artificial Sequence
  Synthetic Sequence
atatatatgggaggacgatgcgggcactaactttacctccacctctaaccaccctcctttctgcagacgacgagcgggaaaaaaaa

SEQ ID NO: 42 - DNA, 86 bp, Artificial Sequence
  Synthetic Sequence
atatatatgggaggacgatgcgggccccaaacacttgttcctatctttcaaccccccttgatccagacgacgagcgggaaaaaaaa

SEQ ID NO: 43 - DNA, 86 bp, Artificial Sequence
  Synthetic Sequence
atatatatgggaggacgatgcggcgccccgattgaccttcgatttatcctacttatggcaccccagacgacgagcgggaaaaaaaa

SEQ ID NO: 44 - DNA, 86 bp, Artificial Sequence
  Synthetic Sequence
atatatatgggaggacgatgcggccatgaacccatcctctggttcataatcgacgtgttcgtgcagacgacgagcgggaaaaaaaa

SEQ ID NO: 45 - DNA, 85 bp, Artificial Sequence
  Synthetic Sequence
atatatatgggaggacgatgcggcacgagggaatcacctcgaacttgtcctggattactgcccagacgacgagcgggaaaaaaaa

SEQ ID NO: 46 - DNA, 86 bp, Artificial Sequence
  Synthetic Sequence
atatatatgggaggacgatgcgggctcaataacctgaatctacctttccctagcaaaggtctgcagacgacgagcgggaaaaaaaa

SEQ ID NO: 47 - DNA, 86 bp, Artificial Sequence; identical to SEQ ID NO: 39
  Synthetic Sequence

SEQ ID NO: 48 - DNA, 86 bp, Artificial Sequence
  Synthetic Sequence
atatatatgggaggacgatgcgggcacaaccttaccaccctagcctacccctaacctcctgtccagacgacgagcgggaaaaaaaa

SEQ ID NO: 49 - DNA, 86 bp, Artificial Sequence
  Synthetic Sequence
atatatatgggaggacgatgcgggaccatccaataccttccgtaacactttccttcttccttccagacgacgagcgggaaaaaaaa

SEQ ID NO: 50 - DNA, 86 bp, Artificial Sequence
  Synthetic Sequence
atatatatgggaggacgatgcgggcagcaacctaccttaccttcccctagcctaccttatccccagacgacgagcgggaaaaaaaa

SEQ ID NO: 51 - DNA, 86 bp, Artificial Sequence
  Synthetic Sequence
atatatatgggaggacgatgcgggcacctttcttacatcttggcttcattcttgcaccattggcagacgacgagcgggaaaaaaaa

SEQ ID NO: 52 - DNA, 86 bp, Artificial Sequence
  Synthetic Sequence
atatatatgggaggacgatgcgggcacaatcaagacctctccaaacttgaactctgtctatcccagacgacgagcgggaaaaaaaa

SEQ ID NO: 53 - DNA, 86 bp, Artificial Sequence
  Synthetic Sequence
atatatatgggaggacgatgcgggctgaaaggaaacggacgattgagcttccccttacctctccagacgacgagcgggaaaaaaaa

SEQ ID NO: 54 - DNA, 76 bp, Artificial Sequence
  Synthetic Sequence
atatatatgggaggacgatgcgggacgctagtaccctggctggcttggttgggcagacgacgagcgggaaaaaaaa

SEQ ID NO: 55 - DNA, 76 bp, Artificial Sequence
  Synthetic Sequence
atatatatgggaggacgatgcggccggttcacgtgcaccatccgtgtgctagacagacgacgagcgggaaaaaaaa

SEQ ID NO: 56 - DNA, 86 bp, Artificial Sequence
  Synthetic Sequence
atatatatgggaggacgatgcggcaaccctgacaccacgttgtttctccttttggggtaaccgcagacgacgagcgggaaaaaaaa

SEQ ID NO: 57 - DNA, 86 bp, Artificial Sequence
  Synthetic Sequence
atatatatgggaggacgatgcgggccgactctgaggaaaaggttttatgtatggctacccctgcagacgacgagcgggaaaaaaaa

SEQ ID NO: 58 - DNA, 86 bp, Artificial Sequence
  Synthetic Sequence
atatatatgggaggacgatgcgggcacacccaaccttgcttcttcaatctaatctccactttgcagacgacgagcgggaaaaaaaa

SEQ ID NO: 59 - DNA, 76 bp, Artificial Sequence
  Synthetic Sequence
atatatatgggaggacgatgcgggcgtctgggatttggacttcttcgctagctcagacgacgagcgggaaaaaaaa

SEQ ID NO: 60 - DNA, 76 bp, Artificial Sequence
  Synthetic Sequence
atatatatgggaggacgatgcggctgcgtgacagttatactgttattggtcttcagacgacgagcgggaaaaaaaa

SEQ ID NO: 61 - DNA, 76 bp, Artificial Sequence
  Synthetic Sequence
atatatatgggaggacgatgcggcacaatgaagtcactcttgacgcttgtattcagacgacgagcgggaaaaaaaa

SEQ ID NO: 62 - DNA, 76 bp, Artificial Sequence
  Synthetic Sequence
atatatatgggaggacgatgcggcctcataaagttacatcggcaattcttctccagacgacgagcgggaaaaaaaa

SEQ ID NO: 63 - DNA, 76 bp, Artificial Sequence
  Synthetic Sequence
atatatatgggaggacgatgcggctactcctccttaacccgggtcttgtggcccagacgacgagcgggaaaaaaaa

SEQ ID NO: 64 - DNA, 76 bp, Artificial Sequence
  Synthetic Sequence
atatatatgggaggacgatgcgggacgctaatacttctggagtggaacggtttcagacgacgagcgggaaaaaaaa

SEQ ID NO: 65 - DNA, 76 bp, Artificial Sequence
  Synthetic Sequence
atatatatgggaggacgatgcgggacgactagcctagtgcccttacgatcacccagacgacgagcgggaaaaaaaa

SEQ ID NO: 66 - DNA, 76 bp, Artificial Sequence
  Synthetic Sequence
atatatatgggaggacgatgcgggcaaagtgttatttcttgatctgtttcacccagacgacgagcgggaaaaaaaa

SEQ ID NO: 67 - DNA, 76 bp, Artificial Sequence
  Synthetic Sequence
atatatatgggaggacgatgcggcacctgatttctaccctttactttgtgtggcagacgacgagcgggaaaaaaaa

SEQ ID NO: 68 - DNA, 76 bp, Artificial Sequence
  Synthetic Sequence
atatatatgggaggacgatgcggcaccacttctttacctcactctttctgcagcagacgacgagcgggaaaaaaaa

SEQ ID NO: 69 - DNA, 76 bp, Artificial Sequence
  Synthetic Sequence
atatatatgggaggacgatgcgggccgactttgtcaccgagtgcatccgaggtcagacgacgagcgggaaaaaaaa

SEQ ID NO: 70 - DNA, 76 bp, Artificial Sequence
  Synthetic Sequence
atatatatgggaggacgatgcggnnnnnnnnnnnnnnnnnnnnnnnnnnnnnncagacgacgagcgggaaaaaaaa

SEQ ID NO: 71 - DNA, 78 bp, Artificial Sequence
  Synthetic Sequence
aattttttttcccgctcgtcgtctgnnnnnnnnnnnnnnnnnnnnnnnnnnnnnnccgcatcgtcctcccatatatat

SEQ ID NO: 72 - DNA, 23 bp, Artificial Sequence
  Synthetic Sequence
atatatatgggaggacgatgcgg

SEQ ID NO: 73 - DNA, 26 bp, Artificial Sequence
  Synthetic Sequence
aatttttttttcccgctcgtcgtctg

SEQ ID NO: 74 - DNA, 24 bp, Artificial Sequence
  Synthetic Sequence
tttttttttcccgctcgtcgtctg

(19) United States
(12) Patent Application Publication
(10) Pub. No.: US 2003/0054360 A1
(43) Pub. Date: Mar. 20, 2003

(54) Method and apparatus for the automated generation of nucleic acid ligands

(76) Inventors: Larry Gold, Boulder, CO (US); Dominic A. Zichi, Boulder, CO (US); Jonathan Drew Smith, Boulder, CO (US); Daniel J. Schneider, Arvada, CO (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 09/993,294

(22) Filed: Nov. 21, 2001

Related U.S. Application Data
(63) Continuation-in-part of application No. 09/815,171, filed on Mar. 22, 2001.
Application No. 09/815,171 is a continuation-in-part of application No. 09/616,284, filed on Jul. 14, 2000.
Application No. 09/616,284 is a continuation-in-part of application No. 09/356,233, filed on Jul. 16, 1999.
Application No. 09/356,233 is a continuation-in-part of application No. 09/232,946, filed on Jan. 19, 1999.
(60) Provisional application No. 60/278,354, filed on Mar. 22, 2001.

(51) Int. Cl. C12Q 1/68; C12P 19/34

(52) U.S. Cl. 435/6; 435/91.2

(57) ABSTRACT

The present invention includes a method and device for performing the automated SELEX process, including automated photoSELEX process embodiments, and automated affinity SELEX process embodiments. The automated photoSELEX embodiments included an embodiment wherein target protein and nucleic acid ligands are photocrosslinked in solution. The steps of the SELEX process are performed at one or more work stations on a work surface by a robotic manipulator controlled by a computer. Also included in the invention are photocrosslinking nucleic acid ligands to human neutrophil elastase (hNE), HIV-1_{MN} gp120, human L-selectin, human P-Selectin, human platelet-derived growth factor (PDGF), human alpha-thrombin, human basic fibroblast growth factor (bFGF), HIV-1_{MN} gp120, Angiogenin, Interleukin-4, β-Nerve Growth Factor (β-NGF), Tansforming Growth Factor β1, Interleukin-7, Kininogen, Plasmin, Serum Amyloid P, Thrombopoietin (Tpo), Coagulation Factor IX, Coagulation Factor XII, Endostatin, Factor II, Collagen, Cytotoxic T lymphocyte-associated protein-4 Fc (CTLA-4 Fc), Hepatocyte Growth Factor (HGF), Insulin-like growth factor binding protein-3 (IGFBP-3), UDP-glucuronosyl transferase (UGT) 1A1, UGT 1A10, and UGT 1A3.

### RELATED APPLICATIONS

This application claims priority to United States Provisional Patent Application Serial No. 60/278,354, filed Mar. 22, 2001, entitled “The PhotoSELEX Process: Photocrosslinking of Target in Solution.” This application is also a continuation-in-part application of U.S. patent application Ser. No. 09/815,171, filed Mar. 22, 2001, which is a continuation-in-part application of U.S. patent application Ser. No. 09/616,284, filed Jul. 14, 2000, which is a continuation-in-part application of U.S. patent application Ser. No.09/356,233, filed Jul. 16, 1999, which is a continuation-in-part application of U.S. patent application Ser. No. 09/232,946, filed Jan. 19, 1999, each of which is entitled “Method and Apparatus for the Automated Generation of Nucleic Acid Ligands.”

### FIELD OF THE INVENTION

This invention is directed to a method for the generation of nucleic acid ligands having specific functions against target molecules using the SELEX process. The methods described herein enable nucleic acid ligands to be generated in dramatically shorter times and with much less operator intervention than was previously possible using prior art techniques. The invention includes a device capable of generating nucleic acid ligands with little or no operator intervention. The invention also includes the sequences of photocrosslinking nucleic acid ligands to protein targets generated using the described automated methods.

### BACKGROUND OF THE INVENTION

The dogma for many years was that nucleic acids had primarily an informational role. Through a method known as Systematic Evolution of Ligands by EXponential enrichment, termed the SELEX process, it has become clear that nucleic acids have three dimensional structural diversity not unlike proteins. The SELEX process is a method for the in vitro evolution of nucleic acid molecules with highly specific binding to target molecules and is described in U.S. patent application Ser. No. 07/536,428, filed Jun. 11, 1990, entitled “Systematic Evolution of Ligands by EXponential Enrichment,” now abandoned, U.S. Pat. No. 5,475,096 entitled “Nucleic Acid Ligands”, and U.S. Pat. No. 5,270,163 (see also WO 91/19813) entitled “Nucleic Acid Ligands” each of which is specifically incorporated by reference herein. Each of these patents and applications, collectively referred to herein as the SELEX Patent Applications, describes a fundamentally novel method for making a nucleic acid ligand to any desired target molecule. The SELEX process provides a class of products which are referred to as nucleic acid ligands or aptamers, each having a unique sequence, and which has the property of binding specifically to a desired target compound or molecule. Each SELEX process-identified nucleic acid ligand is a specific ligand of a given target compound or molecule.

The SELEX process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets. The SELEX process applied to the application of high affinity binding involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX process includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity nucleic acid ligands to the target molecule.

It has been recognized by the present inventors that the SELEX process demonstrates that nucleic acids as chemical compounds can form a wide array of shapes, sizes and configurations, and are capable of a far broader repertoire of binding and other functions than those displayed by nucleic acids in biological systems. The present inventors have recognized that SELEX or SELEX-like processes could be used to identify nucleic acids which can facilitate any chosen reaction in a manner similar to that in which nucleic acid ligands can be identified for any given target. In theory, within a candidate mixture of approximately 10¹³ to 10¹⁸ nucleic acids, the present inventors postulate that at least one nucleic acid exists with the appropriate shape to facilitate each of a broad variety of physical and chemical interactions.

The basic SELEX process has been modified to achieve a number of specific objectives. For example, U.S. patent application Ser. No. 07/960,093, filed Oct. 14, 1992, now abandoned, and U.S. Pat. No. 5,707,796, both entitled “Method for Selecting Nucleic Acids on the Basis of Structure,” describe the use of the SELEX process in conjunction with gel electrophoresis to select nucleic acid molecules with specific structural characteristics, such as bent DNA. U.S. Pat. No. 5,580,737 entitled “High-Affinity Nucleic Acid Ligands That Discriminate Between Theophylline and Caffeine,” describes a method for identifying highly specific nucleic acid ligands able to discriminate between closely related molecules, termed Counter-SELEX. U.S. Pat. No. 5,567,588 entitled “Systematic Evolution of Ligands by EXponential Enrichment: Solution SELEX,” describes a SELEX-based method which achieves highly efficient partitioning between oligonucleotides having high and low affinity for a target molecule. U.S. Pat. No. 5,496,938 entitled “Nucleic Acid Ligands to HIV-RT and HIV-1 Rev,” describes methods for obtaining improved nucleic acid ligands after SELEX has been performed. U.S. Pat. No. 5,705,337 entitled “Systematic Evolution of Ligands by Exponential Enrichment: Chemi-SELEX,” describes methods for covalently linking a ligand to its target.

The SELEX process encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX process-identified nucleic acid ligands containing modified nucleotides are described in U.S. Pat. No. 5,660,985 entitled “High Affinity Nucleic Acid Ligands Containing Modified Nucleotides,” that describes oligonucleotides containing nucleotide derivatives chemically modified at the 5- and 2′-positions of pyrimidines. U.S. Pat. No. 5,580,737, supra, describes highly specific nucleic acid ligands containing one or more nucleotides modified with 2′-amino (2′-NH2), 2′-fluoro (2′-F), and/or 2′-O-methyl (2′-OMe).

The SELEX process encompasses combining selected oligonucleotides with other selected oligonucleotides and non-oligonucleotide functional units as described in U.S. Pat. No. 5,637,459 entitled “Systematic Evolution of Ligands by EXponential Enrichment: Chimeric SELEX,” and U.S. Pat. No. 5,683,867 entitled “Systematic Evolution of Ligands by EXponential Enrichment: Blended SELEX,” respectively. These applications allow the combination of the broad array of shapes and other properties, and the efficient amplification and replication properties, of oligonucleotides with the desirable properties of other molecules.

The SELEX process further encompasses combining selected nucleic acid ligands with lipophilic compounds or non-immunogenic, high molecular weight compounds in a diagnostic or therapeutic complex as described in U.S. Pat. No. 6,011,020 entitled “Nucleic Acid Ligand Complexes.”

One potential problem encountered in the diagnostic use of nucleic acids is that oligonucleotides in their phosphodiester form may be quickly degraded in body fluids by intracellular and extracellular enzymes such as endonucleases and exonucleases before the desired effect is manifest. Certain chemical modifications of the nucleic acid ligand can be made to increase the in vivo stability of the nucleic acid ligand or to enhance or to mediate the delivery of the nucleic acid ligand. See, e.g., U.S. patent application Ser. No. 08/117,991, filed Sep. 9, 1993, now abandoned, and U.S. Pat. No. 5,660,985, both entitled “High Affinity Nucleic Acid Ligands Containing Modified Nucleotides”, and U.S. patent application Ser. No. 09/362,578 filed Jul. 28, 1999, entitled “Transcription-free SELEX”, each of which is specifically incorporated herein by reference. Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping. In preferred embodiments of the instant invention, the nucleic acid ligands are DNA molecules that are modified with a photoreactive group on 5-position of pyrimidine residues. The modifications can be pre- or post-SELEX process modifications.

The PhotoSELEX Process

One particularly important embodiment of the SELEX process is described in U.S. patent application Ser. No. 08/123,935, filed Sep. 17, 1993, and U.S. patent application Ser. No. 08/443,959 filed May 18, 1995, both entitled “Photoselection of Nucleic Acid Ligands,” and both now abandoned, and U.S. Pat. Nos. 5,763,177, 6,001,577, WO 95/08003, U.S. Pat. No. 6,291,184, U.S. patent application Ser. No. 09/619,213, filed Jul. 17, 2000, and U.S. patent application Ser. No. 09/723,718, filed Nov. 28, 2000, each of which is entitled “Systematic Evolution of Nucleic Acid Ligands by Exponential Enrichment: Photoselection of Nucleic Acid Ligands and Solution SELEX,” and each of which describe a SELEX process-based method for selecting nucleic acid ligands containing photoreactive groups capable of binding and/or photocrosslinking to and/or photoinactivating a target molecule. The resulting nucleic acid ligands are referred to as “photocrosslinking nucleic acid ligands” and “photoaptamers.” These patents and patent applications are referred to in this application collectively as “the PhotoSELEX Process Applications.” In the photoSELEX process embodiment of the SELEX process, a modified nucleotide activated by absorption of light is incorporated in place of a native base in either RNA- or in ssDNA-randomized oligonucleotide libraries. One such photoreactive nucleotide whose photochemistry is particularly well-suited for this purpose is 5-bromo-2′-deoxyuridine (5-BrdU) (Meisenheimer and Koch (1997) Crit. Rev. Biochem. Mol. Biol. 32:101-140). The 5-BrdU chromophore absorbs ultraviolet (UV) light in the 310 nm range where native chromophores of nucleic acids and proteins do not absorb or absorb very weakly. The resulting excited singlet state intersystem crosses to the lowest triplet state which specifically crosslinks with aromatic and sulfur-bearing amino acid residues of a protein target in suitable proximity (Dietz and Koch (1987) Photochem. Photobiol. 46:971-8; Dietz and Koch (1989) Photochem. Photobiol. 49:121-9; Dietz et al. (1987) J. Am. Chem. Soc. 109:1793-1797; Ito et al. (1980) J. Am. Chem. Soc. 102:7535-7541; Swanson et al. (1981) J. Am. Chem. Soc. 103:1274-1276). Crosslinking may also occur via excitation of an aromatic residue of the protein in proximity to the bromouracil chromophore (Norris et al. (1997) Photochem. Photobiol. 65:201-207). Of particular importance, excited bromouracil in DNA is relatively unreactive in the absence of a proximal, oriented, reactive amino acid (Gott et al. (1991) Biochemistry 30:6290-6295; Willis et al (1994) Nucleic Acids Res. 22:4947-4952; Norris et al. (1997) Photochem. Photobiol. 65:201-207) or nucleotide residue (Sugiyama et al. (1990) J. Am. Chem. Soc. 112:6720-6721; Cook and Greenberg (1996) J. Am. Chem. Soc. 118:10025-10030). The importance of orientation is evident in crystal structures of protein-nucleic acid complexes which show a lock and key arrangement of the bromouracil chromophore with the aromatic amino acid residue to which it crosslinks (Horvath et al. (1998) Cell 95:963-974; Meisenheimer and Koch (1997) Crit. Rev. Biochem. Mol. Biol. 32:101-140).

In a basic embodiment, the photoSELEX process comprises the following steps:

a) A candidate mixture of nucleic acids is prepared. The candidate mixture nucleic acids comprise sequences with randomized regions including photoreactive groups, e.g. by incorporating 5-BrdU into the candidate mixture.

b) The candidate mixture is contacted with a quantity of target. Nucleic acid ligands of the target in the candidate mixture form complexes with the target;

c) The photoreactive groups in candidate nucleic acid ligands are photoactivated by irradiation. Nucleic acid ligands that have formed specific complexes with target thereby become photocrosslinked to the target;

d) Nucleic acid ligands that have become photocrosslinked to target are partitioned from other nucleic acids in the candidate mixture;

e) The nucleic acid ligands that photocrosslinked to the target are released from the target (e.g., by protease digestion if the target is a protein), and then amplified; and

f) The amplified nucleic acid ligands are used as the candidate mixture to initiate another round of the photoSELEX process.

The photoSELEX process produces nucleic acid ligands which are single- or double-stranded RNA or DNA oligonucleotides. A photoreactive group may comprise a natural nucleic acid residue with a relatively simple modification that confers increased reactivity or photoreactivity to the nucleic acid residue. Such modifications include, but are not limited to, modifications at cytosine exocyclic amines, substitution with halogenated groups, e.g., 5′-bromo- or 5′-iodo-uracil, modification at the 2′-position, e.g., 2′-amino (2′-NH₂) and 2′-fluoro (2′-F), backbone modifications, methylations, unusual base-pairing combinations and the like. For example, photocrosslinking nucleic acid ligands produced by the photoSELEX process can include a photoreactive group selected from the following: 5-bromouracil (BrU), 5-iodouracil (IT), 5-bromovinyluracil, 5-iodovinyluracil, 5-azidouracil, 4-thiouracil, 5-bromocytosine, 5-iodocytosine, 5-bromovinylcytosine, 5-iodovinylcytosine, 5-azidocytosine, 8-azidoadenine, 8-bromoadenine, 8-iodoadenine, 8-azidoguanine, 8-bromoguanine, 8-iodoguanine, 8-azidohypoxanthine, 8-bromohypoxanthine, 8-iodohypoxanthine, 8-azidoxanthine, 8-bromoxanthine, 8-iodoxanthine, 5-bromodeoxyuridine, 8-bromo-2′-deoxyadenine, 5-iodo-2′-deoxyuracil, 5-iodo-2′-deoxycytosine, 5-[(4-azidophenacyl)thio]cytosine, 5-[(4-azidophenacyl)thio]uracil, 7-deaza-7-iodoadenine, 7-deaza-7-iodoguanine, 7-deaza-7-bromoadenine, and 7-deaza-7-bromoguanine. Preferentially, the photoreactive group will absorb light in a spectrum of the wavelength that is not absorbed by the target or the non-modified portions of the oligonucleotide. In preferred embodiments of the photoSELEX process, the photoreactive nucleotides incorporated into the photocrosslinking nucleic acid ligands are 5-bromo-2′-deoxyuridine (5-BrdU) and 5-iodo-2′-deoxyuridine (5-IdU). These nucleotides can be incorporated into DNA in place of thymidine nucleotides.

Photocrosslinking nucleic acid ligands produced by the photoSELEX process have particular utility in diagnostic or prognostic medical assays. In one such embodiment, photocrosslinking nucleic acid ligands of targets implicated in disease are attached to a planar solid support in an array format, and the solid support is then contacted with a biological fluid suspected of containing the targets. The photocrosslinking nucleic acid ligands are photoactivated and the solid support is washed under very stringent, aggressive conditions (preferably under conditions that denature nucleic acids and/or proteins) in order to remove all non-specifically bound molecules; bound target is not removed because it is covalently crosslinked to nucleic acid ligand via the photoreactive group. For protein targets, target quantitation can then be achieved by using a reagent that labels all proteins with a detectable group, such as a fluorescent group. The ability to photocrosslink, followed by stringent washing, allows diagnostic and prognostic assays of unparalleled sensitivity and specificity to be performed. Arrays (also commonly referred to as “biochips” or “microarrays”) of nucleic acid ligands, including photocrosslinking nucleic acid ligands and aptamers, and methods for their manufacture and use, are described in U.S. Pat. No. 6,242,246, U.S. patent application Ser. No. 08/211,680, filed Dec. 14, 1998, now abandoned, WO 99/31275, U.S. patent application Ser. No. 09/581,465, filed Jun. 12, 2000, U.S. patent application Ser. No. 09/723,394, filed Nov. 28, 2000, and U.S. patent application Ser. No. 09/723,517, filed Nov. 28, 2000, each of which is entitled “Nucleic Acid Ligand Diagnostic Biochip.” These patent applications are referred to collectively as “the biochip applications.”

Each of the above described patent applications, many of which describe modifications of the basic SELEX procedure, are specifically incorporated by reference herein in their entirety.

Given the unique ability of the SELEX process to provide ligands for virtually any target molecule, it would be highly desirable to have an automated, high-throughput method for generating nucleic acid ligands, including photocrosslinking nucleic acid ligands.

### SUMMARY OF THE INVENTION

The present invention includes methods and apparatus for the automated generation of nucleic acid ligands against virtually any target molecule. This process is termed the automated SELEX process. In its most basic embodiment, the method uses one or more robotic manipulators to move reagents to one or more work stations on a work surface where the individual steps of the SELEX process are performed.

In one series of embodiments, non-photocrosslinking aptamers of targets are generated using the automated SELEX process. The process of automatically generating non-photocrosslinking nucleic acid ligands is referred to as the automated affinity SELEX process. In one embodiment of the automated affinity SELEX process, the individual steps include: 1) contacting a candidate mixture of nucleic acids with a target molecule(s) of interest immobilized on a solid support(s) wherein nucleic acid-target complexes form; 2) partitioning the solid support(s) from the candidate mixture whereby nucleic acid-target complexes are partitioned from the remainder of the candidate mixture; and 3) amplifying the nucleic acids in the partitioned nucleic acid-target complexes. Steps 1-3 are performed for the desired number of cycles by the automated apparatus; the resulting nucleic acid ligands are then isolated and purified.

In another series of embodiments, photocrosslinking nucleic acid ligands of targets are generated using the automated photoSELEX process. In one embodiment of the automated photoSELEX process, the individual steps include: 1) contacting a candidate mixture of nucleic acids comprising one or more modified nucleotides with photoreactive groups with a target molecule(s) of interest immobilized on a solid support(s) wherein nucleic acid-target complexes form; 2) irradiating the nucleic acid-target complexes wherein the nucleic acid-target complexes photocrosslink; 3) partitioning the solid supports from the candidate mixture whereby immobilized photocrosslinked nucleic acid-target complexes are partitioned from the remainder of the candidate mixture; and 4) amplifying the nucleic acids in the partitioned nucleic acid-target complexes. Steps 1-4 are performed for the desired number of cycles by the automated apparatus; the resulting photocrosslinking nucleic acid ligands are then isolated and purified. This embodiment is referred to as the automated immobilized photoSELEX process. In preferred embodiments of the automated immobilized photoSELEX process, the candidate mixture is DNA comprising the modified nucleotide 5-bromo-2′deoxyuridine as the photoreactive group.

In another embodiment of the automated photoSELEX process, the individual steps include: 1) contacting a candidate mixture of nucleic acids comprising one or more modified nucleotides with photoreactive groups with the target molecule in solution, wherein nucleic acids having an increased affinity to said target relative to the candidate mixture form nucleic acid-target complexes; 2) irradiating the nucleic acid-target complexes, wherein the nucleic acid-target complexes photocrosslink; 3) immobilizing the photocrosslinked nucleic acid-target complexes on a solid support; 4) partitioning the solid supports from the candidate mixture whereby immobilized photocrosslinked nucleic acid-target complexes are partitioned from the remainder of the candidate mixture; and 5) amplifying the nucleic acids in the partitioned nucleic acid-target complexes. Steps 1-5 are performed for the desired number of cycles by the automated SELEX process and apparatus; the resulting photocrosslinking nucleic acid ligands are then isolated and purified. This embodiment is referred to as the automated solution photoSELEX process. In preferred embodiments of the automated solution photoSELEX process, the candidate mixture is DNA comprising the modified nucleotide 5-bromo-2′deoxyuridine.

In preferred embodiments, the automated or manual affinity SELEX process is used to produce a ligand-enriched mixture of nucleic acids that is then used as the initial candidate mixture for the automated solution photoSELEX process or the automated immobilized photoSELEX process.

The automated SELEX process described herein enables the generation of large pools of nucleic acid ligands with little or no operator intervention. In particular, the methods provided by this invention allow high affinity nucleic acid ligands to be generated routinely in just hours or a few days, rather than over a period of weeks or even months as was previously required. The highly parallel nature of the automated SELEX process allows the simultaneous isolation of ligands against diverse targets in a single automated SELEX process experiment. Similarly, the automated SELEX process can be used to generate nucleic acid ligands against a single target using many different selection conditions in a single experiment. The present invention includes examples of such highly parallel automated SELEX processes in which photocrosslinking nucleic acid ligands (photoaptamers) of multiple different targets were obtained in a single experiment using the automated solution photoSELEX process in a 96-well format. Also included are the sequences of photocrosslinking nucleic acid ligands generated according to the methods described herein to the following proteins: human neutrophil elastase (hNE), HIV-1_{MN} gp120, human L-Selectin, human P-Selectin, human platelet-derived growth factor (PDGF), human alpha-thrombin, human basic fibroblast growth factor (bFGF), HIV-1_{MN} gpl120, Angiogenin, Interleukin-4, β-Nerve Growth Factor (β-NGF), Tansforming Growth Factor β1 (TGF-β1), Interleukin-7, Kininogen, Plasmin, Serum Amyloid P, Thrombopoietin (Tpo), Coagulation Factor IX, Coagulation Factor XII, Endostatin, Factor H, Collagen, Cytotoxic T lymphocyte-associated protein-4 Fc (CTLA-4 Fc), Hepatocyte Growth Factor (HGF), Insulin-like growth factor binding protein-3 (IGFBP-3), UDP-glucuronosyl transferase (UGT) 1A1, UGT 1A10, and UGT 1A3.

The present invention greatly enhances the power of the SELEX process, and will make the automated SELEX process the routine method for the isolation of ligands.

### DETAILED DESCRIPTION OF THE FIGURES

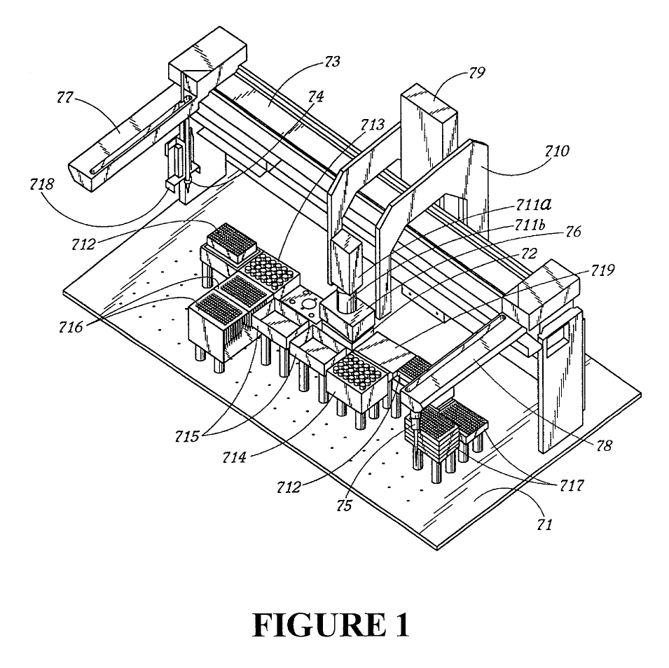

FIG. 1 shows a perspective view of an embodiment of an apparatus for performing the automated affinity SELEX process according to the present invention.

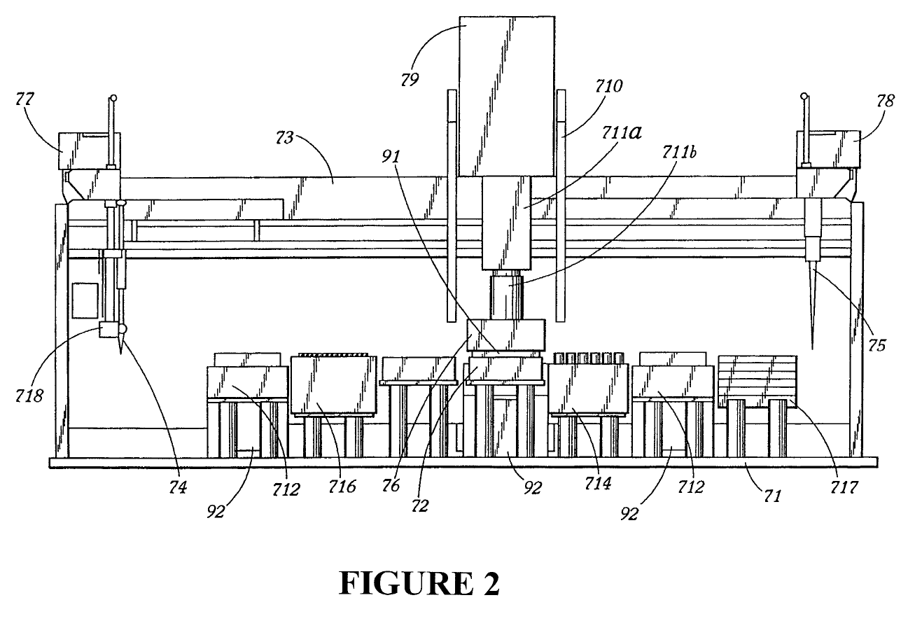

FIG. 2 shows a front elevation view the apparatus shown in FIG. 1.

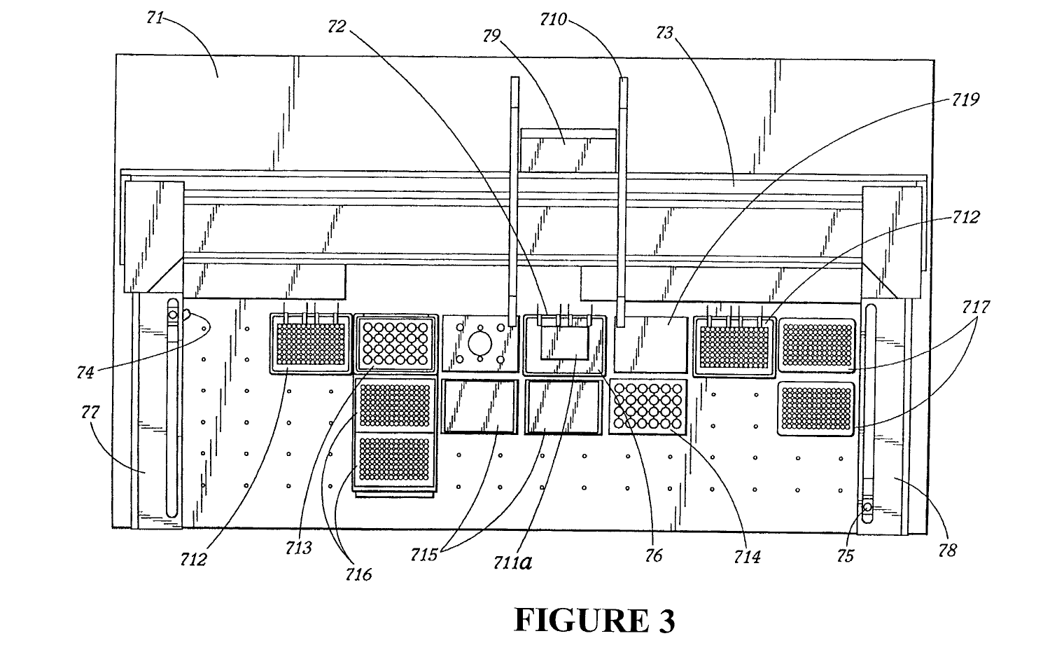

FIG. 3 shows a plan elevation view of the apparatus shown in FIG. 1.

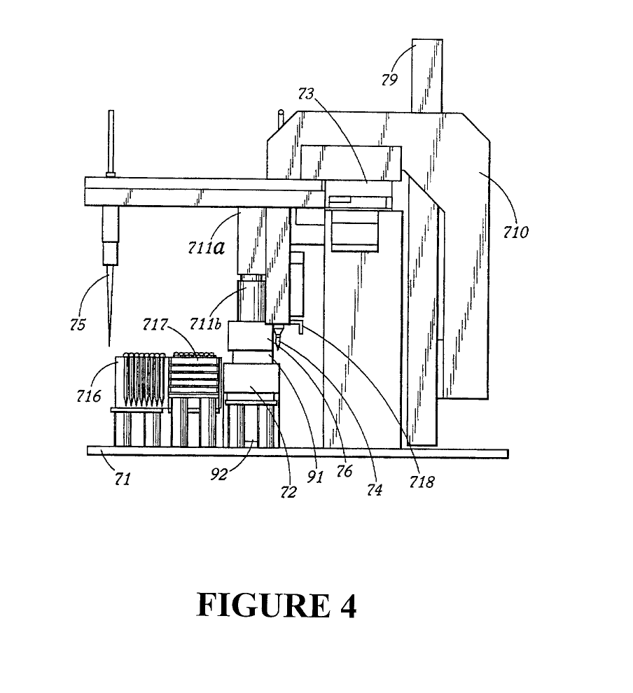

FIG. 4 shows a right side elevation view of the apparatus shown in FIG. 1.

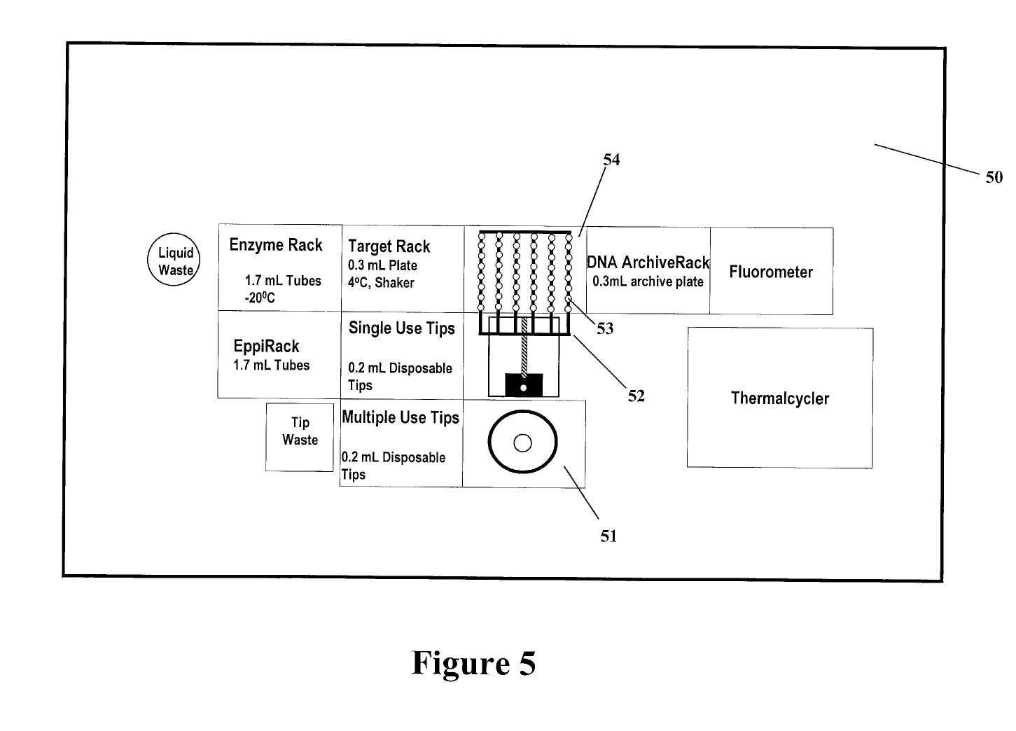

FIG. 5 shows an embodiment of an automated affinity SELEX process work surface in plan view.

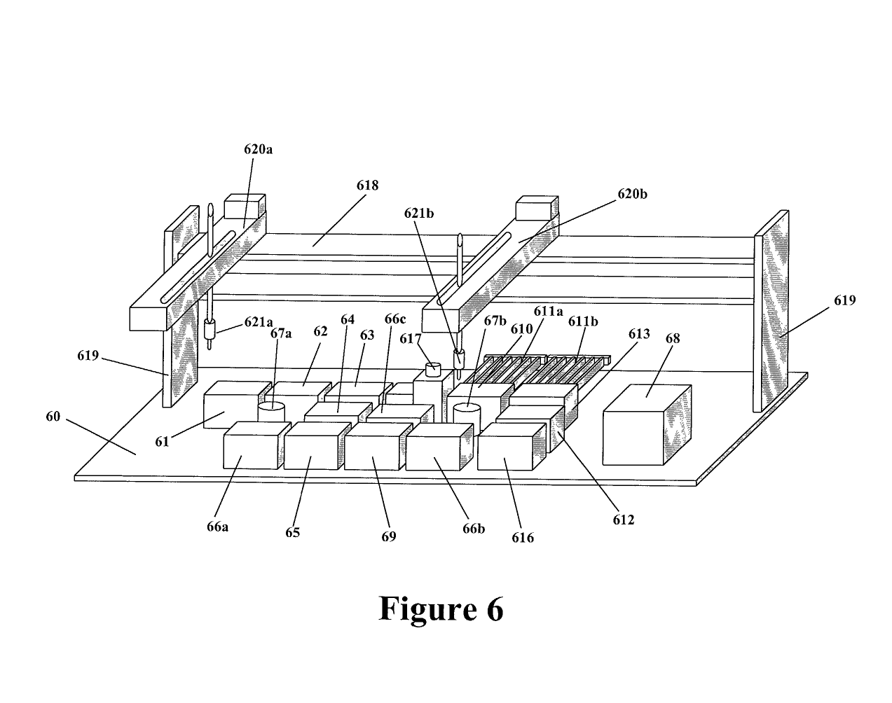

FIG. 6 shows schematically in perspective view an embodiment of an apparatus for performing the automated affinity SELEX process, the automated immobilized photoSELEX process, and the automated solution photoSELEX process.

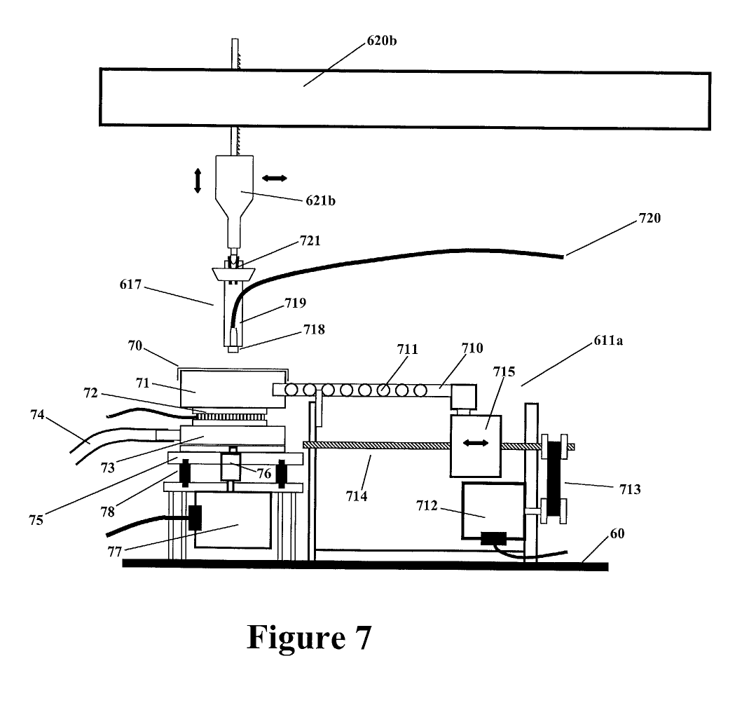

FIG. 7 illustrates a right side elevation view of the selectionModule of FIG. 6, including the magnet slider.

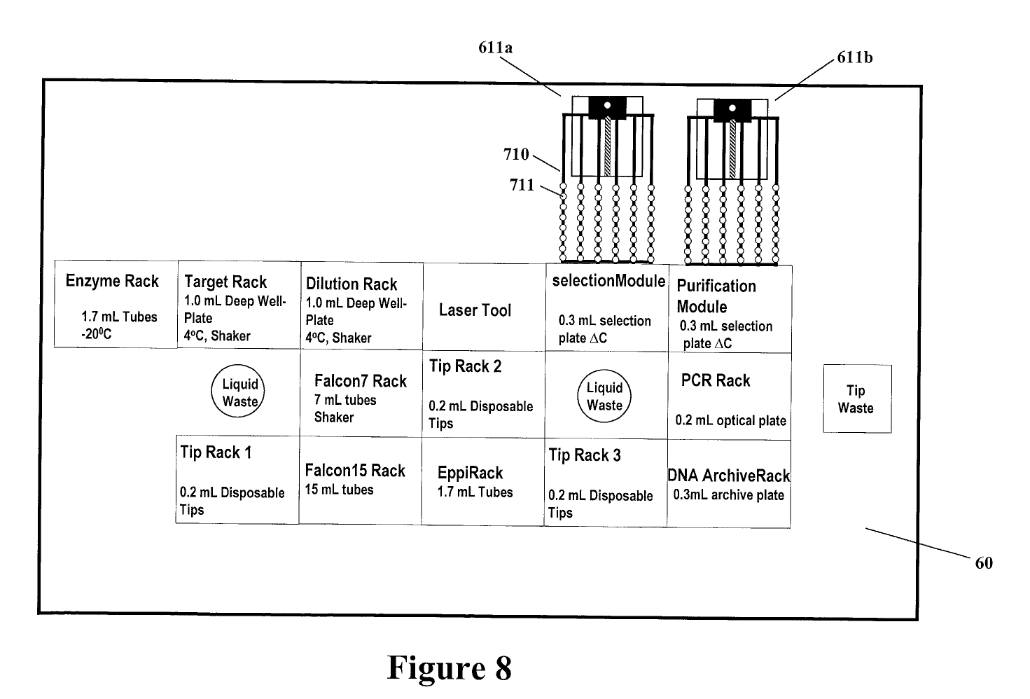

FIG. 8 shows schematically a plan elevation view of the apparatus shown in FIG. 6.

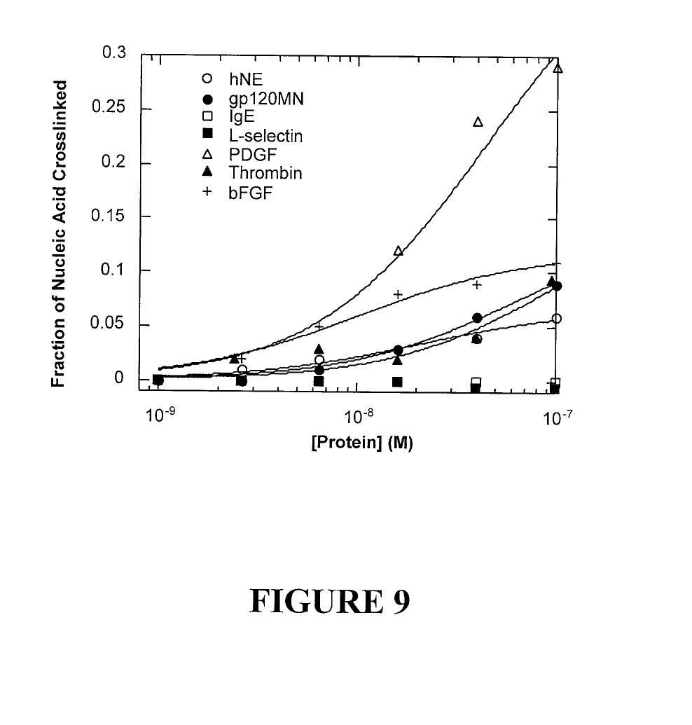

FIG. 9 shows a plot of protein concentration (M) against fraction of nucleic acid that has photocrosslinked to protein. The plot shows data for photocrosslinking nucleic acid ligands to human neutrophil elastase (hNE), HIV-1_{MN} gp120, IgE, L-Selectin, Platelet-Derived Growth Factor (PDGF), thrombin, and basic Fibroblast Growth Factor (bFGF).

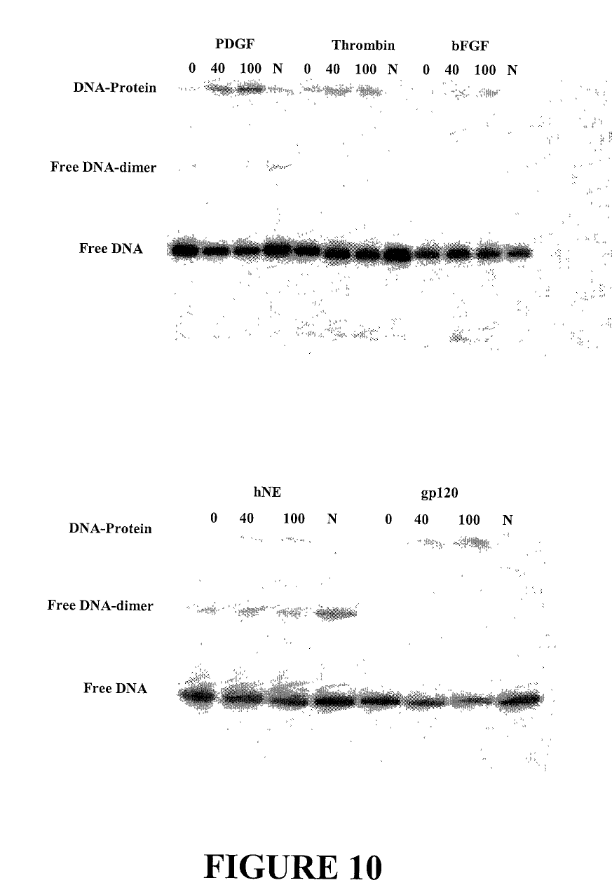

FIG. 10 shows crosslinked data on a gel for photocrosslinking nucleic acid ligands generated using the solution photoSELEX process to PDGF, Thrombin, bFGF, hNE, and gp120_{MN}. Each protein is present at 0, 40, and 100 nM; in addition, a no irradiation (N) control is also shown.

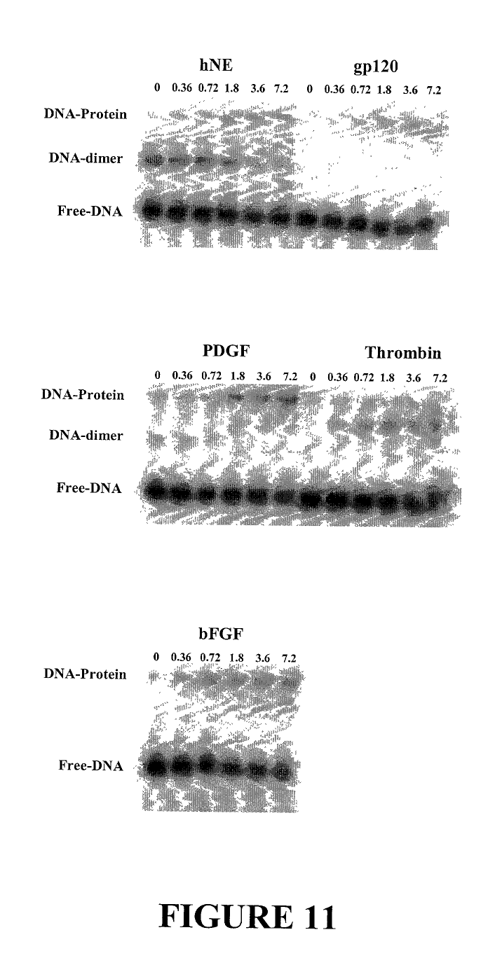

FIG. 11 shows crosslinked data on a gel for photocrosslinking nucleic acid ligands generated using the solution photoSELEX process to PDGF, Thrombin, bFGF, hNE, and gp120_{MN}. Each protein is present at 100 nM; the extent of irradiation is varied for each protein.

### DETAILED DESCRIPTION OF THE INVENTION

Definitions

Various terms are used herein to refer to aspects of the present invention. To aid in the clarification of the description of the components of this invention, the following definitions are provided:

As used herein, “nucleic acid ligand” is a non-naturally occurring nucleic acid having a desirable action on a target. Nucleic acid ligands are also sometimes referred to in this application as “aptamers” or “clones.” A desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. In the preferred embodiment, the action is specific binding affinity for a target molecule, such target molecule being a three dimensional chemical structure other than a polynucleotide that binds to the nucleic acid ligand through a mechanism which predominantly depends on Watson/Crick base pairing or triple helix binding, wherein the nucleic acid ligand is not a nucleic acid having the known physiological function of being bound by the target molecule. Nucleic acid ligands include nucleic acids that are identified from a candidate mixture of nucleic acids, said nucleic acid ligand being a ligand of a given target, by the method comprising: a) contacting the candidate mixture with the target, wherein nucleic acids having an increased affinity to the target relative to the candidate mixture may be partitioned from the remainder of the candidate mixture; b) partitioning the increased affinity nucleic acids from the remainder of the candidate mixture; and c) amplifying the increased affinity nucleic acids to yield a ligand-enriched mixture of nucleic acids, whereby nucleic acid ligands of the target molecule are identified.

As used herein, “candidate mixture” is a mixture of nucleic acids of differing sequence from which to select a desired ligand. The source of a candidate mixture can be from naturally-occurring nucleic acids or fragments thereof, chemically synthesized nucleic acids, enzymatically synthesized nucleic acids or nucleic acids made by a combination of the foregoing techniques. Modified nucleotides, such as nucleotides with photoreactive groups, can be incorporated into the candidate mixture. In addition, a candidate mixture can be produced by a prior SELEX process e.g., a first SELEX process experiment can be used to produce a ligand-enriched mixture of nucleic acids that is then used as the candidate mixture in a second SELEX process experiment. A candidate mixture can also comprise nucleic acids with one or more common structural motifs. For example, U.S. Provisional Patent Application Serial No. 60/311,281, filed Aug. 9, 2001, entitled “Nucleic Acid Ligands With Intramolecular Duplexes” and incorporated herein by reference in its entirety, describes candidate mixtures comprising nucleic acids with intramolecular duplexes formed between their 5′ and 3′ ends.

In this invention, candidate mixture is also sometimes referred to as “pool” or “library.” For example, “RNA pool” refers to a candidate mixture comprised of RNA.

In a preferred embodiment, each nucleic acid has fixed sequences surrounding a randomized region to facilitate the amplification process. As detailed elsewhere in this application, the candidate mixture nucleic acids can further comprise fixed “tail” sequences at their 5′ and 3′ termini to prevent the formation of high molecular weight parasites of the amplification process.

As used herein, “nucleic acid” means either DNA, RNA, single-stranded or double-stranded, and any chemical modifications thereof. Modifications include, but are not limited to, those which provide other chemical groups that incorporate additional charge, polarizability, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

“SELEX” methodology involves the combination of selection of nucleic acid ligands which interact with a target in a desirable manner, for example binding to a protein, with amplification of those selected nucleic acids. Optional iterative cycling of the selection/amplification steps allows selection of one or a small number of nucleic acids which interact most strongly with the target from a pool which contains a very large number of nucleic acids. Cycling of the selection/amplification procedure is continued until a selected goal is achieved. The SELEX methodology is described in the SELEX Patent Applications. In some embodiments of the SELEX process, aptamers that bind non-covalently to their targets are generated. In other embodiments of the SELEX process, aptamers that bind covalently to their targets are generated.

“SELEX target” or “target molecule” or “target” refers herein to any compound upon which a nucleic acid can act in a predetermined desirable manner. A SELEX target molecule can be a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, etc., without limitation. Virtually any chemical or biological effector would be a suitable SELEX target. Molecules of any size can serve as SELEX targets. A target can also be modified in certain ways to enhance the likelihood of an interaction between the target and the nucleic acid. Embodiments of the SELEX process in which the target is a peptide are described in U.S. patent application Ser. No. 09/668,602, filed Sep. 22, 2000, entitled “Modified SELEX Processes Without Purified Protein,” incorporated herein by reference in its entirety.

“Tissue target” or “tissue” refers herein to a certain subset of the SELEX targets described above. According to this definition, tissues are macromolecules in a heterogeneous environment. As used herein, tissue refers to a single cell type, a collection of cell types, an aggregate of cells, or an aggregate of macromolecules. This differs from simpler SELEX targets which are typically isolated soluble molecules, such as proteins. In the preferred embodiment, tissues are insoluble macromolecules which are orders of magnitude larger than simpler SELEX targets. Tissues are complex targets made up of numerous macromolecules, each macromolecule having numerous potential epitopes. The different macromolecules which comprise the numerous epitopes can be proteins, lipids, carbohydrates, etc., or combinations thereof. Tissues are generally a physical array of macromolecules that can be either fluid or rigid, both in terms of structure and composition. Extracellular matrix is an example of a more rigid tissue, both structurally and compositionally, while a membrane bilayer is more fluid in structure and composition. Tissues are generally not soluble and remain in solid phase, and thus partitioning can be accomplished relatively easily. Tissue includes, but is not limited to, an aggregate of cells usually of a particular kind together with their intercellular substance that form one of the structural materials commonly used to denote the general cellular fabric of a given organ, e.g., kidney tissue, brain tissue. The four general classes of tissues are epithelial tissue, connective tissue, nerve tissue and muscle tissue.

Examples of tissues which fall within this definition include, but are not limited to, heterogeneous aggregates of macromolecule such as fibrin clots which are acellular; homogeneous or heterogeneous aggregates of cells; higher ordered structures containing cells which have a specific function, such as organs, tumors, lymph nodes, arteries, etc.; and individual cells. Tissues or cells can be in their natural environment, isolated, or in tissue culture. The tissue can be intact or modified. The modification can include numerous changes such as transformation, transfection, activation, and substructure isolation, e.g., cell membranes, cell nuclei, cell organelles, etc.

Sources of the tissue, cell or subcellular structures can be obtained from prokaryotes as well as eukaryotes. This includes human, animal, plant, bacterial, fungal and viral structures.

As used herein, “solid support” is defined as any surface to which molecules may be attached through either covalent or non-covalent bonds. This includes, but is not limited to, membranes, plastics, paramagnetic beads, charged paper, nylon, Langmuir-Bodgett films, functionalized glass, germanium, silicon, PTFE, polystyrene, gallium arsenide, gold and silver. Any other material known in the art that is capable of having functional groups such as amino, carboxyl, thiol or hydroxyl incorporated on its surface, is also contemplated. This includes surfaces with any topology, including, but not limited to, spherical surfaces, grooved surfaces, and cylindrical surfaces e.g., columns.

“Partitioning” means any process whereby ligands bound to target molecules can be separated from nucleic acids not bound to target molecules. More broadly stated, partitioning allows for the separation of all the nucleic acids in a candidate mixture into at least two pools based on their relative affinity to the target molecule. Partitioning can be accomplished by various methods known in the art. Nucleic acid-protein pairs can be bound to nitrocellulose filters while unbound nucleic acids are not. Columns which specifically retain nucleic acid-target complexes can be used for partitioning. For example, oligonucleotides able to associate with a target molecule bound on a column allow use of column chromatography for separating and isolating the highest affinity nucleic acid ligands. Beads upon which target molecules are conjugated can also be used to partition nucleic acid ligands in a mixture. If the beads are paramagnetic, then the partitioning can be achieved through application of a magnetic field. Surface plasmon resonance technology can be used to partition nucleic acids in a mixture by immobilizing a target on a sensor chip and flowing the mixture over the chip, wherein those nucleic acids having affinity for the target can be bound to the target, and the remaining nucleic acids can be washed away. Liquid-liquid partitioning can be used as well as filtration gel retardation, and density gradient centrifugation.

As used herein, “PhotoSELEX” is an acronym for Photochemical Systematic Evolution of Ligands by EXponential enrichment, and refers to embodiments of the SELEX process in which photocrosslinking aptamers are generated. In the photoSELEX process, a photoreactive nucleotide activated by absorption of light is incorporated in place of a native base in either RNA- or in ssDNA-randomized oligonucleotide libraries, the nucleic acid target molecule mixture is irradiated causing some nucleic acids incorporated in nucleic acid-target molecule complexes to crosslink to the target molecule via the photoreactive functional groups, and the selection step is a selection for photocrosslinking activity. The photoSELEX process is described in great detail in the PhotoSELEX Process Applications.

In this application, the term “the affinity SELEX process” refers to embodiments of the SELEX process in which non-photocrosslinking aptamers of targets are generated. In preferred embodiments of the affinity SELEX process, the target is immobilized on a solid support either before or after the target is contacted with the candidate mixture of nucleic acids. The association of the target with the solid support allows nucleic acids in the candidate mixture that have bound to target to be partitioned from the remainder of the candidate mixture. The term “bead affinity SELEX process” refers to particular embodiments of the affinity SELEX process where the target is immobilized on a bead, preferably before contact with the candidate mixture of nucleic acids. Preferred beads include paramagnetic beads. The term “filter affinity SELEX process” refers to embodiments where nucleic acid target complexes are partitioned from candidate mixture by virtue of their association with a filter, such as a nitrocellulose filter. This includes embodiments where target and nucleic acids are initially contacted in solution, then contacted with the filter, and also embodiments where nucleic acids are contacted with target that is pre-immobilized on the filter. The term “plate affinity SELEX process” refers to embodiments where target is immobilized on the surface of a plate, preferably a multi-well microtiter plate. Preferably, the plate is comprised of polystyrene. Target is preferably attached to the plate in the plate affinity SELEX process through hydrophobic interactions.

The SELEX Patent Applications and the PhotoSELEX Process Applications describe and elaborate on the aforementioned processes in great detail. Included are targets that can be used; methods for the preparation of the initial candidate mixture; methods for partitioning nucleic acids within a candidate mixture; and methods for amplifying partitioned nucleic acids to generate enriched candidate mixtures. The SELEX Patent Applications and the PhotoSELEX Process Applications also describe ligand solutions obtained to a number of target species, including protein targets wherein the protein is or is not a nucleic acid binding protein.

Note that throughout this application, various publications, publications, and patent applications are mentioned; each is incorporated by reference to the same extent as if each was specifically and individually incorporated by reference.

A. The Basic Automated SELEXProcess

In its most basic form, the SELEX process may be defined by the following series of steps:

1) A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: a) to assist in the amplification steps described below; b) to mimic a sequence known to bind to the target; or c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent. Additional fixed “tail” sequences may be added to the 5′ and 3′ termini of the candidate mixture nucleic acids to prevent high molecular weight artifacts of the amplification process from forming when the amplification process is not followed by size fractionation of the amplified mixture. Such tail sequences, and other methods for preventing high molecular weight artifacts (termed “parasites”), are described in U.S. patent application Ser. No. 09/616,284, filed Jul. 14, 2000, and in U.S. patent application Ser. No. 09/815,171, filed Mar. 22, 2001, each of which is entitled “Method and Apparatus for the Automated Generation of Nucleic Acid Ligands.”

2) The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.

3) The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a certain amount of the nucleic acids in the candidate mixture are retained during partitioning.

4) Those nucleic acids selected during partitioning as having relatively higher affinity to the target are then amplified to create a new candidate mixture that is enriched in nucleic acids having a relatively higher affinity for the target. The primers used for amplification also preferably have “tail” sequences at their 5′ ends in order to prevent the formation of high molecular weight parasites of the amplification process. Such primers are also described in U.S. patent application Ser. No. 09/616,284, filed Jul. 14, 2000, and in U.S. patent application Ser. No. 09/815,171, filed Mar. 22, 2001, each of which is entitled “Method and Apparatus for the Automated Generation of Nucleic Acid Ligands.”

5) By repeating the partitioning and amplifying steps above, the newly formed candidate mixture contains fewer and fewer unique sequences, and the average degree of affinity of the nucleic acids to the target will generally increase. Taken to its extreme, the SELEX process will yield a candidate mixture containing one or a small number of unique nucleic acids representing those nucleic acids from the original candidate mixture having the highest affinity to the target molecule. The aforementioned steps are central to all specific embodiments of the SELEX process, including the affinity SELEX process and the photoSELEX process.

In some embodiments of the automated SELEX process, steps 2)-5) are performed automatically by one or more computer-controlled robotic manipulators; in other embodiments, amplification step 4) is performed manually while the other steps are performed automatically by one or more computer-controlled robotic manipulators. In some embodiments, the computer also measures and stores information about the progress of the automated SELEX process, including the amount of nucleic acid ligand eluted from the target molecule prior to each amplification step. The computer also controls the various heating and cooling steps required for the automated SELEX process.

In one embodiment, the computer-controlled robotic manipulator(s) moves solutions to and from a work station (also referred to herein as a “module”) located on a work surface. In preferred embodiments, the work surface comprises a single work station or module where the individual SELEX process reactions take place. This work station or module preferably comprises heating and cooling means controlled by the computer in order to incubate the reaction mixtures at the required temperatures. One suitable heating and cooling means is a Peltier element. The work station preferably also comprises a shaking mechanism to insure that SELEX reaction components are adequately mixed. In addition, the work station preferably comprises an array of magnets on sliders for partitioning paramagnetic beads (see below in the section entitled “The Automated Affinity SELEX Process”). The work surface also comprises other stations in which the enzymes necessary for the SELEX process are stored under refrigeration, stations where wash solutions and buffers are stored, stations where tools and apparatus are stored, stations where tools and apparatus may be rinsed, and stations where pipette tips and reagents are discarded. The work surface may also comprise stations for archival storage of small aliquots of the SELEX process reaction mixtures. These mixtures may be automatically removed from the work station by the pipetting tool at selected times for later analysis. The work surface may also comprise reagent preparation and dilution stations where the robotic manipulator prepares batches of enzyme reagent solutions and buffer solutions in preparation vials immediately prior to use.

In other embodiments, the work surface comprises more than one work station or module. In this way, it is possible to perform the individual steps of the automated SELEX process asynchronously. For example, while a first set of candidate nucleic acid ligands is being amplified on a first work station, another set from a different experiment may be contacted with target molecule on a different work station. Using multiple work stations minimizes the idle time of the robotic manipulator. FIGS. 1-5 illustrate one embodiment of the work surface comprising a central module (a shaker for holding a microtiter plate, and heating/cooling means), a thermal cycler (capable of performing PCR), and reagent and tip racks.

In still other embodiments, the individual steps of the automated SELEX process are carried out at discrete work stations rather than at a single multi-functional work station. In these embodiments, the solutions of candidate nucleic acid mixtures can be transferred from one work station to another by the robotic manipulator. Separate work stations may be provided for heating and cooling the reaction mixtures. Additionally, one work station may be provided for the incubation of candidate mixtures of nucleic acid ligands with target molecules, while another work station is provided for the purification of newly-amplified nucleic acid ligands from amplification reactions; FIGS. 6-8 and Example 2 illustrate an embodiment of the invention with two such work stations referred to as “selectionModule” and “purificationModule” respectively.

In preferred embodiments, the individual steps of the automated SELEX process are carried out in a containment vessel that is arranged in an array format. This allows many different SELEX reactions—using different targets or different reaction conditions—to take place simultaneously on a single work station. For example, in some embodiments the individual steps may be performed in the wells of microtitre plates, such as Immulon 1 plates. In other embodiments, an array of small plastic tubes is used. Typical tube arrays comprise 96 0.5 ml round-bottomed, thin-walled polypropylene tubes laid out in a 8×12 format. Arrays can be covered during the heating and cooling steps to prevent liquid loss through evaporation, and also to prevent contamination. Any variety of lids, including heated lids, can be placed over the arrays by the robotic manipulator during these times. Furthermore, arrays allow the use of multipipettor devices, which can greatly reduce the number of pipetting steps required. For the purposes of this specification, the term “well” will be used to refer to an individual containment vessel in any array format.

In some embodiments, each robotic manipulator is a movable arm that is capable of carrying tools in both horizontal and vertical planes i.e. in x-y-z planes. One tool contemplated is a pipetting tool. A robotic manipulator uses the pipetting tool to pick up liquid from a defined location on the work surface and then dispense the liquid at a different location. The pipetting tool can also be used to mix liquids by repeatedly picking up and ejecting the liquid i.e. “sip and spit” mixing. The robotic manipulator is also able to eject a disposable tip from the pipetting tool into a waste container, and then pick up a fresh tip from the appropriate station on the work surface.

In preferred embodiments, the pipetting tool is connected to one or more fluid reservoirs that contain some of the various buffers and reagents needed in bulk for the SELEX process. A computer controlled valve determines which solution is dispensed by the pipetting tool. The pipetting tool is further able to eject liquid at desired locations on the work surface without the outside of the tip coming in contact with liquid already present at that location. This greatly reduces the possibility of the pipette tip becoming contaminated at each liquid dispensing step, and reduces the number of pipette tip changes that must be made during the automated SELEX process.

In some embodiments, tips that are used at certain steps of the automated SELEX process can be reused. For example, a tip can be reused if it is used in each cycle of the SELEX process to dispense the same reagent. The tip can be rinsed after each use at a rinse station, and then stored in a rack on the work surface until it is needed again. Reusing tips in this way can drastically reduce the number of tips used during the automated SELEX process.

In preferred embodiments, a vacuum aspiration system is also attached to a separate robotic manipulator. This system uses a fine needle connected to a vacuum source to withdraw liquid from desired locations on the work surface without immersing the needle in that liquid. In embodiments where the pipetting tool and the vacuum aspirator are associated with separate robotic manipulators, the pipetting tool and the aspiration system can work simultaneously at different locations on the work surface. In other embodiments, a vacuum aspirating tool comprising a fine needle connected to a vacuum source can be picked up by a pipetting tool. The vacuum aspiration tool can comprise an embedded pipette tip to allow the pipetting tool to pick it up. In other embodiments, the pipetting tool itself aspirates liquid, which liquid is then dispensed into a waste liquid container.

In preferred embodiments, a robotic manipulator is also capable of moving objects to and from defined locations on the work surface. Such objects include lids for multi-well plates, and also the various pieces of apparatus used in the embodiments outlined below, e.g., the laser tool in the automated photoSELEX process as outlined below. In one embodiment of the invention, the robotic manipulator uses a “gripper” to mechanically grasp such object. Such a gripper is shown in FIG. 1. In other embodiments, the vacuum aspiration system described above is also used to power a suction cup that can attach to the object to be moved. For example, the fine needle described above can pick up a suction cup, apply a vacuum to the cup, pick up an object using the suction cup, move the object to a new location, release the object at the new location by releasing the vacuum, then deposit the suction cup at a storage location on the work surface.

In some embodiments, the amplification of candidate nucleic acid ligands that takes place at step 4) above is performed on a commercially-available thermal cycler located off or on the worksurface. In embodiments in which candidate nucleic acid ligands are held in multi-well plates, the entire plate can be transferred to the thermal cycler either by the robot, or manually by the operator.

In other embodiments, the robotic manipulator(s) perform only liquid manipulations (including pipetting, aspiration, and “sip and spit” mixing), and irradiation of the individual wells of microtiter plates (in the automated photoSELEX process described below). Such manipulations are by far the most time consuming if performed manually. Other manipulations can be performed manually without any loss in the throughput efficiency of the automated SELEX process. For example, movement of multi-well plates to heating and cooling blocks, or to thermal cyclers, can be performed manually. Such heating and cooling blocks, and thermal cyclers, can be located off the work surface. The robot layout in FIGS. 6-8 illustrates one such embodiment in which thermal cycling of PCR reaction is performed off the work surface by manually transferring multi-well plates.

Suitable robotic systems contemplated in the invention include, but are not limited to, the MultiPROBE™ system (Packard), the Biomek 200™ (Beckman Instruments) and the Tecan™ (Cavro). Non-limiting, exemplary robot layouts are depicted in FIGS. 1-8.

Having described basic design considerations of the apparatus for carrying out the automated SELEX process, the following sections discuss more specifically apparatus design and methods for the automated generation of aptamers according to particular embodiments of the automated SELEX process: the automated affinity SELEX, the automated immobilized photoSELEX process, and the automated solution photoSELEX process.

B. The Automated Affinity SELEXProcess

The following is a more detailed description of apparatus design and methods for particular embodiments of the automated SELEX process in which non-photocrosslinking aptamers are produced. Such embodiments are referred to as the automated affinity SELEX processes. It is to be understood that many elements of the apparatus and many of the individual steps of the methods are equally applicable to the automated photoSELEX process. The automated photoSELEX process is described in great detail later in this application.

One embodiment of the automated affinity SELEX process includes the steps of:

(a) contacting a candidate mixture of nucleic acid ligands in a containment vessel with a target molecule that is associated with a solid support;

(b) incubating the candidate mixture and the solid support in the containment vessel at a predetermined temperature to allow candidate nucleic acid ligands to interact with the target;

(c) partitioning the solid support with bound target and associated nucleic acid ligands from the candidate mixture;

(d) optionally washing the solid support under predetermined conditions to remove nucleic acids that are associated non-specifically with the solid support or the containment vessel;

(e) releasing from the solid support the nucleic acid ligands that interact specifically with the target;

(f) amplifying, purifying and quantifying the released nucleic acid ligands;

(g) repeating steps (a)-(f) a predetermined number of times; and

(h) isolating the resulting nucleic acid ligands.

Solid supports suitable for attaching target molecules are well known in the art. Any solid support to which a target molecule can be attached, either covalently or non-covalently, is contemplated by the present invention. Covalent attachment of molecules to solid supports is well known in the art, and can be achieved using a wide variety of derivatization chemistries. Non-covalent attachment of targets can depend on hydrophobic interactions; alternatively, the solid support can be coated with streptavidin which will bind strongly to a target molecule that is conjugated to biotin. Non-limiting, exemplary methods for biotinylation of target proteins are provided in the Examples section of this application.

In preferred embodiments, protein target molecules are covalently attached to a solid support using a benzophenone-based crosslinker. For example, the succinimidyl ester of 4-benzoylbenzoic acid can be coupled to paramagnetic beads functionalized with primary amino groups. When the resulting beads are mixed with target protein and irradiated with 360 nm light, the benzophenone is photoactivated and covalently attaches to the protein. Methods for the synthesis of benzophenone-based crosslinkers are provided in U.S. patent application Ser. No. 09/815,171, filed Mar. 22, 2001, entitled “Method and Apparatus for the Automated Generation of Nucleic Acid Ligands.”

The conformation adopted by the target on a solid support may vary slightly depending on the nature of coupling chemistry. In some very rare instances, it might be expected that the some coupling chemistries will produce immobilized target that has a sufficiently different conformation from native protein that the resulting nucleic acid ligands bind poorly to the native target. In order to avoid this outcome, in some embodiments of the affinity SELEX process target molecules are coupled to solid supports using more than one coupling chemistry. Using multiple coupling chemistries in a single SELEX process experiment increases the probability of obtaining a nucleic acid ligand that can bind to the native target by increasing the chance that one of the coupling chemistries will present the target in the same conformation as the native target. Example 15 below illustrates one such embodiment in which the bead affinity SELEX process was performed using streptavidin beads and target protein biotinylated in three different ways. Target protein was biotinylated according to example 6 above either through carboxyl groups, carbohydrate groups, or by using a photobiotinylation protocol.

In preferred embodiments, the solid support is a bead. We refer to such embodiments of the automated affinity SELEX process that employ beads as “the automated bead affinity SELEX process.” In some embodiments, the bead is non-paramagnetic, and solutions are removed from the wells by aspirating the liquid through a hole in the well that is small enough to exclude the passage of the beads. For example, a vacuum manifold with a 0.2 μm filter could be used to partition 100 μm beads. Most preferably, the beads are paramagnetic beads, such as those available from Dynal, Inc. When target molecules are attached to paramagnetic beads, complexes of target molecules and nucleic acid ligands can be rapidly partitioned from the candidate mixture by the application of a magnetic field to the wells. In preferred embodiments, the magnetic field is applied by an array of electromagnets adjacent to the walls of each well; when the electromagnets are activated by the computer, paramagnetic target beads are held to the sides of the wells. The magnets can either be an integral part of the work station(s), or they can be attached to a cover that is lowered over the work station by the robotic manipulator. In this latter embodiment, the magnetic separator cover allows the magnets to be placed adjacent to the wells without blocking access to the wells themselves. In this way, the wells are accessible by the pipetting and aspirating units when the cover is in place. Following magnet activation, liquid can be aspirated from the wells, followed by the addition of wash solutions. When the electromagnets are deactivated, or when the cover is removed, the beads become resuspended in the solution. The magnetic separator cover can be stored on the work surface. In other embodiments, the magnets in the separator cover are permanent magnets. In this case, withdrawing the cover removes the influence of the magnets, and allows the beads to go into suspension.

In especially preferred embodiments, permanent magnets are attached to a series of bars that can slide between adjacent rows of wells. Each bar has magnets regularly spaced along its length, such that when the bar is fully inserted between the wells, each well is adjacent to at least one magnet. For example, an 8×12 array of wells could have 8 magnet bars, each bar with 12 magnets. Alternatively, an 8×12 array of wells could have 6 magnet bars, each bar with 8 magnets as shown in FIGS. 6-8. In embodiments using magnet bars, bead separation is achieved by inserting the bars between the wells; bead release is accomplished by withdrawing the bars from between the wells. The array of bars can be moved by a computer-controlled stepper motor.

The paramagnetic target beads used in the above embodiments are preferably stored on the work surface in an array format that mirrors the layout of the array format on the work station. The bead storage array is preferably cooled, and agitated to insure that the beads remain in suspension before use.

Beads can be completely removed from the wells of the work station using a second array of magnets. In preferred embodiments, this second array comprises an array of electromagnets mounted on a cover that can be placed by the robotic manipulator over the surface of the individual wells on the work station. The electromagnets on this bead removal cover are shaped so that they project into the liquid in the wells. When the electromagnets are activated, the beads are attracted to them. By then withdrawing the bead removal cover away from the wells, the beads can be efficiently removed from the work station. The beads can either be discarded, or can be deposited back in the bead storage array for use in the next round of the automated SELEX process. The bead removal cover can then be washed at a wash station on the work surface prior to the next bead removal step.

In a typical embodiment involving paramagnetic beads, the automated affinity SELEX process begins when the pipetting tool dispenses aliquots of the beads—with their bound target—to the individual wells of a microtitre plate located on a work station or module. Each well preferably already contains an aliquot of a candidate mixture of nucleic acid ligands previously dispensed by the robotic manipulator. After dispensing the beads, the robot optionally shakes the wells to facilitate thorough mixing. The microtitre plate is then incubated at a preselected temperature on the work station in order to allow nucleic acid ligands in the candidate mixture to bind to the bead-bound target molecule. In some embodiments, the preferred temperature is room temperature; in such embodiments, it is not necessary for the work station or module where the beads and candidate mixture are contacted with one another to be associated with heating or cooling means. Agitation of the plate insures that the beads remain in suspension.

After incubation for a suitable time, a magnet bar is inserted between the wells by a computer-controlled stepper motor. The beads are then held to the sides of the wells, and the aspirator tool removes the solution containing unbound candidate nucleic acids from the wells. A washing solution, such as a low salt solution, can then be dispensed into each well by the pipetting tool. The beads are released from the side of the wells by withdrawing the magnet bar, then resuspended in the wash solution by agitation. The magnetic bar is inserted between the plate wells again, and the wash solution is aspirated. This wash loop can be repeated for a pre-selected number of cycles in order to remove all nucleic acids that are not bound specifically to the target. At the end of the wash loop, the beads are held by the magnets to the sides of the empty wells.

The beads can then be resuspended in a solution designed to release (elute) the nucleic acid ligands from the target molecule, such as dH₂O or a NaOH solution. The release of nucleic acid ligand from target can also be achieved by heating the beads to a high temperature, either on the work station (in embodiments where the work surface comprises heating and cooling means), or by manually transferring the plate to a heating block located off the work surface. Following release of the nucleic acid ligands into the solution phase, the beads can be pulled to the sides of the wells by magnets, and the solution phase can be transferred to a new microtiter plate for amplification, purification, and quantitation (see below).

A predetermined amount of the amplified candidate mixture can then used in the next round of the automated SELEX process. At any point during the automated affinity SELEX process, the pipetting tool can remove an aliquot of the candidate mixture and store it in an archive plate for later characterization. Furthermore, during incubation periods, the pipetting tool can prepare reaction mixtures for other steps in the automated affinity SELEX process.

As described above, the preferred embodiments of the automated affinity SELEX process method and apparatus use microtitre plates as containment vessels and magnetic beads as solid supports in order to achieve selection. However, any other method for partitioning bound nucleic acid ligands from unbound is contemplated in the invention. For example, in some embodiments, the target molecule is coupled directly to the surface of the microtitre plate. Suitable methods for coupling in this manner are well known in the art. In such embodiments, the plate is most preferably comprised of polystyrene and serves both as the solid support to which target is attached, and also as the containment vessel. Preferably, the target is attached to the surface of plate wells through hydrophobic interactions. We refer to embodiments of the SELEX process where the target is associated with a plate as the “plate affinity SELEX process.”

In other embodiments, the target molecule is coupled to affinity separation columns known in the art. The robotic device would dispense the candidate mixture into such a column, and the bound nucleic acid ligands could be eluted into the wells of a microtitre plate after suitable washing steps.

In still other embodiments, the solid support used in the automated affinity SELEX process method is a surface plasmon resonance (SPR) sensor chip. The use of SPR sensor chips in the isolation of nucleic acid ligands is described in WO 98/33941, entitled “Flow Cell SELEX,” incorporated herein by reference in its entirety. In the Flow Cell SELEX method, a target molecule is coupled to the surface of a surface plasmon resonance sensor chip. The refractive index at the junction of the surface of the chip and the surrounding medium is extremely sensitive to material bound to the surface of the chip. In one embodiment of the present invention, a candidate mixture of nucleic acid ligands is passed over the chip by the robotic device, and the kinetics of the binding interaction between the chip-bound target and nucleic acid ligands is monitored by taking readings of the resonance signal from the chip. Such readings can be made using a device such as the BIACore 2000™ (BIACore, Inc.). Bound nucleic acid ligands can then be eluted from the chip; the kinetics of dissociation can be followed by measuring the resonance signal. In this way it is possible to program the computer that controls the automated SELEX process to automatically collect nucleic acid ligands which have a very fast association rate with the target of interest and a slow off rate.

Nucleic acid ligands that are dissociated from solid support-bound target can be amplified as described below in the section entitled “Amplification of Candidate Nucleic Acid Ligands.” Following amplification, the automated affinity SELEX process cycle can begin again. At the end of the automated affinity SELEX process, the resulting pools of nucleic acid ligands (one pool for each target) can be removed for activity analysis, cloning, and sequencing.

C. The Automated PhotoSELEX Process

In some embodiments of the invention, nucleic acid ligands that undergo photochemical crosslinking to their targets are generated using the photoSELEX process. The photoSELEX process and photocrosslinkable nucleic acid ligands are described in great detail in the PhotoSELEX Process Applications. Any modified nucleotide residue that is capable of photocrosslinking (or chemically reacting) with a target molecule, such as 5-BrdU, 5-IdU or other 5-modified nucleotides, can be incorporated into the candidate mixture and may be useful in this application. In preferred embodiments, the crosslinking occurs when 5-bromo-2′-deoxyuridine (5-BrdU) residues incorporated into a nucleic acid ligand in place of T residues are irradiated with ultraviolet (UV) light. Photocrosslinkable nucleic acid ligands are useful because they enable assays in which very stringent (even denaturing) washes can be used to prevent non-specific interactions between targets and nucleic acid ligands. Non-limiting, exemplary methods for preparing 5-BrdU candidate DNA mixtures are provided in the Examples section in this application.

In the following embodiments, manipulations that are specific to the photoSELEX process are outlined in detail; manipulations that are common to both the automated affinity SELEX process and the automated photoSELEX process are carried out according to the methods provided in the preceding sections.

I. The Automated Immobilized PhotoSELEX Process

In some embodiments of the automated photoSELEX process, targets are immobilized on solid supports (according to methods presented elsewhere in this application e.g., using benzophenone crosslinkers, using multiple coupling chemistries), preferably paramagnetic beads, and photocrosslinking takes place on the solid supports. In these embodiments, DNA candidate mixtures with photoreactive nucleotides, preferably 5-BrdU residues in place of T residues, are dispensed to the individual wells of a microtiter plate located on the work station, along with target molecules conjugated to paramagnetic beads. Following incubation of the reaction mixtures, the wells of the microtiter plate are irradiated with UV light to induce the formation of crosslinks between the bead-bound target and candidate nucleic acid ligands that have bound to the target. In especially preferred embodiments, the UV light has a wavelength of 308 nm, with an intensity of around 500 mW/cm² to photo-activate the 5-BrdU present in the nucleic acid molecules within the pool. UV light sources can be either laser (monochromatic; preferably from an 308 nm XeCl excimer laser) or appropriately filtered lamp sources. The light source may reside on the work surface for direct irradiation; the robotic manipulator can either move the light source to the work station, or the microtiter plate can be moved to the light source. Alternatively, fiber optic light guides or mirrors, or a combination of fiber optics and mirrors, can be used to deliver the light from a source outside the work surface. The total amount of energy delivered to each sample well is individually controlled. In one embodiment of the invention, this control will be achieved using mechanical or liquid crystal shutters placed over the microtiter plate. Such shutters and appropriate lenses/filters will be placed in position via stepper motors and rails mounted above the central magnetic separation module. In another embodiment, the light will be shuttered at the source located off the station and delivered to each well via 96 fiber optic bundles. The fiber bundles can be delivered with a stepper motor and rail mount or by one of the robotic manipulators. Both shuttering methods allow for the simultaneous irradiation of all wells for individually prescribed times. In yet another embodiment, control of UV photo-activation light will be achieved by using a single fiber optic bundle carried by the robotic manipulator. Each well is irradiated separately, one after another, by moving the light bundle to a prescribed distance centered above a well for the desired length of time. The diameter of light from such a bundle is preferably around 7 mm, corresponding to the size of a single microtiter plate well. In preferred embodiments, the total amount of light received by each well is around 0.25 J.

The target beads can then be washed, preferably in buffer comprising one or more of urea, SDS and a chaotropic agent, such as a guanidinium salt, in order to remove all nucleic acid that is not covalently bound to target. In addition, the beads can be incubated at an elevated temperature. Following washing, the bound nucleic acid ligands can be released from target. For protein targets, release can be achieving by treating the target beads with proteinase K, preferably at elevated temperature, to digest the target that has become covalently-linked to the nucleic acid ligands.

Prior to amplification, it is necessary to partition the released candidate nucleic acid ligands from the protease digestion mixture components. Methods for purifying released nucleic acid ligands and method for amplification are described below in the section entitled “Amplification of the Candidate Nucleic Acid Ligands.”

In this application, embodiments of the photoSELEX process in which target is immobilized on solid supports prior to the initiation of photocrosslinking are referred to as the immobilized photoSELEX process.

II. The Automated Solution PhotoSELEX Process

In the automated immobilized photoSELEX process described above, the SELEX target is immobilized on a solid support, such as a paramagnetic bead, before the photocrosslinking step takes place. For a variety of reasons, pre-immobilization of targets, especially proteins, may not, under some circumstances, lead to optimal results in the immobilized photoSELEX process. First, pre-immobilization of protein targets adds an additional preparation step that must be performed before the automated immobilized photoSELEX process can be performed. Second, immobilization may be inefficient, causing target protein to be wasted and leading to less than optimal concentrations of target protein being available during the photoSELEX process. Third, during the immobilization procedure, some protein may be denatured, raising the possibility that the subsequent photoSELEX process will generate nucleic acid ligands to denatured, rather than native, target protein. Finally, the solid supports may scatter or absorb the light used to initiate the formation of crosslinks between the target and the nucleic acid ligands.

The instant invention provides an additional embodiment of the photoSELEX process in which binding and photocrosslinking of target to photocrosslinking nucleic acid ligands in the candidate mixture takes place with the target in solution, rather than immobilized on a solid support as in the immobilized photoSELEX process described above. Following the formation of photocrosslinks, target in solution in the candidate mixture—including target that has formed a nucleic acid-target complex, whether photocrosslinked to nucleic acid or not—is immobilized on a solid support. The solid support is then partitioned from the remainder of the candidate mixture. The solid support can then be washed as described above to remove those nucleic acid ligands that have formed nucleic acid-target complexes but have not become photocrosslinked to the target. In this way, the only nucleic acids that remain on the solid support are photocrosslinking nucleic acid ligands of the target. Following washing, photocrosslinking nucleic acid ligands can then be released from the partitioned solid support by proteolysis, amplified, and optionally used to initiate another round of the photoSELEX process. Because the initial affinity binding and photocrosslinking of nucleic acid ligand to target takes place in solution, this process is referred to as the solution photoSELEX process.

In preferred embodiments of the solution photoSELEX process, the solid support is derivatized with a reagent that interacts with the target. Most preferably, the solid support is derivatized with a reagent or functional group that reacts covalently with the target, but does not react with nucleic acid. For example, for protein targets the solid support can be derivatized with a functional group that reacts with the primary amine groups in the side chains of proteins. One such functional group is the tosyl group well known in the art. After photocrosslinking, the candidate mixture and target are contacted with the tosyl-derivatized solid support. Protein targets, but not nucleic acids, react with the tosyl group, and become covalently attached to the solid support. If a protein target is photocrosslinked to a photocrosslinking nucleic acid ligand, then that photocrosslinking nucleic acid ligand will also be immobilized on the solid support by virtue of its covalent linkage to the protein. By contrast, nucleic acids in the candidate mixture that have not photocrosslinked to target protein will not be covalently immobilized on the solid support. Following blocking of unreacted tosyl groups, stringent, denaturing washing of the solid support can be performed to remove any nucleic acids in the candidate mixture that non-specifically and/or non-covalently associate with the immobilized target. The washing can be performed under conditions that denature nucleic acids, or under conditions that denature proteins, or under conditions that denature both proteins and nucleic acids. In preferred embodiments, the solid supports are washed in a buffer comprising a chaotropic agent, such as a guanidinium thiocyanate, and a detergent.

Alternatively, the solid support is derivatized with a functional group that can react with one of the functional moieties of a bifunctional linker molecule; the other functional moiety of the linker reacts with the target. In this way, the addition of the derivatized solid support and bifunctional linker to the candidate mixture following photocrosslinking leads to the immobilization of target on the solid support. In this embodiment, the bifunctional linker can be either homobifunctional or heterobifunctional.

The solid supports used in the present invention can be of any composition or shape. Preferred solid supports are beads and columns. For columns, the candidate mixture containing the photocrosslinked nucleic acid-target complexes is passed through the derivatized column interior whereby target interacts with the column. Column eluant is discarded, thereby resulting in the partitioning of the solid support from the remainder of the candidate mixture. For beads, partitioning may take place by centrifugation. In particularly preferred embodiments, the solid support comprises paramagnetic beads. Paramagnetic beads can readily be partitioned from the remainder of the candidate mixture by the application of a magnetic field, as described above.

When the target is a protein, particularly preferred embodiments of the solution photoSELEX process use tosyl-activated paramagnetic beads, such as tosyl-activated M-280 beads (available from Dynal Inc.), as the solid support. Following addition of target to the candidate mixture, and initiation of photocrosslinks between the target and photocrosslinking nucleic acid ligands, an aliquot of tosyl-activated beads is added to the candidate mixture. Protein target, including protein target that is found in nucleic acid-target complexes, reacts covalently with the tosyl groups; the beads can be partitioned from the remainder of the candidate mixture by the application of a magnetic field. The beads can then be processed according to the methods described above in order to wash and then release the photocrosslinked photocrosslinking nucleic acid ligands from target protein. For example, following blocking of unreacted tosyl groups, the beads can be washed under stringent, denaturing conditions, then treated with a protease, such as proteinase K, to release the photocrosslinking nucleic acid ligands into solution. The released photocrosslinking nucleic acid ligands are then purified from the protease digestion mixture and amplified as described below in the section entitled “Amplification of the Candidate Nucleic Acid Ligands;” the amplified nucleic acid ligands are then used to initiate another round of the solution photoSELEX process.

In preferred embodiments, the target is coupled to the solid support under conditions that maximize the yield of the coupling reaction. Such conditions may result in the denaturation of protein targets, and/or nucleic acids. If this is the case, then only true photocrosslinking nucleic acid ligands of the target will become coupled to the solid support via their interaction with the photocrosslinked target. Nucleic acid-target complexes that are not photocrosslinked will become disrupted under denaturing conditions, thereby releasing the nucleic acid ligand into solution and preventing such nucleic acid ligands from becoming immobilized on the solid support. Hence, the use of coupling conditions that result in the denaturation of target and/or nucleic acid, further aids in insuring that only true photocrosslinking nucleic acid ligands of the target become immobilized on the solid support. As outlined above, washing the partitioned solid support under denaturing conditions will also remove those nucleic acids ligands that are not photocrosslinked to their target.

By immobilizing target on a solid support after the initiation of photocrosslinking, the present invention achieves a number of desirable results, as detailed below.

First, the amount of preparation that must be completed before performing the automated photoSELEX process is reduced because it is no longer necessary to prepare immobilized target before initiation of the photoSELEX process.

Second, the capture reaction between the target and solid support can be performed under conditions that maximize capture yield. For protein targets, such capture-maximizing conditions might lead to protein denaturation or other alterations in protein conformation. This would be an undesirable result if the protein target was immobilized prior to the initiation of photocrosslinking because it could lead to the generation of photocrosslinking nucleic acid ligands that bind poorly to native protein. Hence, immobilization of proteins prior to photocrosslinking is frequently done under less than optimal capture conditions, leading to some waste of target protein. By contrast, because photocrosslinking takes place in the instant invention after the initiation of photocrosslinking, the potential generation of photocrosslinking nucleic acid ligands to denatured or conformationally-altered protein is no longer a concern, allowing the use capture-maximizing conditions. This is useful when only limited amounts of the target protein are available. In particular, the use of capture-maximizing conditions is especially useful where the target is a tissue. Tissue targets comprise multiple target molecules, some of which are likely to be present at very low concentrations. By using capture-maximizing conditions, the likelihood of generating photocrosslinking nucleic acid ligands to rare target molecules in the tissue target is enhanced. As outlined above, the use of capture conditions that result in protein denaturation, and/or nucleic acid denaturation, further insures that only nucleic acids that are photocrosslinked to the target become immobilized on the solid support.

Third, the effective concentration of protein presented to the candidate mixture is likely to be higher when the target is free in solution, rather than immobilized and constrained on a solid support. Selection of photocrosslinking nucleic acid ligands according to the methods of the instant invention is therefore likely to be more efficient than in embodiments where the target is pre-immobilized. Again, this is likely to be useful where limited amounts of target are present, especially where the target is a tissue comprising both rare and abundant target molecules.

Finally, when photocrosslinking is initiated using solid support-immobilized target, some of the light used to initiate the formation of photocrosslinks is scattered or absorbed by the solid support. In the solution photoSELEX process, photocrosslinking is performed in the absence of solid supports, so no undesirable scattering or absorption of light occurs. As a result, photocrosslinking is more efficient than in embodiments where the target is pre-immobilized.

III. Polymerase Optimization in the Automated PhotoSELEX Process

The photocrosslinking that underpins the photoSELEX process results in the covalent modification of the desirable sequences within the mixture of candidate nucleic acid ligands. In addition, irradiation may induce photodamage to sequences within the photoSELEX candidate nucleic acid ligand mixture. Either of these modifications could conceivably lead to less than optimal replication of the desirable sequences. Therefore, in preferred embodiments, it is desirable to select those DNA polymerases and reverse transcriptases that can most efficiently replicate the modified nucleic acid. In some embodiments, the Klenow exo-fragment of E. coli DNA polymerase, or reverse transcriptases are used to optimize the amplification yield. In other embodiments, a combination of Taq polymerase and Pwo polymerase is used. In still other embodiments, Taq polymerase is used alone.

IV. Maximizing Enrichment in the Automated PhotoSELEX Process

It is possible to push the automated photoSELEX process in the final rounds to an extreme state of enrichment that will facilitate nucleic acid ligand identification. By applying suitably stringent conditions, i.e., maximizing competition among the putative nucleic acid ligands for binding and crosslinking by increasing the number of rounds of the photoSELEX process performed, the enriched pools may be driven to a state of very low sequence complexity. In the most favorable case, the final pools will be dominated by a single nucleic acid sequence that constitutes over 30% of the sequences. The identity of this “winning” nucleic acid ligand can then be easily obtained by sequencing the entire pool, avoiding the need to clone individuals from the pool prior to sequencing. Since the same selection pressures used to evolve the nucleic acid ligands in the first place are used in this final stage, albeit more extreme, the resulting winner should have both good affinity for the cognate target as well as reasonably good efficiency at crosslinking. If necessary, the SELEX process could split into a separate affinity and crosslinking set where these individual pressures could be applied to reduce pool complexity. The two resulting nucleic acid ligands could then be tested for functionality in the assay format—immobilized nucleic acid ligands that capture cognate proteins from solution followed by irreversible crosslinking. It will be appreciated that this method of using suitably stringent conditions to drive a candidate mixture to a state of low sequence complexity can also be used in the affinity SELEX process.

V. Using the Affinity SELEX Process to Produce a Ligand-Enriched Mixture of Nucleic Acids That is Then Used to Initiate the Automated PhotoSELEX Process

In some embodiments of the invention, the automated SELEX process is carried out by first performing one or more rounds of the affinity SELEX process to obtain a ligand-enriched mixture of nucleic acids, then using that ligand-enriched mixture as the initial candidate mixture for the automated photoSELEX process (either the automated solution photoSELEX process or the automated immobilized photoSELEX process). In this way, essentially two serial selections take place: the affinity SELEX process first enriches the candidate mixture for those nucleic acids that have specific binding activity for the target; the photoSELEX process then further selects for those nucleic acids in the ligand-enriched mixture that additionally possess the ability to photocrosslink to the target. This serial selection strategy is based upon the expectation that the initial candidate mixture will contain a number of nucleic acids with affinity for the target, but only a subset of those nucleic acids with affinity will also have the ability to photocrosslink to the target.

By performing serial selections, the probability of obtaining a photocrosslinking nucleic acid ligand is greater than if the automated photoSELEX process is performed alone. Without wishing to be bound by any one theory, it is believed that in some instances, the number of nucleic acids in the initial candidate mixture capable of both binding the target and becoming photocrosslinked to it is likely to be very low. By way of example only, it might be expected that an initial candidate mixture contains 5 to 10 copies of the desired sequence. Because photocrosslinked DNA is sometimes amplified less efficiently than non-photocrosslinked DNA during the PCR process, there is a chance that those few copies of the desired sequence will be lost during the first round of selection if the photoSELEX process is performed alone. By contrast, if the affinity SELEX process is performed first, the desired sequence (which is a subset of those sequences with affinity for the target) will be amplified more efficiently at each round because of the absence of nucleic acid-protein photocrosslinks. As a result, the ligand-enriched mixture of nucleic acids used as the candidate mixture in the automated photoSELEX process may contain many thousands of copies of the particular nucleic acid that can photocrosslink to the target. Inefficiencies in the subsequent amplification of those sequences when photocrosslinked will therefore have less effect on the final outcome of the automated photoSELEX process.

In some embodiments, the initial candidate mixture for the automated photoSELEX process is a ligand-enriched mixture of nucleic acids obtained by performing the affinity SELEX process manually. For example, one or more rounds of the filter affinity SELEX process can be performed in which protein target-nucleic acid ligand complexes are formed in solution, and then are partitioned from the candidate mixture on the basis of their retention on a nitrocellulose filter. Target-nucleic acid complexes and unbound target protein are retained on the filter during vacuum filtration; other nucleic acids are not. The target-nucleic acid complexes can then be recovered from the filter by, for example, heating the filter in eluting buffer. In other embodiments, one or more rounds of the automated or manual bead affinity SELEX process is performed first in order to generate a ligand-enriched mixture of nucleic acids which then serves as the initial candidate mixture for the automated photoSELEX process. In still further embodiments, one or more rounds of the automated or manual plate affinity SELEX process is performed to generate a ligand-enriched mixture of nucleic acids which is then used as the initial candidate mixture for the automated photoSELEX process. It will be appreciated that various combinations of the aforementioned affinity SELEX processes can be carried out in order to prepare a ligand-enriched mixture which will be used as the initial candidate mixture the automated photoSELEX process (either the automated solution photoSELEX process or the automated immobilized photoSELEX process). For example, one round of manual filter affinity SELEX followed by four rounds of the automated bead affinity SELEX process could be used to generate the ligand-enriched candidate mixture.

In preferred embodiments, the candidate mixture of nucleic acids in the initial affinity SELEX process comprises nucleic acids with photoreactive nucleotides that can photocrosslink to the target, even though photocrosslinking is not, by definition, initiated in the affinity SELEX process rounds. If the affinity SELEX process rounds were performed without such photoreactive nucleotides, the resulting candidate mixture would need to be copied with photoreactive nucleotides prior to beginning the photoSELEX process. It is likely that the incorporation of photoreactive nucleotides would change the structure of nucleic acids in the candidate mixture, thereby disrupting the ability of nucleic acid ligands in the candidate mixture to bind to target.

D. Amplification of the Candidate Nucleic Acid Ligands

At the end of each binding and partitioning step in the automated SELEX process (either the affinity SELEX process embodiments, or the photoSELEX process embodiments), the candidate nucleic acid ligands must be released (eluted) from their bound targets and amplified. Methods for release of nucleic acid ligands from bound target are described in detail in the preceding sections e.g., proteolysis for photocrosslinked targets, and NaOH for affinity targets. In preferred embodiments, amplification of released nucleic acid ligands is achieved using the Polymerase Chain Reaction (PCR).

In preferred embodiments, released nucleic acid ligands are partitioned from their targets prior to amplification. In the automated affinity SELEX process using paramagnetic beads and multiwell microtitre plates, this can be accomplished by pulling the beads to the sides of the wells using magnets, and then transferring the solution phase (containing the released nucleic acid ligands) to a new microtitre plate. In the automated photoSELEX process (where nucleic acid ligand are released from their photocrosslinked targets by protease digestion), it is necessary to partition the released nucleic acid ligands from the protease digestion mixture prior to amplification. This can be achieved by dispensing primer-conjugated paramagnetic beads to the protease digestion mixtures after protease digestion is completed. The primers have sequences complementary to the 3′ and/or 5′ fixed sequence regions of the nucleic acid ligands. Released nucleic acid ligands hybridize to the primer, and the primer-conjugated beads can then be washed as described above in order to remove all the protease digestion reagents. Following washing, the nucleic acid ligands can be eluted from the primer-conjugated beads by, for example, the addition of NaOH. The beads can then be pulled to the sides of the wells by magnets, and the solution phase containing the eluted nucleic acid ligands can be transferred to a new microtiter plate for amplification.

Candidate nucleic acid ligands can be single-stranded DNA molecules, double-stranded DNA molecules, single-stranded RNA molecules, or double-stranded RNA molecules. In order to amplify eluted RNA nucleic acid ligands in a candidate mixture, it is necessary first to reverse transcribe the RNA to cDNA. Reverse transcription of eluted RNA ligands can be performed during the automated SELEX process by dispensing the necessary enzymes and buffers to the wells on the work station containing the eluted ligand. The reaction mixtures are then incubated on the work station at a temperature that promotes reverse transcription. The resulting cDNA molecules are then amplified as described in the following paragraphs and in the section entitled “Amplification, Transcription, and Purification of RNA Ligands.”

In preferred embodiments, amplification of DNA molecules is carried out using the polymerase chain reaction (PCR) with primers that are complementary in sequence to the 5′ and 3′ fixed sequence regions of the candidate nucleic acid ligands. Preferably, PCR is carried out with reagents and conditions that prevent the formation of high molecular weight artifacts of the amplification process, termed “parasites.” Parasites sometimes form during the automated SELEX process when the amplified candidate mixture of each round is not size fractionated prior to initiating the next round of the SELEX process. While not wishing to be bound by any particular theory, it is believed that parasites result from rare mispriming events that occur during PCR. These mispriming events are believed to occur when rare candidate nucleic acid ligands contain a sequence in their random regions that is complementary in sequence to the 3′ fixed sequence. If the 3′ fixed sequence folds back over this complementary sequence in the random region, a self-priming intramolecular duplex may form. This structure can be extended by Taq polymerase to form a longer product during PCR amplification. Alternatively, the 3′ fixed sequence of another candidate nucleic acid ligand can form an intermolecular duplex with the complementary sequence in the random region, and the 3′ end of the former candidate nucleic acid can be extended by Taq polymerase to form a longer product. A series of either of these events will produce parasites with a variable number of repeats. Once these parasites have formed, they will anneal promiscuously with other nucleic acids, including the correct products, leading to the formation of ever-larger parasites through 3′ end extension. As parasites grow, they contain more and more primer binding sites, allowing them to be efficiently amplified during the PCR process at the expense of bona fide nucleic acid ligands for primer. In the most extreme cases, nucleic acid ligand products are sometimes eliminated from the candidate mixture of nucleic acid ligands that contains a parasite.

In preferred embodiments, the likelihood that parasites will form is reduced by adding sequences with melting temperature (Tm) values lower than the PCR annealing temperature to the 5′ termini of the PCR primers. At the annealing temperature, hybridization of these sequences to their complements is unstable, whereas the primers anneal to the fixed sequence regions of the candidate nucleic acids. These unstable sequences that are added to the 5′ end of primers are referred to as “tails,” and the resulting primers are referred to as “tailed primers.” For example, PCR can be performed with one primer linked to a tail sequence ATATATAT ((AT)₄), and the other linked to the tail sequence TTTTTTTT ((T)₈). The correct PCR product will have ATATATAT on the 3′ terminus of one strand and AAAAAAAA on the 3′ terminus of the other strand. At a typical PCR annealing temperature of 60° C., the tail sequences AAAAAAAA and ATATATAT will not anneal intra- or intermolecularly to the random regions of candidate nucleic acid ligands that fortuitously contain the complements of those sequences. It will be recognized by those skilled in the art that other sequences with low Tm may also be used. In preferred embodiments, the initial candidate mixture also has unstable tail sequences at its 5′ and 3′ ends to minimize the chance that parasites form during the first PCR cycle. For example, if the primers described above are used, then the initial candidate mixture could have the sequence ATATATAT at its 5′ end, and the sequence AAAAAAAA at its 3′ end. An example of such a tailed candidate mixture is provided in Example 3 below. Methods for designing and using tailed primers are described in great detail, along with other methods for preventing parasite formation, in U.S. patent application Ser. No. 09/616,284, filed Jul. 14, 2000, and in U.S. patent application Ser. No. 09/815,171, filed Mar. 22, 2001, each of which is entitled “Method and Apparatus for the Automated Generation of Nucleic Acid Ligands” and each of which is incorporated by reference in its entirety.

In some embodiments, one or both of the primers used for amplification of the DNA molecules (which molecules are either DNA ligands or cDNA formed by the reverse transcription of RNA ligands) are also conjugated to a molecule useful for capture of the strand(s) into which the primer is incorporated during PCR. For example, one or both primers can be conjugated to biotin; PCR products that have incorporated the biotin primer can be partitioned using streptavidin-conjugated solid supports, such as paramagnetic beads. Alternatively, the primer can bear a unique capture sequence, allowing paramagnetic beads conjugated to a complementary nucleic acid to partition PCR products that have incorporated the primer. Using these methods, it is possible to partition double-stranded PCR products from other components of the amplification reaction mixtures. Furthermore, by incorporating the capture molecule into only one primer it is possible to perform strand separation of the partitioned PCR products. For example, if a biotin-labeled 3′ primer (the primer that hybridizes to the 3′ end of a candidate nucleic acid) is used during PCR of a DNA candidate mixture, it will incorporated into the antisense strand of the product. Double stranded PCR products can then be partitioned from the PCR reaction mixture using streptavidin beads, and the beads can be washed if required. The sense strand (non-biotinylated) can then be eluted into the solution phase, for example by using NaOH. The beads can be held to the sides of the well and the solution phase containing the sense strand can be removed by the robot for use as the enriched candidate mixture in the next round of the automated SELEX process.

In embodiments in which PCR reactions are monitored using SYBR Green 1 dye (see below in the section entitled “Calculation of the Amount of Eluted Nucleic Acid Ligand in Each Amplification Mixture”), the use of a biotinylated primer also allows the sense strand to be partitioned from the dye and the PCR reaction mixture in order to begin the next round of the SELEX process.

In preferred embodiments of the automated photoSELEX process, the nucleic acid ligands released from target are amplified with the appropriate photoreactive nucleotides in the PCR reaction mixture e.g. by including 5-BrdU triphosphate (5-BrdUTP) along with dATP, dCTP, and dGTP. In other embodiments, PCR is carried out with non-photoreactive nucleotides and the antisense PCR products are isolated according to one of the methods described above e.g., by using a biotinylated 5′ primer that becomes incorporated into the sense strand during PCR, capturing the double-stranded PCR products on streptavidin-conjugated beads, washing the beads, and then eluting the antisense strand from the beads. The antisense strands can then serve as the template for the polymerization of new sense strands in the presence of photoreactive nucleotides.

E. Amplification, Transcription, and Purification of RNA Ligands

For RNA ligands, the antisense strands of the amplified cDNA molecules must be partitioned and transcribed to regenerate the pool of candidate RNA ligands for the next round of the automated SELEX process. This can be achieved by using primers in the amplification step that contain sites that promote transcription, such as the T7 polymerase site. These primers become incorporated into the antisense strands of the amplification products during the PCR step. In addition, the PCR primer that becomes incorporated into the sense strand preferably contains biotin, allowing the non-biotinylated antisense strand to be eluted from the biotinylated sense strand following partitioning of dsDNA from the amplification reaction mixture using streptavidin beads. The eluted antisense strand (containing the T7 polymerase site at its 3′ end) can then be transcribed by T7 polymerase using an additional primer that binds to the 3′ end of the antisense strand and contains an initiation site for T7 RNA polymerase.

In some embodiments, newly transcribed RNA ligands are purified from their amplified cDNA transcription templates before beginning the next round of the automated affinity SELEX process or automated photoSELEX process. This can be done using a set of paramagnetic beads to which primers complementary to the 3′ fixed region of the RNA ligands are attached. When these primer beads are added to the transcribed amplification mixture, the newly transcribed full length RNA ligands hybridize to the bead-bound primer, whereas the amplified double-stranded DNA molecules remain in solution. The beads can be separated from the reaction mixture by applying a magnetic field to the wells and aspirating the liquid in the wells, as described above. The beads can then be washed in the appropriate buffer at a preselected temperature, and then the RNA ligands may be eluted from the beads by heating in an elution buffer (typically dH₂O). Finally, the beads may be partitioned from the eluted candidate RNA ligands.

The amount of primer bead added determines the amount of RNA ligand that is retained in the wells. Therefore, the amount of RNA ligand that is used in the next round of the automated SELEX process can be controlled by varying the amount of primer bead that is added to the amplification mixture. The amount of RNA ligand that is to be used can be determined through quantitation of the amount of PCR product (see below). A predetermined amount of the amplified mixture is then used in the next round of the automated SELEX process.

F. Calculation of the Amount of Eluted Nucleic Acid Ligand in Each Amplification Mixture

In certain embodiments, it may be important to measure the amount of candidate nucleic acid ligand eluted from the target before beginning the next round of the automated SELEX process. Such measurements yield information about the efficiency and progress of the selection process. The measurement of eluted nucleic acid ligand—which serves as template for the amplification reaction—can be calculated based on measurements of the amount of amplification product arising out of each PCR reaction.

In preferred embodiments, the amount of PCR product is measured using a fluorescent dye that preferentially binds to double stranded DNA (dsDNA). One suitable dye is SYBR Green I, available from Molecular Probes, Inc., Eugene, Oreg. The fluorescence signal of this dye undergoes a huge enhancement upon binding to dsDNA, allowing dsDNA to be detected in real time within the PCR reaction mixture, without fluorescent signal contribution from the single stranded primers. Methods for the use of SYBR Green I in quantitative PCR applications are described in Schneeberger, et al., PCR Meth. Appl. 4: 234 (1995), incorporated herein by reference in its entirety. Preferably, SYBR Green I is included within the PCR reaction mixture. The progress of the PCR reaction can either be monitored in real-time, or it can be monitored periodically after a predetermined number of cycles have taken place.

U.S. patent application Ser. No. 09/815,171, filed Mar. 22, 2001, U.S. patent application Ser. No. 09/616,284, filed Jul. 14, 2000, U.S. patent application Ser. No.09/356,233, filed Jul. 16, 1999, and U.S. patent application Ser. No. 09/232,946, filed Jan. 19, 1999, each of which is entitled “Method and Apparatus for the Automated Generation of Nucleic Acid Ligands” describe additional fluorescence-based methods for the quantitation of nucleic acids during the automated SELEX process, including methods using primer labeled with fluorescent (F) groups and quenching (Q) groups, and also including methods that use the TaqMan™ probe PCR system available from Roche Molecular Systems.

The current invention contemplates the use of fluorometry instruments that can monitor the fluorescence emission profile of the reaction mixture(s) on the work station during thermal-cycling in the presence of fluorescent dyes, or the aforementioned F/Q primers. Suitable instruments contemplated comprise a source for excitation of the fluorophore, such as a laser, and means for measuring the fluorescence emission from the reaction mixture, such as a Charge Coupled Device (CCD) camera. Appropriate filters are used to select the correct excitation and emission wavelengths. Especially preferred embodiments use a fluorometry instrument mounted on an optically-transparent cover that can be placed over the wells on the work station by the robotic manipulator. When placed over the wells and then covered with a light shield, this fluorometry cover can capture an image of the entire array at pre-selected intervals. The computer interprets this image to calculate values for the amount of amplified product in each well at that time. At the end of the amplification step, the robotic manipulator removes the light shield and fluorometry cover and returns them to a storage station on the work surface.

In alternative embodiments, quantitative PCR can performed using a commercially available instrument located either on the work surface or off the work surface. Microtitre plates can be moved to this machine either by the robotic manipulator if it is on the work surface, or by the operator if located off the work surface. In especially preferred embodiments, quantitative PCR is performed using the ABI 5700 GeneAmp thermal cycler (Applied Biosystems, Inc.) and SYBR Green I dye.

In preferred embodiments, measurements of PCR product quantity are used to determine a value for the amount of eluted nucleic acid ligand introduced as template into the amplification reaction mixture. This can be done by comparing the amount of amplified product with values stored in the computer that were previously obtained from known concentrations of template amplified under the same conditions. In other embodiments, the automated SELEX process apparatus automatically performs control PCR experiments with known quantities of template in parallel with the candidate nucleic acid amplification reactions. This allows the computer to re-calibrate the fluorescence detection means internally after each amplification step of the automated SELEX process.

The value for the amount of candidate nucleic acid ligand eluted from the target (derived from the measurement of the amount of amplified product) is used by the computer to make optimizing adjustments to any of the steps of the automated SELEX process method that follow. For example, the computer can change the selection conditions in order to increase or decrease the stringency of the interaction between the candidate nucleic acid ligands and the target. The computer can also calculate how much of the nucleic acid ligand mixture and/or target protein should be used in the next automated SELEX process cycle. In the automated solution photoSELEX process embodiment, the computer can calculate the appropriate solution protein concentration to be used in each round. In embodiments using primer beads (see the sections above entitled “Amplification of the Candidate Nucleic Acid Ligands” and “Purification of Newly-Transcribed RNA Ligands”), the computer uses this information to determine the amount of primer bead suspension to be added to each well on the work station(s). Similarly, the computer can change the conditions under which the candidate nucleic acid ligands are amplified. All of this can be optimized automatically without the need for operator intervention.

The methods provided herein allow quantitation of PCR product in each parallel PCR reaction. This information can also be used to determine when an individual PCR reaction has incorporated all of the free primer initially added. Reactions identified in this way can be terminated in order to prevent the unproductive cycling that can lead to formation of parasites as described in U.S. patent application Ser. No. 09/616,284, filed Jul. 14, 2000, and in U.S. patent application Ser. No. 09/815,171, filed Mar. 22, 2001, each of which is entitled “Method and Apparatus for the Automated Generation of Nucleic Acid Ligands.” In some embodiments, PCR reactions can be carried out for a predetermined number of rounds, and then the amount of primer incorporated into the reaction products is determined, preferably through the use of a dye, such as SYBR Green I, that binds to dsDNA. Individual PCR reactions that are substantially complete can then be removed from the thermal cycler; reactions that are not yet substantially complete can be cycled for an additional number of rounds. Alternatively, reactions that are substantially complete can be stopped by the addition of a terminating agent, such as EDTA. This process can be repeated until all reactions are substantially complete. By way of example only, PCR reactions can be carried out for 10 rounds initially; at the end of those first 10 rounds, quantitation will reveal those reactions that should be removed from the cycler, and those that must continue to cycle. The reactions that have yet to progress to completion can then be cycled for an additional 5 rounds, and the quantitation process repeated. Additional ways for preventing the unproductive thermal cycling in the absence of free primer that can lead to parasite formation are described in U.S. patent application Ser. No. 09/616,284, filed Jul. 14, 2000, and in U.S. patent application Ser. No. 09/815,171, filed Mar. 22, 2001, each of which is entitled “Method and Apparatus for the Automated Generation of Nucleic Acid Ligands.”

G. Analysis of the Aptamers Produced by the Automated PhotoSELEXProcess

Performance of the automated SELFX process according to any of the embodiments described herein leads to the production of an enriched pool (candidate mixture) of nucleic acid ligands for each target i.e., for 96 targets, 96 pools are produced. As a preliminary step in the evaluation of the aptamers, it is preferable to perform activity assays for each pool. Preferably, the assays measure a value for the apparent interaction affinity. For photocrosslinking nucleic acid ligands, the assay also measures a value for the fraction of nucleic acid crosslinked to target at saturating target protein concentration. Non-limiting, exemplary methods for determining aptamer and photocrosslinking nucleic acid ligand activities are provided in Examples 9 and 10 below.

In order to further characterize the individual aptamers or photocrosslinking nucleic acid ligands in a single pool, those nucleic acid molecules are preferably cloned and then sequenced. Because the automated affinity SELEX and photoSELEX processes described herein can rapidly produce formidable numbers of such nucleic acid ligands for characterization, it is necessary to have a robust and high-throughput strategy for the cloning and sequencing. Non-limiting, exemplary methods for amplifying and cloning pools of nucleic acid ligands are provided in Example 12.

In some embodiments, a pool of nucleic acid ligands is only cloned and sequenced if the aggregate binding activity of that pool (including the photocrosslinking activity for photocrosslinking nucleic acid ligand pools) exceeds a predetermined value. For example, a pool of photocrosslinking nucleic acid ligands may be cloned and sequenced only if the fraction of nucleic acids in that pool that can photocrosslink to target protein exceeds 0.05.

For each pool of nucleic acid ligands, preferably the primary sequence of 24-48 clones is determined. Sequences can be aligned to identify common features using Clustal analysis and analyzed by visual inspection. Isolates that are most heavily represented (many isolates with the same sequence) or shared a common sequence motif can be chosen for further characterization. Plasmids from the sequencing procedure containing inserts with the chosen sequences can then be used as templates for amplification of the inserts by PCR to produce individual aptamers for analysis. The PCR reactions are preferably done with biotinylated antisense primer for streptavidin bead purification of the aptamer (sense) strand as described above. The aptamers from each active library can then be tested for activity to their cognate proteins described in the examples below.

H. Combinations of the Core Methods Provided Above

It will be appreciated by those skilled in the art that there are many combinations of the core methods provided in this application that are suitable for the generation of photocrosslinking and non-photocrosslinking nucleic acid ligands. It is expressly contemplated that the skilled artisan treat the various core methods as modular components that can be assembled in a variety of combinations. The highly-parallel nature of the automated affinity SELEX process and the automated photoSELEX process—the ability to process 96 or more samples in a single experiment—allows one skilled in the art routinely to experiment with various combinations of the automated affinity SELEX process, the manual affinity SELEX process, the automated solution photoSELEX process, and the automated immobilized photoSELEX process. Such routine experimentation allows the skilled artisan rapidly to determine the most favorable selection conditions for a particular application. In addition, it will be appreciated that although the methods described herein are specifically designed to enable high-throughput automation of the SELEX process, they can still be performed manually. The descriptions of such combinations that follow in the Examples section below illustrate a number of potential combinations and are not to be interpreted as limiting the scope of the invention in any way.

### EXAMPLES

### Example 1

### Apparatus for Performing the Automated Affinity SELEX Process

FIGS. 1-4 show various views of an embodiment of an apparatus for performing automated SELEX according to the present invention. This embodiment is based on the Tecan™ (Cavro) robot system. It should be noted, however, that other robotic manipulation systems may also be used in the present invention, such as the MultiPROBE™ system (Packard), the Biomek 200™ (Beckman Instruments). Each view shows the apparatus during the PCR amplification stage of the automated SELEX process.

In FIG. 1, a perspective view of this apparatus is shown. The system illustrated comprises a work surface 71 upon which the work station 72 is located (work station is partially obscured in this perspective view but can be seen in FIGS. 2, 3, and 4 as feature 72). The pipetting tool 74 and the aspirator 75 are attached to a central guide rail 73 by separate guide rails 77 and 78 respectively. The pipetting tool 74 can thus move along the long axis of guide rail 77; guide rail 77 can then move orthogonally to this axis along the long axis of central guide rail 73. In this way, the pipetting tool 74 can move throughout the horizontal plane; the pipetting tool can also be raised away from and lowered towards the work surface 71. Similarly, aspirator 75 is attached to guide rail 78, and guide rail 78 is attached to central guide rail 73 in such a way that aspirator 75 can move in the horizontal plane; aspirator 75 can also move in the vertical plane.

The fluorometry cover 76 is attached to guide rail 79 viabracket 710. Bracket 710 can move along the vertical axis of guide rail 79, thereby raising fluorometry cover 76 above the work station 72. When fluorometry cover 76 is positioned at the top of guide rail 79, then guide rails 77 and 78 can move underneath it to allow the pipetting tool 74 and the aspirator 75 to have access to work station 72. In this illustration, the fluorometry cover 76 is shown lowered into its working position on top of the work station 72.

Fluorometry cover 76 is attached to a CCD camera 711a and associated optics 711b. A source of fluorescent excitation light is associated with the cover 76 also (not shown). When positioned on top of the work station 72, the cover 76 allows the CCD camera 711a to measure fluorescence emission from the samples contained on the work station 72 during PCR amplification. For clarity, the light shield which prevents ambient light from entering the fluorometry cover—is omitted from the drawing. When PCR amplification is finished, fluorometry cover 76, with attached CCD camera 711a and optics 711b, is simply raised up guide rail 79.

Also not visible in this view, but visible in FIGS. 2 and 4, is the heated lid 91, which is resting on top of the work station 72 underneath the fluorometry cover 76. The work surface 71 also comprises a number of other stations, including: 4° C. reagent storage stations 712, a −20° C. enzyme storage station 713, ambient temperature reagent storage station 714, solution discard stations 715, pipette tip storage stations 716 and archive storage stations 717. Pipetting tool 74 is also associated with a gripper tool 718 that can move objects around the work surface 71 to these various storage locations. Lid park 719 (shown unoccupied here) is for storage of the heated lid (see FIGS. 3 and 4).

FIG. 2 shows the instrument of FIG. 1 in a plan elevation view. Each element of the instrument is labeled with the same nomenclature as in FIG. 1.

FIG. 3 is a front elevation view of the instrument in FIG. 1. Note that each element of the instrument is labeled with the same nomenclature as in FIG. 1 and FIG. 2. Note also that in this view, it can be seen that work station 72, and chilled enzyme and reagent storage stations 712 are each associated with shaking motors 92. Operation of these motors keeps the various reagents mixed during the automated SELEX process. The motors 92 are each under computer control, and can be momentarily stopped to allow reagent addition or removal, as appropriate, to the receptacle that is being agitated. Also visible in this view is heated lid 91 which is resting on top of work station 72 to insure uniform heating of the samples.

FIG. 4 is a right side elevation view of the instrument shown in FIGS. 1, 2, and 3. Every element of the instrument is labeled with the same nomenclature as in FIGS. 1, 2, and 3.

FIG. 5 illustrates another embodiment of an instrument work surface 50 in plan view. The gripper tool 51 is shown in the park position. Magnet slider 52 is shown in the extended position such that the individual magnets 53 are engaged with work station 54.

### Example 2

### Apparatus for Performing the Automated PhotoSELEX Process and the Automated Affinity SELEX Process

FIG. 6 illustrates schematically in perspective view another embodiment of the work surface for performing the automated affinity SELEX process and the automated photoSELEX process (including both the automated immobilized photoSELEX process and the automated solution photoSELEX process). In this case, the work surface 60 comprises the following elements (shown schematically and not to scale):

a) an “enzymeRack” 61 comprising 1.7 mL tubes stored at −20° C;

b) a “targetRack” 62 for the storage of target proteins (either in solution or conjugated to paramagnetic beads) comprising a 96 well 1.0 mL plate incubated at 4° C. on a shaker;

c) a “dilutionrack” 63 for the preparation of dilutions of target proteins, comprising a 96 well 1.0 mL plate incubated at 4° C. on a shaker;

d) a rack of 7 mL tubes 64 (“Falcon7Rack”) on a shaker for the storage of tosyl, primer, and streptavidin beads;

e) a rack of 15 mL tubes 65 (“Falcon15Rack”) for the storage of buffer solution;

f) three racks of 0.2 mL pipette tips 66a-c (“tipRack1-3”);

g) two liquid waste containers 67a-b;

h) a tip waste container 68;

i) a rack of 1.7 mL tubes 69 (“eppiRack”);

j) a “selectionModule” 610 comprising a 96 well plate with 0.3 mL wells, a shaker, adjacent to a magnet slider 611a. The magnet slider 611a comprises a computer-controlled stepper motor linked to six bars, each bar having eight permanent magnets spaced along its length such that when the bar is inserted between the wells of plate on the selectionModule, each well is adjacent to at least one magnet. The selectionModule is the site where candidate nucleic acid ligands are contacted with target. The magnet slider 611a and the selectionModule 610 are shown in more detail in FIG. 7 and FIG. 8.

k) a PCR rack (“pcrRack”) 612 comprising a 96 well 0.2 mL optical plate. Nucleic acid ligands eluted from target in the selection module are transferred by the robot to the pcrRack; the pcrRack is then transferred manually to a GeneAmp 5700 thermal cycler located off the work surface.

1) a “purificationModule” 613 comprising the same elements as the selection module, and is also adjacent to a second magnet slider 611b. The purificationModule is where the aptamer (sense) strands are purified from PCR reaction mixtures following return of the pcrRack to the work surface.

3m) a “dnaArchiveRack” 616 for the archival storage of DNA at the end of each round of the automated SELEX process;

n) a laser tool 617 for the irradiation of each well of the selectionModule with 308 nm light from an excimer laser in the automated photoSELEX process. The robotic manipulator (not shown in this plan view) grasps the laser tool in the automated photoSELEX process and uses it to irradiate each well on the selectionModule with 308 nm light. The light is supplied by an excimer laser source located off the work surface and connected to the laser tool 617 via a fiber optic bundle.

Also shown in FIG. 6 is the central guide rail 618 connected to the work surface 60 by two vertical supports 619. Guide rails 620a and 620b can move horizontally along the central guide rail 618. Pipetting tools 621a and 621b are attached to guide rails 620 in such a way that the pipetting tools can move in the vertical axis through guide rails 620. In addition, each pipetting tool 621a and 621b can move along guide rail 620 orthogonally to the axis of the central guide rail. The pipetting tools can add and remove liquid from the individual work stations or modules; liquid that is to be discarded (e.g., wash solutions) can be ejected into liquid waste containers 67a and 67b.

FIG. 7 illustrates a right side elevation view of the selectionModule and magnet slider 611a of FIG. 6 (the elements in FIG. 7 are labelled as in FIG. 6). Note that the elements are not shown drawn to scale. A 96 well plate 70 sits on top of an aluminium block 71, which in turn sits on the top of a Peltier element 72. The Peltier element 72 sits on top of a copper heat exchanger 73 connected to a water hose 74 through which cooling water may be pumped. The copper heat exchanger 73 sits on top of a shaker assembly 75. An off center cam 76 converts the motion of motor 77 into a gyratory motion for shaking the contents of plate 70. Rubber standoffs 78 dampen the motion. Adjacent to the plate 70 is the magnet slider assembly 611a. A series of 6 bars 710 (only one bar visible in this view) each comprise 8 permanent magnets 711 spaced along the length of each bar 710 at intervals such that when the bar 710 is inserted between the wells of plate 70, each well on the plate is adjacent to at least one magnet. Magnet bars 710 are inserted and removed from between the wells of plate 70 in the following way: motor 712 is connected via a pulley system 713 to a lead screw 714. The bars 710 are connected to lead screw 714 via a threaded carriage 715. When the motor 712 is activated by the computer (not shown), lead screw 714 turns, and threaded carriage 715 moves along the length of lead screw 714; the direction of motion is determined by the direction in which the motor 712 turns. Shaker assembly 75 and magnet slider assembly 79 are located on work surface 60.

FIG. 7 also illustrates the laser tool 617 used to irradiate the individual wells to initiate photocrosslinking during the automated solution photoSELEX process. Laser tool 617 comprises a collimating lens 718 in housing 719. A fiber optic cable 720 supplies laser light to the collimating lens 718. The housing 719 also comprises an embedded pipette tip 721; this allows the laser tool 717 to be picked up by pipetting tool 621b.

FIG. 8 illustrates a plan elevation view of the instrument depicted in FIG. 6 and FIG. 7. The individual elements on the work surface 60 are named and labelled according to FIGS. 6 and 7. Magnet sliders 611a and 611b are illustrated also; in this view the 6 magnet bars 710, each bar 710 comprising 8 permanent magnets 711 are also visible.

In this example and in example 1 described above, the operation and monitoring of the robot is controlled by computer. In preferred embodiments, the software that drives the robot is written in an object-oriented fashion, whereby each mechanical or electronic device on the robot is represented by a corresponding object in the software (the terms in quotation marks above, such as “pcrRack,” are examples of such objects). Wells for holding liquid, 96-well plates, lids, tips, manipulators, or any other physical or conceptual object on the robot may also be represented by corresponding objects in the software. In particularly preferred embodiments, the software that drives the robot is written in Java. Particular devices on the robot may be driven by software written in C++ or C, for which existing libraries of method calls are already available. These software libraries are interfaced with the central software driving the robot. In preferred embodiments, software “scripts” may be written to run any desired protocol, or sequence of moves on the robot. These scripts may be written and compiled in separate files from the software which runs the robot. In particularly preferred embodiments, these scripts may be run in simulation mode, in which scripts may be tested for errors without actually running the robot.

### Example 3

### Preparation of a 30N7.1 Candidate Mixture

Tailed 30N7.1 candidate mixture has the following structure in which N is a 30 base long randomized region of A, G, C, or T and in which all T residues are 5-BrdU:5′ATATATATGGGAGGACGATGCGG[N]₃₀CAGACGACGAGCGGGAAAAAAAAA 3′ SEQ. ID. NO 70

The underlined bases comprise the tails that prevent high molecular weight parasites of the amplification process from disrupting the automated SELEX process, as described in U.S. patent application Ser. No. 09/616,284, filed Jul. 14, 2000, and in U.S. patent application Ser. No. 09/815,171, filed Mar. 22, 2001, each of which is entitled “Method and Apparatus for the Automated Generation of Nucleic Acid Ligands.” Synthesis of tailed 30N7.1 candidate mixture is achieved by PCR amplification in the presence of 5-BrdUTP of the following non-BrdU modified template (AB)₂-30N7.1 (obtained as purified synthetic oligonucleotide from Operon, Inc.) (B represents Biotin-ON™ from Clontech Laboratories, Palo Alto, Calif.):

5′ABABTTTTTTTTTCCCGCTCGTCGTCTG[N]30OCCGCATCGTCCTCCCATATATAT3′ SEQ. ID. NO 71

The template is amplified using the following primers:

5′ ATATATATGGGAGGACGATGCGG 3′ (AT)4-5P7 SEQ. ID. NO 72 5′ ABABTTTTTTTTTCCCGCTCGTCGTCTG 3′ (AB)2-(T)8-3P7.1 SEQ. ID. NO 73

A large scale amplification mixture is set up in a volume of 50 mL of 1X SQ8 PCR Buffer [40 mM Tris-HCl, pH 8.3, 50 mM KCl, 2.5 mM MgCl₂, 0.2 mM each of DATP, dCTP, dGTP, 5-BrdUTP, and 1XSYBR Green I (a 1:10,000 dilution of manufacturer stock)], with 6 nmoles of gel-purified template, 24 nmoles of (AB)₂-(T)₈-3P7.1, 30 nmoles of (AT)₄-5P7 and AmpliTaq DNA polymerase. 125 μL aliquots of the amplification mixture are transferred to 96-well plates and amplified for 6-10 cycles of 96° C. for 20 seconds/75° C. for 60 second amplification, the individual reactions are pooled and ethanol precipitated. The product is resuspended, mixed with a 1.5 molar excess of streptavidin, heated to denature the DNA, and run on a denaturing polyacrylamide gel. The biotinylated DNA strand binds to the streptavidin and so migrates to a higher position on the denaturing gel during electrophoresis than the non-biotinylated DNA strand. The non-biotinylated strand is purified from the gel by standard methods.

Tailed 40N7. 1 candidate mixture is also produced according to this protocol except that the template has a 40 base long randomized region.

### Example 4

### The Affinity SELEX Process Using Nitrocellulose Filter Partitioning

Target protein and DNA library were equilibrated in 100 μL 1×FSB (Filter Selection Buffer (40 mM HEPES, pH 7.5, 111 mM NaCl, 5 mM KCl, 1 mM MgCl₂, 1 mM CaCl₂, 0.001% HSA) for 30 minutes at room temperature filtered under vacuum through a nitrocellulose membrane prewet with 1 mL FSB, and washed once with 5 mL FSB. DNA was recovered from the filter by heating the filters for 5 minutes at 70° C. in 400 μL FEB (Filter Elution Buffer: 50% phenol, 4M urea). 200 μL dH20 was added to the eluant and the aqueous phase containing the DNA was collected after centrifugation and extracted once with 400 μL CHCl₃ to remove trace phenol. DNA was recovered from the aqueous phase by EtOH precipitation and resuspended in 100 μL dH2O. 25 μL of 5×SQ8 PCR Buffer+primer+Taq [200 mM Tris-HCl, pH 8.3, 250 mM KCl, 12.5 mM MgCl₂, 1 mM each dATP, dCTP, dGTP, 5-BrdUTP, 5×SYBR Green 1, 5 μM each (AT)₄-5P7 and (AB)₂-(T)₈-3P7.1, 0.25 U/μL Taq DNA Polymerase] was added to the DNA, and the amplification mixture was cycled 96° C., 15 seconds, 75° C., 60 seconds for 20 cycles in an ABI 5700. PCR is done with a biotinylated 3′ primer allowing capture of the product by streptavidin. 25 μL Pierce MagnaBind-SA (streptavidin) beads (5 mg/mL) were prepared by washing twice with 20 mM NaOH, once with 1×Selection Buffer (SB) (40 mM HEPES, pH 7.5, 111 mM NaCl, 5 mM KCl, 1 mM MgCl₂, 1mM CaCl₂, 0.05% TWEEN-20) and resuspending in 25 μL 5M NaCl. 25 μL SA beads were added to 100 μL PCR product and incubated for 5 minutes at 20° C. Beads were washed 3 times with 100 μL 1×SB by pulling the beads aside with a magnet, replacing the buffer, and resuspending the beads. The non-biotinylated (sense) strand of the captured PCR product was eluted from the beads by removing the wash buffer, adding 80 μL 20 mM NaOH, resuspending the beads, and incubating for 1 minute at 20° C. The eluent containing the DNA was recovered and neutralized with 20 μL 80 mM HCl. Half of the DNA was archived and the other half was diluted 2×by adding 50 μL dH2O. A 1 μL aliquot of the archived DNA was analyzed for size homogeneity by 8% denaturing PAGE. This completed one round of the filter affinity SELEX process. The DNA and protein concentrations in round 1 were 1 μM. In subsequent rounds, the DNA concentrations were 100-200 μM and the protein concentrations were lowered in response to a high selection signal. The selection signal was measured during the PCR reaction each round with SYBR Green 1 by standard quantitative PCR techniques. Selection signals ranged from le10-le12 copies DNA, limited on the lower end by protein-independent retention of DNA by the filter. Protein concentrations were lowered 10-fold when selection signal exceeded le11 copies.

### Example 5

### The Manual Solution PhotoSELEX Process

A tailed 30N7.1 5-BrdU candidate mixture was prepared according to the method in example 3. One round of the manual filter affinity SELEX process was then performed according to the method provided in example 4 above using six experimental and two control preparations. The experimentals were selections for crosslinkers to human neutrophil elastase (hNE), HIV-1_{MN} gp120, human IgE, human L-selectin, human platelet-derived growth factor (PDGF), and human alpha-thrombin. The positive control was a selection to human basic fibroblast growth factor (bFGF)-a random library was spiked with 10⁶ copies of a previously-selected photocrosslinking nucleic acid ligand to this target (0615). The negative control contained no protein target.

Ten μl of the PCR product from each affinity SELEX process was amplified by 15 cycles of PCR under the same conditions described above, except that the sense-strand primer, which is incorporated into the aptamer, was radiolabeled at its 5′ end with ³²P, to allow the aptamer to be monitored during the process.

The eight radiolabeled, amplified libraries were then purified by first capturing on 25 μl of 5 mg/ml Magna-bind Streptavidin paramagnetic beads (Pierce), and incubating for 5 minutes at room temperature in a HybAid 96-well multi-plate. The beads were pulled to the side of the wells using a Dynal 96-well magnet plate, aspirated, then alternately washed and resuspended in 1×Solution SELEX Buffer (SSB) (50 mM HEPES pH7.5, 111 mM NaCl, 5 mM KCl, 1 mM CaCl₂, 1 mM MgCl₂, 0.01% Tween-20). The aptamer strand was eluted from the captured double-stranded DNA by denaturation in 80 μl 20 mM NaOH. The eluate was neutralized by addition of 20 μl 80 mM HCl, then buffered by the addition of 20 μl 5×SSB.

After removing 20 μl of the preparation for an archive, the remaining DNA was transferred to a HybAid 96-well multiplate. Target proteins were added at the concentrations (nM protein) indicated in Table 1, and allowed to equilibrate for 5 minutes. The DNA-protein mixtures were irradiated at 308 nm by a XeCl excimer laser. The light was delivered through a fiber optic probe manipulated by the robotic manipulator. The total amount of light was 0.25 J delivered in a beam of 0.2 cm², for an intensity of 1.25 J/cm².

TABLE 1
Target concentration.

SELEX round

| Target | 1 | 2 | 3 | 4 | 5 | 6 |
|---|---|---|---|---|---|---|
| hNE | 500 | 100 | 50 | 50 | 25 | 25 |
| gp120_{MN} | 500 | 250 | 125 | 125 | 6.25 | 3.125 |
| IgE | 500 | 500 | 500 | 500 | 100 | 50 |
| L-selectin | 500 | 500 | 500 | 500 | 500 | 500 |
| PDGF | 500 | 100 | 50 | 50 | 25 | 25 |
| Thrombin | 500 | 500 | 500 | 500 | 100 | 20 |
| bFGF | 500 | 210 | 100 | 20 | 10 | 5 |

Units are nM protein.

DNA crosslinked to protein was then partitioned from free DNA by capturing all the protein on paramagnetic beads. First, 25 μl of tosyl coupling buffer (0.5M Na₂HPO₄/0.12 M NaOH) was added to the protein-DNA mixture, raising the pH to ˜10. Then, 0.3 mg of M-280 tosyl-activated paramagnetic beads (Dynal) in 25 μl of 10 mM NaPO₄ (pH 6.5) were added, and the mixture was incubated for 5 minutes at 75° C. The excess tosyl sites were then blocked by addition of 25 μl of capping/blocking buffer (0.25M glycine/1% bovine serum albumin, adjusted to pH9 with NaOH) and incubated at 75° C. for an additional 5 minutes.

The beads were then washed 2 times in 100 μl 20 mM NaOH and 3 times in 100 μl of Protease Master Mix (10 mM Na₂HPO₄/2M urea/1% SDS), and resuspended in 95 μl of Protease Master Mix. These washes are intended to remove all DNA not covalently bound through protein to the tosyl beads.

Protein-DNA complexes were released from the beads, and the protein component digested, by the addition of 5 μl 20 mg/ml proteinase K, incubated at 60° C. for 10 minutes.

Before the DNA can be amplified by PCR, the proteinase K and PCR interferants such as SDS and urea must be removed. This is accomplished by primer-bead capture and washing. Dynal M270 beads coated with the sequence:

5′TTTTTTTTTCCCGCTCGTCGTCTG 3′ SEQ ID NO:74

which is complementary to the 3′ fixed region of the aptamer, are suspended in 5M NaCl at a concentration of 4 mg/ml. Then, 25 μl of this suspension was added to the protease digest solution, and the hybridization capture reaction was allowed to proceed for 15 minutes at 50° C. with occasional agitation. The bead suspension was washed 5 times with 100 μl 1×SB. The DNA was eluted from the beads by addition of 80 μl 20 mM NaOH. The DNA solution was then neutralized by the addition of 20 μl 80 mM HCl.

The aptamer solution was prepared for amplification by the addition of 25 μl of 5×SQ9 PCR Buffer+primer+radiolabel+Taq [200 mM Tris, pH 8.3, 186 mM KCl, 12.5 mM MgCl₂, 1 mM each dATP, dCTP, dGTP, and 5-BrdUTP, 5×SYBR I Green (e.g. a 1:2,000 dilution of manufacturer stock) 5 μM each (AT)₄-5P7 and (AB)₂-(T)₈-3P7.1/˜1 Ci³²P-(AT)₄-5P7), 1.25 U/μl AmpliTaq DNA polymerase]. PCR amplification was for 25 cycles at 96° C./15 seconds, 75° C./60 seconds. After purification on streptavidin beads and NaOH elution (see above), this procedure yielded an average of 26 pmol DNA, as measured by liquid scintillation. Target protein concentrations for the next round were chosen to maintain a signal of 2-fold over the no-protein control. That is, if a given round had a signal 10-fold that of the no-protein control, the target concentration was reduced 5-fold in the subsequent round.

All of the washes and eluates were recovered and counted by Cerenkov scintillation for 3 minutes, in order to track the recovery of the radiolabeled DNA pools from each SELEX round. These data allow one to track the efficiency of each step in the process: the fraction of captured DNA-protein complexes that are released by protease digestion; the fraction of digested complexes that are captured on primer beads; and the fraction of captured DNA that is eluted from primer beads. On average, each of these steps is a little better than 50% efficient, resulting in an overall recovery of ˜20% of the DNA that was initially captured on to the tosyl (Ts) beads. The efficiency of the tosyl bead capture was variable, dependent on the activity of the evolving aptamer pool, as well as its intrinsic efficiency, and so was not evaluated.

These data were also used to monitor the progress of the selection. Table 2 shows the fraction of radiolabeled DNA captured on to tosyl beads, which reflects the activity of the selected pools. Because protein concentrations were reduced to increase selection stringency (see Table 1), the pool activity from round to round is not directly reflected in the fraction bound.

TABLE 2
Fraction of SELEX pools captured on to tosyl beads.

| | Round 1 | Round 2 | Round 3 | Round 4 | Round 5 | Round 6 |
|---|---|---|---|---|---|---|
| hNE | 2.2E−02 | 1.3E−03 | 1.2E−03 | 4.7E−03 | 3.8E−03 | 4.7E−03 |
| gp120_{MN} | 1.2E−03 | 3.1E−03 | 5.6E−02 | 7.9E−03 | 2.0E−02 | 5.5E−03 |
| IgE | 4.6E−04 | 1.3E−03 | 2.7E−04 | 3.4E−03 | 9.3E−04 | 5.4E−03 |
| L−selectin | 3.3E−04 | 2.0E−03 | 7.0E−04 | 5.3E−04 | 3.5E−04 | 4.3E−03 |
| PDGF | 6.4E−03 | 2.7E−03 | 1.0E−03 | 2.4E−03 | 2.0E−03 | 6.9E−03 |
| Thrombin | 7.0E−04 | 1.5E−03 | 5.3E−04 | 8.3E−03 | 1.5E−02 | 1.3E−02 |
| bFGF | l.2E−03 | 3.1E−03 | 1.2E−02 | 4.7E−03 | 1.7E−03 | 6.2E−03 |
| no protein | 1.2E−03 | 3.6E−03 | 6.3E−04 | 1.6E−03 | n/a | 1.2E−02 |

After six rounds of selection, the aptamer libraries were tested for crosslinking activity to their respective targets. Trace amounts of radiolabeled DNA were mixed with target protein at a series of protein concentrations, irradiated with 308 nm light to 5 J/cm² to form DNA-protein conjugates. Then, 1M urea and 1 mM tricarboxyethylphosphine (TCEP) were added and the mixture heated to 95° C. for 1 minute to denature any non-covalent DNA-protein complexes. The remaining covalent complexes were trapped by vacuum filtration on 0.45 μm nitrocellulose filters. A portion of each sample was trapped on positively charged nylon filters (which bind both free and complexed DNA) to serve as a reference. The filters were counted and the fraction of cpm trapped on nitrocellulose filters (which is the fraction of nucleic acid that photocrosslinked to protein) was determined and plotted as a function of protein concentration in FIG. 9.

Five of the seven pools, including the spiked bFGF control experiment, show significant protein-dependent binding indicative of photocrosslinking nucleic acid ligand activity. The activity of these pools was confirmed by SDS-PAGE analysis of crosslinking at 0, 40, 100 nM target protein, with a control of 1 nM protein but no irradiation (N) as shown in FIG. 10. The DNA-protein conjugates enter the gel poorly and tend to stick to the well. Furthermore, some of the free DNA also sticks to the well. However, for the five SELEX pools that show activity in the filter-binding assay, all show a light-dependent, protein-dependent band indicating aptamer-DNA crosslinking, illustrated in FIG. 10.

A second series of experiments fixed the target protein concentration at 100 nM and varied the light dose. The putative crosslinking bands were generated in a light-dose dependent fashion, further confirming the photocrosslinking activity of the selected pools as illustrated in FIG. 11.

Active pools were cloned and sequenced by standard methods. The bFGF pool, which had been seeded with 10⁶ copies of the bFGF photocrosslinking nucleic acid ligand 0615, was found to consist predominantly of that sequence: 18/25 recovered. All 18 copies are perfect replicas of the parent sequence, demonstrating that the photoSELEX process is not highly mutagenic. Two of the 25 sequences are bFGF photocrosslinking nucleic acid ligand 0650, which was not deliberately introduced into the experiment and must have arisen and been selected as a laboratory contaminant. The remaining sequences are novel.

The gp120 pool also re-selected a laboratory contaminant, photocrosslinking nucleic acid ligand 0518, present as 4/34 of the sequenced aptamers. However, two other novel and unrelated sequences were also represented four times in the pool.

All other pools consisted entirely of novel aptamer sequences. These pools varied in their levels of “convergence”, that is, the degree to which one or a few sequences, or sequence motifs, comprise a large fraction of the pool. For instance, the elastase pool contained no repeat sequences, whereas 17/33 clones in the thrombin pool are the same sequence.

In order to confirm the ability of the solution photoSELEX process to select active photocrosslinking nucleic acid ligands, individual aptamers were prepared and tested for photocrosslinking to their target proteins as described in examples 9 and 10 below. Aptamer sequences were chosen to reflect different levels of representation, base composition and sequence motifs. These clones were characterized for protein- and light-dependent crosslinking by filter-binding and denaturing gel electrophoresis assays. In summary, all individual aptamers show crosslinking activity against their target proteins, with one exception: a sequence from the elastase pool which contains no 5-BrdU residues. Data for affinity, extent of crosslinking and crosslinking rate are shown below in Table 3; “K_{D}” is the apparent binding constant derived from a plot of target concentration vs fraction aptamer crosslinked; “X-link plateau” is the plateau value of this plot. “Rate” is the apparent first-order rate constant for crosslinking at a fixed target concentration (25 nM), with respect to total light dose. Note that the sequences referred to in Table 3 are provided in Table 7.

TABLE 3
photocrosslinking nucleic acid ligand characterization.

| | | | | | | SEQ. |
|---|---|---|---|---|---|---|
| | | | X−link K_{D} | X−link | Rate | ID. |
| | Pool | Clone | (nM) | Plateau (%) | (J⁻¹ cm² ) | NO. |
| hNE | 2 | 18 | 21 | 37 | 0.72 | 1 |
| | 2 | 43 | 61 | 27 | 0.14 | 2 |
| | 2 | 73 | 29 | 32 | 0.28 | 3 |
| gp120_{MN} | 3 | 5 | 5 | 68 | 0.19 | 4 |
| | 3 | 76 | 9 | 72 | 0.5 | 5 |
| PDGF | 4 | 24 | 2 | 52 | 0.25 | 6 |
| | 4 | 64 | 4 | 53 | 0.2 | 7 |
| | 4 | 87 | 2 | 36 | 0.29 | 8 |
| Thrombin | 5 | 4 | 0.03 | 25 | 0.4 | 9 |
| | 5 | 51 | 0.04 | 6 | 0.4 | 10 |
| | 5 | 75 | 110 | 62 | Nd | 11 |
| | 5 | 77 | 0.05 | 9 | 0.64 | 12 |

### Example 6

### Methods for Synthesizing Target Beads: Protein Biotinylation and Attachment to Streptavidin Paramagnetic Beads

A. Biotinylation of Proteins on Carbohydrates with Biotin-LC-hydrazide

Protein (0.4 nmol) was exchanged into 0.1 M NaOAc, pH 5.5, 0.01% Zwittergent 3/14 using a microcon with the appropriate MW cutoff filter for each protein. The buffer was spun out and replaced three times, and the protein concentrated to 100 μL. Sodium periodate (0.3 M in 0.1 M NaOAc, pH 5.5) was then added to the protein solution to give a final concentration of 20 mM sodium periodate. The solution was incubated in the dark at RT for 30 minutes. 50% glycerol was added to the solution to give a final concentration of 60 mM to terminate the reaction.

The sodium periodate was removed by passing the solution over a NAP-10 column equilibrated in 0.1 M NaOAc, pH 5.5. Ten-drop fractions were collected, and the A₂₆₀ values measured for each. The fractions with the highest absorbance values were pooled for each protein and transferred into opaque tubes. Biotin-LC-hydrazide (50 mM; Pierce cat#21340) in DMSO was added to each protein solution to give 5 mM biotin. The reaction was incubated for 1 hour at RT with rotating. The reaction was quenched with 100 μL of 1 M Tris-HCl, pH 7.5.

Excess biotin was removed by exchanging the buffer into PBS using a microcon with an appropriate MW cutoff filter. The buffer was spun out and replaced three times.

B. Biotinylation of Proteins Through Carboxyl Groups with Biotin-LC-hydrazide and EDC Activation

Protein (0.4 nmol) was exchanged into 0.1 M MES, pH 5, 0.01% Zwittergent 3/14 using a microcon with the appropriate MW cutoff filter for each protein. The buffer was spun out and replaced three times, and the protein concentrated to 100 μL. Biotin-LC-hydrazide (50 mM′ Pierce cat#21340) in DMSO was added to each protein solution to give 50:1 biotin:protein. The reaction was incubated for 1 hour at RT with rotating. Then, 520 mM EDC in 0.1 M MES, pH 5, was added to the protein/biotin solution to give 0.5 mM EDC. The reaction was incubated overnight at RT with rotating. Excess biotin was removed by exchanging the buffer into PBS using a microcon with an appropriate MW cutoff filter. The buffer was spun out and replaced three times.

C. Photobiotinylation of Proteins

First, 4 nmol or 200 μg, whichever was less, protein was exchanged into PBS, 0.01% Zwittergent 3/14 using a microcon with the appropriate MW cutoff filter for each protein. The buffer was spun out and replaced three times, and the protein concentrated to 100 μL. Then, 25 mg/ml photoactivatable biotin (Pierce cat#29987) in DMSO was added to each protein solution to give 50:1 biotin:protein. The reaction was placed into a microtiter plate well. The plate was placed 15 cm below a black light and irradiated for 15 minutes at 4 ° C.

Excess biotin was removed by exchanging the buffer into PBS using a microcon with an appropriate MW cutoff filter. The buffer was spun out and replaced three times.

D. Loading of Biotinylated Proteins Onto Streptavidin Beads

Dynal M280 streptavidin beads (2 mg) were washed three times with PBS using magnetic separation. The final wash solution was removed from the beads. The beads were resuspended in the biotinylated protein solution and mixed at RT for 30 minutes. The protein solutions were removed from the beads by magnetic separation, and the beads were resuspended in 1 mg/ml biotin in PBS to cap any unreacted streptavidin molecules. The beads were mixed again at RT for 15 minutes. The biotin solution was removed and the beads were washed three times with 5×SB. The beads were resuspended in 5×SB to give a 12 mg/ml solution, and used for the affinity SELEX process.

### Example 7

### The Automated Bead Affinity SELEX Process

The following example uses the automated apparatus described in Example 2; buffer compositions are as described above unless noted otherwise.

First the robot is preloaded with:

| | | |
|---|---|---|
| Bottled solutions: | 1. | 1X SB |
| | 2. | 5X SB |
| | 3. | 20 mM NaOH |
| | 4. | 80 mM HCl |
| | 5. | dH₂O |
| Consumables: | 1. | disposable tips |
| | 2. | 96-well reaction plates |
| Reagents: | 1. | target beads in at 12 mg/ml in 5X SB (50 μL per |
| | | reaction in targetRack) |
| | 2. | random DNA library, 1 uM in dH₂O (100 μL per |
| | | reaction in falcon15Rack) |
| | 3. | streptavidin beads (Pierce MagnaBind) at |
| | | 5 mg/mL in 5M NaCl (25 μL per reaction in |
| | | falcon7Rack) |

The following steps are then performed in order (all steps are done at room temperature unless otherwise noted; all steps done by robot unless otherwise noted):

A. Dispense DNA Library

1. Transfer 100 μL DNA library from falcon15Rack to selectionModule

B. Target Bead Dilution

1. Dispense 5×SB into dilutionRack

2. Shake targetRack 10 seconds to mix target beads

3. Transfer target beads from targetRack to dilutionRack to final bead concentration of 2.4 mg/mL

4. Shake dilutionRack 10 seconds to mix target beads

5. Transfer 25 μL (=300 μg) diluted target beads to selectionModule

C. Selection and Washes

1. Shake selectionModule 15 minutes to mix target beads and DNA and equilibrate

2. Wash target beads 5 times with 100 μL 1×SB

a) Insert magnets to draw beads to side of tube

b) Aspirate buffer to waste

c) Dispense 100 μL buffer into tube

d) Withdraw magnets

e) Shake selectionModule 30 seconds to mix beads

D. Elution and Neutralization

1. Insert magnets to draw beads to side of tube

2. Aspirate buffer to waste

3. Dispense 85 μL 20 mM NaOH

4. Withdraw magnets

5. Shake selectionModule 60 seconds to mix target beads and elute aptamer DNA

6. Insert magnets to draw beads to side of tube

7. Dispense 20 μL 80 mM HCl to pcrRack

8. Transfer 80 μL eluted DNA from selectionModule to pcrRack

E. Amplification

1. Manually load enzymeRack with Taq DNA Polymerase (1.25 μL per reaction)

2. Manually load falcon15Rack with 5×SQ9 PCR Buffer+primer (200 mM Tris-HCl, pH 8.3, 186 mM KCl, 12.5 mM MgCl₂, 1 mM each dATP, dCTP, dGTP, 5-BrdUTP, 5×SYBR Green 1, 5 uM primer (AT)₄-5P7, 5 um primer (AB)₂-(T)₈-3P7.1), 23.75 μL per reaction

3. Transfer Taq DNA Polymerase from enzymeRack to falcon15Rack

4. Mix by aspiration/dispense

5. Transfer 25 μL of this mixture from falcon15Rack to pcrRack

6. Manually seal reactions with optical caps and run quantitative PCR offline on ABI GeneAmp 5700 (20 cycles of 96° C. for 15 seconds, then 75° C. for 60 seconds)

7. Manually return pcrRack to robot and remove optical caps

F. Purification

1. Shake falcon7Rack 15 seconds to mix streptavidin beads

2. Transfer 25 μL streptavidin beads from falcon7Rack to purificationModule

3. Transfer 100 μL amplification product from pcrRack to purificationModule

4. Shake purificationModule 5 minutes to mix streptavidin beads and equilibrate

5. Wash streptavidin beads 3 times with 100 μL 1×SB (as above)

6. Elute aptamer strand with 20 mM NaOH (as above)

7. Dispense 20 μL 80 mM HCl in dnaArchiveRack

8. Transfer 80 μL eluted aptamer from purificationRack to dnaArchiveRack

9. Manually load new 96-well plate in selectionModule

10. Transfer 50 μL neutralized aptamer from dnaArchiveRack to selectionModule

11. Dispense 50 μL dH₂O to selectionModule

This constitutes round 1 of the automated affinity SELEX process. Subsequent rounds use the neutralized aptamer solution dispensed to the selectionModule in step F.10 as candidate mixture rather than the DNA library stored in the falcon15Rack (therefore, step A.1 is not performed after round 1). The DNA concentration in subsequent rounds was 100-200 nM. The concentration of target beads in step B.3, and hence the quantity of target beads dispensed to the selectionModule in step B.5, was lowered in response to a high selection signal. Selection signals during quantitative PCR ranged from le7-le11 copies DNA, limited on the lower end by protein-independent retention of DNA by the bead surface and selection vessel surface. Protein concentrations were lowered 10-fold when selection signal exceeded le10 copies.

### Example 8

### The Automated Solution PhotoSELEX Process

The following example uses the apparatus described in example 2 above; buffer compositions are as described above unless noted otherwise.

First, the Robot is pre-loaded with:

| | | |
|---|---|---|
| Bottled solutions: | 1. | 1X SSB |
| | 2. | 1X SB |
| | 3. | 1X Guanidinium Wash Buffer (1X GWB) (4M |
| | | guanidinium thiocyanate, 2% Sakosyl, 2 mM |
| | | EDTA, 2 mM TCEP, 25 mM HEPES, pH 7.5) |
| | 4. | 20 mM NaOH |
| | 5. | 80 mM NaOH/0.025% TWEEN |
| | 6. | 80 mM HCl |
| | 7. | dH₂O |
| Consumables: | 1. | disposable tips |
| | 2. | 96-well reaction plates |
| Reagents: | 1. | target protein in 1X SSB at various concentrations |
| | | (50 μL per reaction in targetRack) |
| | 2. | random DNA library, 1 uM in 1X SSB (100 μL |
| | | per reaction in falcon15Rack) |
| | 3. | tosyl beads, 12 mg/mL in 5 mM sodium |
| | | phosphate, pH 6.5 (25 μL (= 300 ug) per reaction |
| | | in falcon7Rack) |
| | 4. | tosyl coupling buffer (0.5M Na₂HPO₄, 0.12M |
| | | NaOH) (25 μL per reaction in falcon15Rack) |
| | 5. | capping/blocking buffer (0.25M glycine/1% |
| | | bovine serum albumin, adjusted to pH 9 with |
| | | NaOH) (25 μL per reaction in falcon15Rack) |
| | 6. | primer beads (Dynal M270 coated with sequence |
| | | complementary to 3′ fixed sequence region of |
| | | candidate mixture) at 4 mg/mL in 5M NaCl |
| | | (25 μL per reaction in falcon7Rack) |
| | 7. | streptavidin beads (Pierce MagnaBind) at |
| | | 5 mg/mL in 5M NaCl (25 μL per reaction in |
| | | falcon7Rack) |

The following steps are then performed in order (all steps are done at room temperature unless otherwise noted; all steps done by robot unless otherwise noted):

A. Dispense DNA Library

1. Transfer 100 μL DNA library from falcon15Rack to selectionModule

B. Target Protein Dilution

1. Dispense 133 SSB into dilutionRack

2. Transfer target protein from targetRack to dilutionRack

3. Transfer 25 μL diluted target protein to selectionModule

C. Photo-selection

1. Wait 15 minutes to equilibrate

2. Irradiate with laser tool

D. Protein Capture and Denaturing Washes

1. Transfer 25 μL of tosyl coupling buffer from falcon15Rack to selectionModule

2. Shake falcon7Rack 30 seconds to mix tosyl beads

3. Transfer 25 μL tosyl beads from falcon7Rack to selectionModule

4. Shake selectionModule 30 seconds to mix tosyl beads

5. Manually transfer selection plate to MJ Research PTC-200 and incubate 75° C., 5 minutes

6. Manually return selection plate to selectionModule

7. Transfer 25 μL capping/blocking buffer from falcon15Rack to selectionModule

8. Shake selectionModule 15 seconds to mix tosyl beads

9. Manually transfer selection plate to MJ Research PTC-200 and incubate 75° C., 2 minutes

10. Manually return selection plate to selectionModule

11. Wash tosyl beads 2 times with 100 μL 20 mM NaOH/ 0.025% TWEEN

a) Insert magnets to draw beads to side of tube

b) Aspirate buffer to waste

c) Dispense 100 μL wash buffer

d) Withdraw magnets

e) Shake selectionModule 30 seconds to mix beads

12. Wash tosyl beads 3 times with 100 μL 1×GWB (as above)

E. Protease Digestion

1. Manually load Proteinase K at 20 mg/mL in enzymeRack (5 μL per reaction)

2. Manually load Protease Master Mix (10 mM Na₂HPO₄/2M urea/1% SDS) in falcon7Rack (95 μL per reaction)

3. Transfer Proteinase K from enzymeRack to falcon7Rack

4. Mix by aspiration/dispense

5. Transfer 100 μL Protease Master Mix+Proteinase K from falcon7Rack to selectionModule and resuspend tosyl beads

6. Shake selectionModule 30 seconds to mix tosyl beads

7. Manually transfer selection plate to MJ Research PTC-200 and incubate 65° C., 10 minutes

8. Manually return selection plate to selectionModule

F. Antamer Capture and Wash

1. Shake falcon7Rack 15 seconds to mix primer beads

2. Transfer 25 μL primer beads from falcon7Rack to selectionModule

3. Shake selectionModule 15 seconds to mix primer beads

4. Manually transfer selection plate to MJ Research PTC-200 and incubate 50° C., 15 minutes

5. Manually return selection plate to selectionModule

6. Wash primer beads 5 times with 100 μL 1×SB (as above)

G. Elution and Neutralization

1. Insert magnets to draw beads to side of tube

2. Aspirate buffer to waste

3. Dispense 85 μL 20 mM NaOH

4. Withdraw magnets

5. Shake 60 seconds to mix and elute aptamer DNA

6. Insert magnets to draw beads to side of tube

7. Dispense 20 μL 80 mM HCl to pcrRack

8. Transfer 80 μL eluted DNA from selectionModule to pcrRack

H. Amplification

1. Manually load enzymeRack with Taq DNA Polymerase (1.25 μL per reaction)

2. Manually load falcon15Rack with 5×SQ9 PCR Buffer+primer (200 mM Tris-HCl,

pH 8.3, 186 mM KCl, 12.5 mM MgCl₂, 1 mM each dATP, dCTP, dGTP, 5-BrdUTP,

5×SYBR Green 1, 5 μM primer (AT)₄-5P7, 5 μM primer (AB)₂-(T)₈-3P7.1) (23.75 μL per reaction)

3. Transfer Taq DNA Polymerase from enzymeRack to falcon15Rack

4. Mix by aspiration/dispense

5. Transfer 25 μL of this mixture from falcon15Rack to pcrRack

6. Manually seal reactions with optical caps and run quantitative PCR offline on ABI GeneAmp 5700 (20 cycles of 96° C. for 15 seconds, then 75° C. for 60 seconds)

7. Manually return pcrRack to robot and remove optical caps

I. Purification

1. Shake falcon7Rack 15 seconds to mix streptavidin beads

2. Transfer 25 μL streptavidin beads from falcon7Rack to purificationModule

3. Transfer 100 μL amplification product from pcrRack to purificationModule

4. Shake purificationModule 5 minutes to mix streptavidin beads and equilibrate

5. Wash streptavidin beads 3 times with 100 μL 1×SB (as above)

6. Elute aptamer strand with 20 mM NaOH (as above)

7. Dispense 20 μL 80 mM HCl in dnaArchiveRack

8. Transfer 80 μL eluted aptamer from purificationRack to dnaArchiveRack

9. Manually load new 96-well plate in selectionModule

10. Transfer 50 μL neutralized aptamer from dnaArchiveRack to selectionModule

11. Dispense 50 μL dH₂O to selectionModule This constitutes the first round of the automated solution photoSELEX process. Subsequent rounds use the neutralized aptamer solution dispensed to the selectionModule in step I.10 as candidate mixture rather than the DNA library stored in the falcon15Rack (therefore, step A.1 is not performed after round 1). Target protein concentrations for the next round were chosen to maintain a signal of 2-fold over the no-protein control. That is, if a given round had a signal 10-fold that of the no-protein control, the target concentration was reduced 5-fold in the subsequent round.

### Example 9

### Affinity Assays

Aptamer DNA is radiolabeled to a specific activity 2×10⁵ cpm/pmol (see Example 11) and heated at 75° for 2-3 minutes to break up any aggregates that may have formed. Target protein and aptamer DNA, both in 1×FSB (see example 4), are mixed in the wells of a 96-well plate to give a protein dilution series in which the final aptamer DNA concentration is held constant at 100 pM and the target protein concentration varied to form a dilution series of 100, 33, 11, 3.7, 1.2, 0.41, or 0.14 nM. A no protein control is also included. The target protein dilution series occupies one column of the 96-well plate. Suitable plates include Sigma polypropylene half-area, Cat. No. P-2856, Costar vinyl assay plates, Cat. No. 2596, or Costar thermowell plate, Cat. No. 6509. The target protein and aptamer DNA mixtures are then equilibrated at room temperature for 5 minutes.

The target protein and aptamer DNA mixtures are then vacuum filtered on a nitrocellulose filter. DNA that has bound to protein is retained on the surface of the filter, whereas unbound DNA passes through. Vacuum filtration can take place on a 12-well manifold (Millipore), or on a 96-well manifold (Gibco Cat. No. 11055-019).

For the 12-well manifold, a 25 mm nitrocellulose filter disk (Millipore HAWP02500) is placed on each well of the manifold. Then, 1 ml of 1XFSB is pipetted into each manifold well, and each is inspected for drainage that would indicate a leak in the seal around each filter. Unused wells are plugged, and a vacuum is applied to the manifold to check for rapid drainage, confirming that the filters are not clogged or blocked. With the vacuum on, target protein-aptamer DNA mixtures from the 96-well plate are pipetted into the manifold wells. Immediately after adding a target protein-aptamer DNA mixture to a manifold well, that well is washed with 1 ml of 1×FSB before pipetting the next target protein-aptamer DNA mixture into the next free manifold well. Following vacuum filtration, each filter is removed from the manifold and placed in a 7 ml scintillation tube. Fifty μl of remaining aptamer DNA mix is pipetted into a final scintillation tube as a 100% reference control. The tubes are counted for 1 minute, 5% 2σ level.

For the 96-well manifold, nitrocellulose membrane (Life Technologies Cat. No. 1146040) is pre-wet in 1×FSB minus HSA and placed on the manifold. Using a multi-channel pipettor, each well of the membrane is wetted with 100 μL 1×FSB and checked to see that draining is rapid and uniform. Forty μL of each target protein-aptamer DNA mixture is added to the manifold wells and immediately followed by 60 μL 1×FSB as a rinse. Reference control samples are made by filtering 10 μl from each DNA protein mixture on to a positively charged nylon membrane (Millipore Immobilon-Ny+ Cat. No. INYC09120), which traps 100% of the DNA. The reference wells are immediately washed with 60 μL 1×FSB. The nitrocellulose and nylon membranes are placed on a solid support, covered with Saran wrap and exposed to a phosphorimager screen for 0.5-2 hrs.

In either case, the fraction of aptamer DNA bound to target protein is determined by counting the radioactivity on the nitrocellulose filter as compared to the 100% reference control sample, and subtracting the background radioactivity of the no target protein control.

### Example 10

### PhotoCrosslink Assays

Assays are set up in a 96-well plate (Hybaid 96×0.3 ml, HB-TC-4072N) as for the affinity assay in Example 9, except that target protein-aptamer DNA mixture volumes are 75 μL in 1×FSB. The final DNA concentration cannot exceed the lowest concentration of protein, and should be at least 2-fold less. Generally speaking this will be a final DNA concentration of 200 pM or less. A second 100 nM target protein dilution is included as a no irradiation control and replaces the 0.14 nM protein sample. After the protein samples and DNA are equilibrated (>5 minutes at room temperature), all wells (except the no irradiation control) are irradiated with 308 mn light at a dose of 5 J/cm^{2 .} Following sample irradiation, protein aggregation is prevented by the addition to the irradiated mixtures of 4 μL of 100 mM tri(2-carboxyethyl) phosphine (TCEP) and 53 μL of 5 M urea to final concentrations of 5 mM and 2 M respectively in a volume of 132 μL.

Immediately prior to loading the target protein-aptamer DNA mixtures on the nitrocellulose and the nylon (100% reference control) membranes, the mixtures are heated to 95° C. for 3 minutes to denature protein. This allows one to distinguish between covalent and non-covalent complexes. Sixty μL of each target protein-aptamer DNA mixture is filtered on nitrocellulose as described in example 9 using the 96-well manifold, except that 1×FSB minus HSA is used both to pre-wet the membranes and for all washes. Eighteen μL of each DNA protein mixture is filtered onto a nylon membrane as described above to serve as a 100% reference control. The membranes are then exposed and the fraction of crosslinked DNA is calculated as described in example 9.

In addition, 10 μL each of the 100 nM target protein-aptamer DNA mixtures (+/−irradiation) and the no protein control are run on a 10% polyacrylamide TBE-urea gel. Prior to loading on the gel, the three samples are mixed with 5 μL formamide loading buffer (0.1×TBE, 0.1% SDS, 1 mM EDTA, 0.02% xylene cyanol, 0.02% bromophenol blue, 50% formamide) and heated to 75° C. for 3-5 minutes. The gel is run at 35 W until the bromophenol blue dye front is close to the bottom of the gel, and then imaged on a phosphorimager for between 30 minutes and 2 hours. Free (uncrosslinked) aptamer runs at or slightly below the xylene cyanol marker, whereas crosslinked product runs above.

### Example 11

### Method For Radioactively Labeling of Aptamers for Use in Activity Assays

In examples 9 and 10 above, aptamer solutions are radioactively labeled in order to determine the fraction of aptamer that remains on a nitrocellulose or nylon membrane. The following is a method for radioactively labeling and purifying single-stranded aptamer DNA using a 96 well plate format where the expected input DNA concentration into the labeling reaction is about 100 nM.

For DNA with a 5′OH or inverted 3′ end, a T4 labeling master mix comprising per 8 reactions 15 μl 10×PNK buffer (NEB or Gibco), 15 μl water, 3.0 μl γ32P-ATP, 3000 Ci/mmol, 10 mCi/ml (NEN) and 1.0 μl polynucleotide kinase (NEB or Gibco) is made up. For DNA with 5′ modifications, a terminal transferase labeling master mix comprising per 8 reactions: 15 μl 10×NEB Buffer 4 (NEB), 15 μl 2.5 mM CoCl2 (NEB), 3 μl a32P-ATP, 3000 Ci/mmol, 10 mCi/ml (NEN) and 1 μl terminal transferase (NEB) is made up. Four μl of the appropriate labeling mix per reaction is then distributed to each well of one column of a 96-well plate (Millipore multiscreen plate #MAHVN4510, Costar vinyl assay plate #2596, Costar thermowell plate #6509, or M J Research multiplate #MLL-9601). From this column, a multichannel pipetter is used to distribute 3.51 μl per reaction to the appropriate wells in the plate. Then, 11 μl of the DNA aptamer preparation is added to each well (DNA can be 2 pmol synthetic aptamer, enzymatically-prepared clones, or enriched or random SELEX libraries). The plate is sealed with mylar or foil tape to prevent evaporative loss and incubated 37° C./30 min. Then, 10.5 μl TE is added, and the reactions are heat-killed at 65° C./5 min.

Removal of Unincorporated Label

Depending on the number of samples being processed, individual G-50 columns (Amersham Pharmacia Biotech cat #27-5330-02) may be used for sample cleanup. An alternative for larger numbers of samples is the 96-well SEQueaky Kleen Dye Terminator removal kit from BioRad, cat#732-6260). In either case, clean-up is performed according to the instructions supplied with these kits.

TLC Assay

A 20×20 cm PEI-cellulose plastic-backed TLC plate (JT Baker #4473-04 or Sigma #801063) is cut to 20×8 cm. Then, 0.5 μl of each kinase reaction is spotted 1.5 cm from the bottom of the plate. For single-species samples whose concentration is known prior to labeling, samples may be spotted both before and after removal of unincorporated label to determine labeled aptamer concentration and specific activity. The plate is air-dried for 5 minutes, then developed by chromatography in 0.75M KH₂PO₄. When the solvent front is 0-1 cm from the top of the plate, the plate is removed, wrapped in saran wrap, and exposed on a phosphorimager plate for 10-30 minutes. Polynucleotides are retained at the origin, whereas ATP and phosphate run higher. At least 85% of the counts should be in the polynucleotide.

Scintillation Counting

0.5 μL of post-G50 cleaned-up sample is placed in a scintillation vial containing approximately 2 mL of scintillation fluid. Alternatively, the pipette tip containing the radiolabeled sample may be directly ejected into an empty scintillation vial. If there is enough sample, duplicates should be read on the scintillation counter. The rack of samples is placed in the scintillation counter and readings are taken.

### Example 12

### PCR Amplification and Cloning of Enriched Candidate Mixtures (Pools)

Pools of nucleic acid ligands produced by the automated SELEX process are PCR amplified, cloned, and sequenced in order to further characterize the nucleic acid ligands contained therein. The PCR amplification of pools must be performed under conditions that preserve the sequence diversity of each pool, while at the same time producing ample product for cloning.

30N7.1 and 40N7.1 5-BrdU pools (1:10,000 dilution of the pool; pool concentration is typically 0.1-1 μM) are amplified in SQ10 PCR Buffer [40 mM Tris-HCl, pH 8.3, 50 mM KCl, 2.5 mM MgCl₂, 0.2 mM dATP/dCTP/dGTP/dTTP, 1×SYBR Green], containing 100 pmol each of (AT)₄-5P7 and (T)₈-3P7.1, 5 U of AmpliTaq. The volume of the PCR reaction is 100 μL. The reactions are then cycled on a GeneAmp 5700 thermal cycler and two step PCR is performed with 9 cycles of 96° C. for 15 seconds, 75° C. for 1 minute. These conditions limit formation of primer dimers and high molecular weight parasites.

One μL of each reaction is run on a native 8% polyacrylamide gel with 20 and 100 bp ladders, and also with 1 ng, 2 ng, and 5 ng of BioRad Amplisize Molecular Ruler (Cat. No. 170-8200) to assess PCR products and approximate quantity. When the randomized region (N) of the template is 30 bases in length, then the correct product is 77 bp; for N=40, the correct product length is 87 bp. The PCR product yield is approximately 1-1.5 ng/μL.

Prior to cloning the PCR products, a Qiagen MinElute PCR Purification Spin Column (Cat. No. 28006) is used to concentrate the dsDNA product, and remove primers, nucleotides, polymerase and salts. The products are eluted into a volume of 10 μL. One μL of each product is run on an 8% native 1×TBE acrylamide gel along with 20 and 100 bp ladders and BioRad AmpliSize Molecular 50-2000 bp Ruler in order to measure approximately the quantity of product. The usual product concentration after spin column purification is approximately 3-5 ng/μL.

The concentrated dsDNA PCR product is then cloned into the TOPO™ TA Cloning Kit (the pCR II-TOPO vector) using a 5 times molar excess of PCR product to vector according to the protocol supplied with the kit. The TOPO™ TA Cloning Kit uses topoisomerase instead of ligase. Topoisomerase recognizes and covalently binds to the 3′ thymidine on the pentameric sequence 5′-(C/T)CCTT-3′ at the 3′ phosphate, cleaves one strand of the DNA, allowing the DNA to unwind, and then re-ligates the ends. The reaction is done in 5 minutes, although improved efficiencies are sometimes seen with longer incubation times. Cloning efficiencies using this kit are>98%.

The ligated product is then transformed into bacteria according to the kit protocol. The transformed bacteria are plated onto LB plates (100 μg/ml Amp, 60 μg/ml X-Gal, 0.1 mM IPTG; TEKnova Cat. No. 0133-A100×), and incubated approximately 16 hours at 37° C. White colonies are then picked from the plates, and each used to inoculate 500 μL of 2-YT containing 100 μg/ml Amp in the wells of a 96-well plate. The plates are incubated at 280 rpm, 37° C. for 18 hours. Finally, 75 μL of each grown culture is transferred to a new well on a 96-well −80° C. plate and mixed with 75 82 L of 70% glycerol. The plates are then stored at −80° C. Plasmid inserts are sequenced by standard protocols.

### Example 13

### Automated Solution PhotoSELEX Process Experiment 1

The following table presents data obtained from an experiment performed in a 96-well format in which six rounds of the automated solution photoSELEX process were performed according to example 8. The initial candidate mixture for each automated solution photoSELEX process was 30N7.1 or 40N7.1 candidate mixture 5-BrdU DNA that had been ligand-enriched. For 30N7.1 DNA, the ligand-enrichment scheme comprised 1 round of the filter affinity SELEX (denoted herein by “1Fil”) according to example 4 above, followed by 5 rounds of the manual solution photoSELEX process (denoted herein by “5mSP”) performed according to example 5 above. For 40N7.1, the ligand-enrichment scheme comprised 5 rounds of the manual filter affinity SELEX process (denoted herein by “5Fil”).

For each target protein, a pool of photocrosslinking nucleic acid ligands was cloned and sequenced according to example 12 above. Binding data for each clone is displayed below in Table 4. “K_{D}” is the apparent binding constant derived from a plot of target concentration vs fraction aptamer crosslinked; “X-link plateau” is the plateau value of this plot. “Rate” is the apparent first-order rate constant for crosslinking at a fixed target concentration (25 nM), with respect to total light dose. The targets are HIV-1_{MN} gp120, Platelet Derived Growth Factor (PDGF), Angiogenin, Interleukin-4, β-Nerve Growth Factor (β-NGF), P-Selectin, and Transforming Growth Factor β1 (TGF-β1). The sequences are shown in Table 7.

TABLE 4

| | Candidate | | | X−link K_{D} | X−link | Rate | SEQ. ID. |
|---|---|---|---|---|---|---|---|
| | Mixture | Pool | Clone | (nM) | Plateau (%) | (J⁻¹ cm² ) | NO. |
| Gp120_{MN} | 30N7.1 | 007 | 3 | 3.8 | 46 | 0.7 | 13 |
| | 1Fil/5mSP | | | | | | |
| | | | 4 | 7.3 | 66 | 0.4 | 14 |
| | | | 11 | 8.0 | 30 | 0.14 | 15 |
| | | | 20 | 3.7 | 30 | 0.14 | 16 |
| PDGF | 30N7.1 | 008 | 26 | 1.8 | 67 | 0.43 | 17 |
| | 1Fil/5mSP | | | | | | |
| | | | 27 | 3.2 | 100 | 0.34 | 18 |
| | | | 31 | 5.3 | 100 | 0.19 | 19 |
| | | | 33 | 5.7 | 100 | 0.24 | 20 |
| | | | 35 | 5.1 | 71 | 0.07 | 21 |
| | | | 37 | 2.4 | 74 | 0.21 | 22 |
| Angiogenin | 40N7.1 | 011 | 11 | 1.1 | 23 | 1.46 | 23 |
| | 5Fil | | | | | | |
| | | | 12 | 0.1 | 31 | 0.68 | 24 |
| | | | 14 | 0.2 | 34 | 1.3 | 25 |
| | | | 16 | 0.3 | 28 | 2.6 | 26 |
| | | | 27 | 0.3 | 37 | 0.62 | 27 |
| | | | 58 | 2.2 | 16 | 1.58 | 28 |
| | | | 59 | 0.22 | 26 | 0.52 | 29 |
| | | | 85 | 0.2 | 31 | 3.5 | 30 |
| IL−4 | 40N7.1 | 012 | 8 | 39 | 40 | 1.8 | 31 |
| | 5Fil | | | | | | |
| | | | 31 | 21 | 49 | 0.98 | 32 |
| | | | 41 | 61 | 58 | 0.54 | 33 |
| | | | 48 | 4.3 | 52 | 0.17 | 34 |
| | | | 63 | ?? | | 0.71 | 35 |
| | | | 78 | 9.7 | 10 | 1.7 | 36 |
| B−NGF | 40N7.1 | 031 | 7 | 2.5 | 55 | 0.42 | 37 |
| | 5Fil | | | | | | |
| | | | 17 | 0.21 | 66 | 1.2 | 38 |
| | | | 43 | 7.9 | 38 | Nd | 39 |
| | | | 44 | 2.2 | 58 | 0.59 | 40 |
| | | | 65 | 0.2 | 47 | 0.94 | 41 |
| | | | 78 | 31 | 94 | 0.38 | 42 |
| P−selectin | 40N7.1 | 014 | 14 | 0.35 | 15 | 4 | 43 |
| | 5Fil | | | | | | |
| | | | 17 | 18 | 59 | 1.36 | 44 |
| | | | 21 | 9.1 | 31 | 0.76 | 45 |
| | | | 24 | 2.1 | 30 | 1.86 | 46 |
| | | | 95 | 45 | 39 | 1.54 | 47 |
| TGF−β1 | 40N7.1 | 015 | 74 | 4.9 | 79 | 0.34 | 48 |
| | 5Fil | | | | | | |
| | | | 81 | 7.2 | 80 | 0.58 | 49 |
| | | | 82 | 7.1 | 100 | 0.54 | 50 |
| | | | 83 | 4.3 | 80 | 0.82 | 51 |
| | | | 87 | 62 | 80 | 0.5 | 52 |

### Example 14

### Automated PhotoSELEX Process Experiment 2

The following table presents data obtained from an experiment performed in a 96-well format in which six rounds of the automated solution photoSELEX process were performed according to example 8. The initial candidate mixture for each automated solution photoSELEX process was 30N7.1 or 40N7.1 candidate mixture 5-BrdU DNA that had been ligand-enriched. For 40N7.1 DNA, the ligand-enrichment scheme comprised 5 rounds of the manual affinity SELEX process using nitrocellulose filter binding (denoted herein by “5Fil”) according to example 4 above. For 30N7.1, the ligand-enrichment scheme comprised 3 rounds of the automated bead affinity SELEX process using streptavidin paramagnetic beads and biotinylated target proteins (denoted herein by “3aBx,” wherein x designates the chemistry used to biotinylate the protein target) according to example 7. Target protein was biotinylated either through carboxyl groups (x=c), carbohydrate groups (x=s), or by using a photobiotinylation protocol (x=p) according to example 6 above.

For each target protein, a pool of photocrosslinking nucleic acid ligands was cloned and sequenced according to example 12 above. Binding data for each clone is displayed below in Table 5. “K_{D}” is the apparent binding constant derived from a plot of target concentration vs fraction aptamer crosslinked; “X-link plateau” is the plateau value of this plot. “Rate” is the apparent first-order rate constant for crosslinking at a fixed target concentration (25 nM), with respect to total light dose. The sequences are provided in Table 7.

TABLE 5

| | Candidate | | | X−link K_{D} | X−link | Rate | SEQ. ID. |
|---|---|---|---|---|---|---|---|
| | Mixture | Pool | Clone | (nM) | Plateau (%) | (J⁻¹ cm² ) | NO. |
| Interleukin−7 | 40N7.1 5Fil | 042 | 5 | 11 | 50 | Nd | 53 |
| Kininogen | 30N7.1 3aBc | 046 | 31 | 7.0 | 64 | Nd | 54 |
| L−Selectin | 30N7.1 3aBc | 048 | 21 | 7.0 | 25 | Nd | 55 |
| Plasmin | 40N7.1 5Fil | 050 | 25 | 78 | 50 | Nd | 56 |
| Serum Amyloid P | 40N7.1 5Fil | 051 | 50 | 0.54 | 55 | Nd | 57 |
| Thrombopoietin | 40N7.1 5Fil | 059 | 34 | 64 | 50 | Nd | 58 |

### Example 15

### Automated Solution PhotoSELEX Process Experiment 3

Seven rounds of the automated solution photoSELEX process were performed using either synthetic 30N7.1 5-BrdU DNA (obtained from Integrated DNA Technologies, Inc.), or 30N7.1 5-BrdU DNA (produced according to example 3) that was subjected to three rounds of the automated bead affinity SELEX process using streptavidin paramagnetic beads and biotinylated target proteins according to example 7 (denoted herein by “3aBx,” wherein x designates the chemistry used to biotinylate the protein target). Target protein was biotinylated either through carboxyl groups (x=c), carbohydrate groups (x=s), or by using a photobiotinylation protocol (x=p) according to example 6 above. Prior to beginning the automated solution photoSELEX process, the individual pools from the automated bead affinity SELEX process for each protein were combined. For example, for the target Coagulation Factor IX, three separate enriched pools were initially obtained by performing in separate wells of a 96-well plate three rounds of the automated bead affinity SELEX process with carbohydrate-biotinylated protein, photobiotinylated protein, and carboxyl-biotinylated protein respectively. These three separate pools were combined, and the combined pool (designated “30N7.1 3aBs,p,c” in the following table) was used to initiate seven rounds of the automated solution photoSELEX process. Binding data for each clone is displayed below in Table 6. “K_{D}” is the apparent binding constant derived from a plot of target concentration vs fraction aptamer crosslinked; “X-link plateau” is the plateau value of this plot. “Rate” is the apparent first-order rate constant for crosslinking at a fixed target concentration (25 nM), with respect to total light dose.

TABLE 6

| | | | | | X−link | | |
|---|---|---|---|---|---|---|---|
| | Candidate | | | X−link K_{D} | Plateau | Rate | SEQ. |
| | Mixture | Pool | Clone | (nM) | (%) | (J⁻¹ cm² ) | ID. NO. |
| Coagulation Factor IX | 30N7.1 3aBs,p,c | 87 | 50 | 6.2 | 75 | Nd | 59 |
| Coagulation Factor XII | 30N7.1 3aBs,p,c | 89 | 51 | 53 | 45 | Nd | 60 |
| Endostatin | 30N7.1 3aBp,c | 92 | 4 | 2.7 | 75 | Nd | 61 |
| Factor H | 30N7.1 3aBs,p,c | 94 | 12 | 14 | 65 | Nd | 62 |
| Collagen | 30N7.1 | 100 | 36 | 0.75 | 25 | Nd | 63 |
| Cytotoxic T lymphocyte− | 30N7.1 | 101 | 60 | 8.0 | 7 | Nd | 64 |
| associated protein−4 | | | | | | | |
| (CTLA−4) Fc | | | | | | | |
| Hepatocyte Growth | 30N7.1 | 107 | 23 | 0.53 | 73 | Nd | 65 |
| Factor HGF | | | | | | | |
| Insulin−like growth | 30N7.1 | 112 | 65 | 6.0 | 52 | Nd | 66 |
| factor binding protein−3 | | | | | | | |
| (IGFBP−3) | | | | | | | |
| UDP−glucuronosyl | 30N7.1 | 319 | 73 | 22 | 70 | Nd | 67 |
| transferase (UGT) 1A1 | | | | | | | |
| UGT 1A10 | 30N7.1 | 140 | 45 | 2.1 | 72 | Nd | 68 |
| UGT 1A3 | 30N7.1 | 141 | 53 | 3.9 | 70 | Nd | 69 |

### Example 16

Table 7 below lists the sequences of the photocrosslinking nucleic acids SEQ ID NO:1-69. Note that all the sequences include the tail sequences (AT)₄ and (A)₈ added to prevent the formation of high molecular weight parasites of the amplification procedure. It is to be understood that these sequences are not necessary for the function of the photocrosslinking nucleic acid ligands and may be deleted. Hence, photocrosslinking nucleic acid ligands with sequences substantially homologous to photocrosslinking nucleic acid ligands in Table 7 or with substantially the same structure as photocrosslinking nucleic acid ligands in Table 7 include photocrosslinking nucleic acid ligands lacking the 5′ (AT)₄ sequences and/or the 3′ (A)₈ sequence.

TABLE 7

| SEQ. | | |
|---|---|---|
| ID. | Protein | |
| NO. | Target | Sequence (5′→3′) |
| 1 | hNE | ATATATATGGGAGGACGATGCGGGCACATCACTCTATCATTTGCTACGGTACCGGAGTGAGTCCAGACGACGAGCGGGAAAAAAAA |
| 2 | hNE | ATATATATGGGAGGACGATGCGGCAACCCACCACTCTATCTTTCCCATAACTGCAGACGACGAGCGGGAAAAAAAA |
| 3 | hNE | ATATATATGGGAGGACGATGCGGGCCAATCTGTCTTCTTTCCATCCTTATGATCAGACGACGAGCGGGAAAAAAAA |
| 4 | gp120 | ATATATATGGGAGGACGATGCGGCAACCACACGCAGGAGGACACAACGATCCGCAGACGACGAGCGGGAAAAAAAA |
| 5 | gp120 | ATATATATGGGAGGACGATGCGGGACGAGGGACCAGACCGCCACAGCGGGATGCAGACGACGAGCGGGAAAAAAAA |
| 6 | PDGF | ATATATATGGGAGGACGATGCGGGCGGAAGAGGCAGGGTACCACGGCAGAGGTCAGACGACGAGCGGGAAAAAAAA |
| 7 | PDGF | ATATATATGGGAGGACGATGCGGGCGAAGGCACACCGAGTTCATAGTATCCCACAGACGACGAGCGGGAAAAAAAA |
| 8 | PDGF | ATATATATGGGAGGACGATGCGGGCCAACCCCTAGTGAACAACAACACTCCCACAGACGACGAGCGGGAAAAAAAA |
| 9 | Thrombin | ATATATATGGGAGGACGATGCGGGCAGTAGGTTGGGTAGGGTGGTCTGCTCAGACGACGAGCGGGAAAAAAAA |
| 10 | Thrombin | ATATATATGGGAGGACGATGCGGGAGGAGCTGATGGGTGGTGAGGTTGGCCAGACGACGAGCGGGAAAAAAAA |
| 11 | Thrombin | ATATATATGGGAGGACGATGCGGGCAGGACGGACAGCAAGGGGTGAGCACGAGCAGACGACGAGCGGGAAAAAAAA |
| 12 | Thrombin | ATATATATGGGAGGACGATGCGGGCGGTTGCTGTGGTTGGAAATGTCCCGTCAGACGACGAGCGGGAAAAAAAA |
| 13 | gp120 | ATATATATGGGAGGACGATGCGGGAGGACCACGACCATGACCCACCAGGAATGCAGACGACGAGCGGGAAAAAAAA |
| 14 | gp120 | ATATATATGGGAGGACGATGCGGGCACAGGCCTAACATACCTCCATCTCCTGGCAGACGACGAGCGGGAAAAAAAA |
| 15 | gp120 | ATATATATGGGAGGACGATGCGGGACCAACGAGACCACACGACAAGCGCTGTGCAGACGACGAGCGGGAAAAAAAA |
| 16 | gp120 | ATATATATGGGAGGACGATGCGGGCCATGGATGGTTTGGTTGGCTGTCCTCAGACGACGAGCGGGAAAAAAAA |
| 17 | PDGF | ATATATATGGGAGGACGATGCGGCAGCACCGAGGTACCCAACAGGGATCCGCCCAGACGACGAGCGGGAAAAAAAA |
| 18 | PDGF | ATATATATGGGAGGACGATGCGGGCGGCAGACGCGCCGGGTACCCCAGGTCCCCAGACGACGAGCGGGAAAAAAAA |
| 19 | PDGF | ATATATATGGGAGGACGATGCGGCACAAGGAACAAAGCGGCCCCTATCCCCAACAGACGACGAGCGGGAAAAAAAA |
| 20 | PDGF | ATATATATGGGAGGACGATGCGGGGGGCAAGAAGCACGGTACCCCAGGTCCGCCAGACGACGAGCGGGAAAAAAAA |
| 21 | PDGF | ATATATATGGGAGGACGATGCGGCCGGACATCCCCCAGGGCAAAACCAACTCCCAGACGACGAGCGGGAAAAAAAA |
| 22 | PDGF | ATATATATGGGAGGACGATGCGGCAAGGGAAACAGATAGCCCAGGCTCCCCCCCAGACGACGAGCGGGAAAAAAAA |
| 23 | Angiogenin | ATATATATGGGAGGACGATGCGGGCCAACCACGTGGTATTATTGACCTTGCAATGGGAATGCCCAGACGACGAGCGGGAAAAAAAA |
| 24 | Angiogenin | ATATATATGGGAGGACGATGCGGGGCAAACTGCGTCGTATTATAAGCCTCGCTACAGATGCCACAGACGACGAGCGGGAAAAAAAA |
| 25 | Angiogenin | ATATATATGGGAGGACGATGCGGGCACCTACCTGAGCTACATATGACAGTGTCACCCTGGCCCCAGACGACGAGCGGGAAAAAAAA |
| 26 | Angiogenin | ATATATATGGGAGGACGATGCGGGCCAAATGGACTTTTCGCCACGAACTTACGACGGTGTTGCCAGACGACGAGCGGGAAAAAAAA |
| 27 | Angiogenin | ATATATATGGGAGGACGATGCGGCACCAAAAGGTGGTCTTAGCCTAATTATGGACGTGTCCACCAGACGACGAGCGGGAAAAAAAA |
| 28 | Angiogenin | ATATATATGGGAGGACGATGCGGGCCACGTGTATTATCCTCAGCTTATAGCCATGGCATGGACCAGACGACGAGCGGGAAAAAAAA |
| 29 | Angiogenin | ATATATATGGGAGGACGATGCGGGCAAAGTCTTGGTCCACCAAATATGTGATGTCACCACCAGCAGACGACGAGCGGGAAAAAAAA |
| 30 | Angiogenin | ATATATATGGGAGGACGATGCGGGCCCTACTTGCATGAATATCCACTCCTAGGCTTGAGGGAGCAGACGACGAGCGGGAAAAAAAA |
| 31 | IL-4 | ATATATATGGGAGGACGATGCGGGCCGAAGTCTAAACCTGCTCGTGACTTTCTTTCGATGTTGCAGACGACGAGCGGGAAAAAAAA |
| 32 | JL-4 | ATATATATGGGAGGACGATGCGGGCCTACCAACTCCCCTCTAGTCCTGTTCTATCCACGTTGGCAGACGACGAGCGGGAAAAAAAA |
| 33 | IL-4 | ATATATATGGGAGGACGATGCGGGCCAAGGTTCCCTTCTGCCTCATTGTTGTCGGAACCCATCCAGACGACGAGCGGGAAAAAAAA |
| 34 | IL-4 | ATATATATGGGAGGACGATGCGGCCCCGAGTTTCCCTAAGGTTTGGTTGACCTGTCATTTCAGCAGACGACGAGCGGGAAAAAAAA |
| 35 | IL-4 | ATATATATGGGAGGACGATGCGGGCACAGGTTCTATCAACGTTGTCCTGAGTAATTGACCTGCAGACGACGAGCGGGAAAAAAAA |
| 36 | IL-4 | ATATATATGGGAGGACGATGCGGGCCAAGGACATTCTTGTTCGTTGTTGCTGTCCACTGTCTCCAGACGACGAGCGGGAAAAAAAA |
| 37 | β-NGF | ATATATATGGGAGGACGATGCGGGACCAATAACACTACACTGATCATCTCCCTTCTATGTCCCCAGACGACGAGCGGGAAAAAAAA |
| 38 | β-NGF | ATATATATGGGAGGACGATGCGGGCACAGTTAAATCCACTTCACCTTACAATTCCTTTATCTGCAGACGACGAGCGGGAAAAAAAA |
| 39 | β-NGF | ATATATATGGGAGGACGATGCGGCCATACGCACTTCAGTGGGGATAATCCAACTGGTTTGGTGCAGACGACGAGCGGGAAAAAAAA |
| 40 | β-NGF | ATATATATGGGAGGACGATGCGGGACCAAATACCAACTTCACATCACCTTTCTTATTCTCCGGCAGACGACGAGCGGGAAAAAAAA |
| 41 | β-NSF | ATATATATGGGAGGACGATGCGGGCACTAACTTTACCTCCACCTCTAACCACCCTCCTTTCTGCAGACGACGAGCGGGAAAAAAAA |
| 42 | β-NSF | ATATATATGGGAGGACGATGCGGGCCCCAAACACTTGTTCCTATCTTTCAACCCCCCTTGATCCAGACGACGAGCGGGAAAAAAAA |
| 43 | P-Selectin | ATATATATGGGAGGACGATGCGGCGCCCCGATTGACCTTCGATTTATCCTACTTATGGCACCCCAGACGACGAGCGGGAAAAAAAA |
| 44 | P-Selectin | ATATATATGGGAGGACGATGCGGCCATGAACCCATCCTCTGGTTCATAATCGACGTGTTCGTGCAGACGACGAGCGGGAAAAAAAA |
| 45 | P-Selectin | ATATATATGGGAGGACGATGCGGCACGAGGGAATCACCTCGAACTTGTCCTGGATTACTGCCCAGACGACGAGCGGGAAAAAAAA |
| 46 | P-Selectin | ATATATATGGGAGGACGATGCGGGCTCAATAACCTGAATCTACCTTTCCCTAGCAAAGGTCTGCAGACGACGAGCGGGAAAAAAAA |
| 47 | P-Selectin | ATATATATGGGAGGACGATGCGGCCATACGCACTTCAGTGGGGATAATCCAACTGGTTTGGTGCAGACGACGAGCGGGAAAAAAAA |
| 48 | TGF-β1 | ATATATATGGGAGGACGATGCGGGCACAACCTTACCACCCTAGCCTACCCCTAACCTCCTGTCCAGACGACGAGCGGGAAAAAAAA |
| 49 | TGF-β1 | ATATATATGGGAGGACGATGCGGGACCATCCAATACCTTCCGTAACACTTTCCTTCTTCCTTCCAGACGACGAGCGGGAAAAAAAA |
| 50 | TSF-β1 | ATATATATGGGAGGACGATGCGGGCAGCAACCTACCTTACCTTCCCCTAGCCTACCTTATCCCCAGACGACGAGCGGGAAAAAAAA |
| 51 | TSF-β1 | ATATATATGGGAGGACGATGCGGGCACCTTTCTTACATCTTGGCTTCATTCTTGCACCATTGGCAGACGACGAGCGGGAAAAAAAA |
| 52 | TSP-β1 | ATATATATGGGAGGACGATGCGGGCACAATCAAGACCTCTCCAAACTTGAACTCTGTCTATCCCAGACGACGAGCGGGAAAAAAAA |
| 53 | IL-7 | ATATATATGGGAGGACGATGCGGGCTGAAAGGAAACGGACGATTGAGCTTCCCCTTACCTCTCCAGACGACGAGCGGGAAAAAAAA |
| 54 | Kininogen | ATATATATGGGAGGACGATGCGGGACGCTAGTACCCTGGCTGGCTTGGTTGGGCAGACGACGAGCGGGAAAAAAAA |
| 55 | L-Selectin | ATATATATGGGAGGACGATGCGGCCGGTTCACGTGCACCATCCGTGTGCTAGACAGACGACGAGCGGGAAAAAAAA |
| 58 | Plasmin | ATATATATGGGAGGACGATGCGGCAACCCTGACACCACGTTGTTTCTCCTTTTGGGGTAACCGCAGACGACGAGCGGGAAAAAAAA |
| 57 | Serum | ATATATATGGGAGGACGATGCGGGCCGACTCTGAGGAAAAGGTTTTATGTATGGCTACCCCTGCAGACGACGAGCGGGAAAAAAAA |
| | amyloid P | |
| 58 | Tpo | ATATATATGGGAGGACGATGCGGGCACACCCAACCTTGCTTCTTCAATCTAATCTCCACTTTGCAGACGACGAGCGGGAAAAAAAA |
| 59 | Coagulation | ATATATATGGGAGGACGATGCGGGCGTCTGGGATTTGGACTTCTTCGCTAGCTCAGACGACGAGCGGGAAAAAAAA |
| | Factor IX | |
| 60 | Coagulation | ATATATATGGGAGGACGATGCGGCTGCGTGACAGTTATACTGTTATTGGTCTTCAGACGACGAGCGGGAAAAAAAA |
| | Factor XII | |
| 61 | Endostatin | ATATATATGGGAGGACGATGCGGCACAATGAAGTCACTCTTGACGCTTGTATTCAGACGACGAGCGGGAAAAAAAA |
| 62 | Factor H | ATATATATGGGAGGACGATGCGGCCTCATAAAGTTACATCGGCAATTCTTCTCCAGACGACGAGCGGGAAAAAAAA |
| 63 | Collagen | ATATATATGGGAGGACGATGCGGCTACTCCTCCTTAACCCGGGTCTTGTGGCCCAGACGACGAGCGGGAAAAAAAA |
| 64 | CTLA-4 Fc | ATATATATGGGAGGACGATGCGGGACGCTAATACTTCTGGAGTGGAACGGTTTCAGACGACGAGCGGGAAAAAAAA |
| 65 | HGF | ATATATATGGGAGGACGATGCGGGACGACTAGCCTAGTGCCCTTACGATCACCCAGACGACGAGCGGGAAAAAAAA |
| 66 | IGFBP-3 | ATATATATGGGAGGACGATGCGGGCAAAGTGTTATTTCTTGATCTGTTTCACCCAGACGACGAGCGGGAAAAAAAA |
| 67 | UGT 1A1 | ATATATATGGGAGGACGATGCGGCACCTGATTTCTACCCTTTACTTTGTGTGGCAGACGACGAGCGGGAAAAAAAA |
| 68 | UGT 1A10 | ATATATATGGGAGGACGATGCGGCACCACTTCTTTACCTCACTCTTTCTGCAGCAGACGACGAGCGGGAAAAAAAA |
| 69 | UGT 1A3 | ATATATATGGGAGGACGATGCGGGCCGACTTTGTCACCGAGTGCATCCGAGGTCAGACGACGAGCGGGAAAAAAAA |

What is claimed is:

1. A method for identifying photocrosslinking nucleic acid ligands of a target protein from a candidate mixture of nucleic acids wherein each member of said candidate mixture comprises one or more photoreactive groups, said method comprising:
a) contacting the candidate mixture with said target protein in solution, wherein nucleic acids having an increased affinity to said target protein relative to the candidate mixture form nucleic acid-target protein complexes;
b) irradiating said candidate mixture, wherein said nucleic acid-target protein complexes photocrosslink;
c) immobilizing said target protein on a solid support, whereby said photocrosslinked nucleic acid-target protein complexes are immobilized on said solid support;
d) partitioning said solid support from the remainder of the candidate mixture whereby immobilized photocrosslinked nucleic acid-target protein complexes are partitioned from the remainder of the candidate mixture;
whereby a photocrosslinking nucleic acid ligand of said target protein is identified.
e) amplifying the nucleic acids that photocrosslinked to the target protein to yield a mixture of nucleic acids enriched in sequences that are capable of photocrosslinking the target protein; whereby a photocrosslinking nucleic acid ligand of said target protein is identified.

2. The method of claim 1 further comprising the step:
f) repeating steps a) through e) using the enriched mixture of each successive repeat as many times as required to yield a desired level of increased enrichment; whereby a photocrosslinking nucleic acid ligand of said target protein is identified.

3. The method of claim 1 wherein said photocrosslinking nucleic acid ligand is a single-stranded nucleic acid.

4. The method of claim 3 wherein said single-stranded nucleic acid is ribonucleic acid.

5. The method of claim 3 wherein said single-stranded nucleic acid is deoxyribonucleic acid.

6. The method of claim 1 wherein said candidate mixture further comprises fixed sequence regions, and wherein the amplification is performed using the polymerase chain reaction (PCR) with primers complementary to said fixed sequence regions, wherein the 5′ ends of said primers are attached to tail sequences having a lower melting temperature (Tm) than said primers, wherein the polymerase chain reaction comprises a denaturation step, a primer annealing step, and a primer extension step, and wherein said primer annealing step and said primer extension step are performed at a temperature higher than the melting temperature of said tail sequences.

7. The method of claim 1 wherein said photoreactive groups are selected from the group consisting of 5-bromouracil, 5-iodouracil, 5-bromovinyluracil, 5-iodovinyluracil, 5-azidouracil, 4-thiouracil, 5-bromocytosine, 5-iodocytosine, 5-bromovinylcytosine, 5-iodovinylcytosine, 5-azidocytosine, 8-azidoadenine, 8-bromoadenine, 8-iodoadenine, 8-azidoguanine, 8-bromoguanine, 8-iodoguanine, 8-azidohypoxanthine, 8-bromohypoxanthine, 8-iodohypoxanthine, 8-azidoxanthine, 8-bromoxanthine, 8-iodoxanthine, 5-bromodeoxyuracil, 8-bromo-2′-deoxyadenine, 5-iodo-2′-deoxyuracil, 5-iodo-2′-deoxycytosine, 5-[(4-azidophenacyl)thio]cytosine, 5-[(4-azidophenacyl)thio]uracil, 7-deaza-7-iodoadenine, 7-deaza-7-iodoguanine, 7-deaza-7-bromoadenine, and 7-deaza-7-bromoguanine.

8. The method of claim 1 further comprising releasing nucleic acids from the immobilized photocrosslinked nucleic acid-target protein complexes by proteolytic digestion.

9. The method of claim 1 further comprising washing the partitioned solid support under conditions selected from the group consisting of nucleic acid denaturing conditions, protein denaturing conditions, and protein and nucleic acid denaturing conditions.

10. The method of claim 1 wherein said solid support is derivatized with tosyl groups, and wherein said nucleic acid-target protein complexes are immobilized on said solid support through the reaction of the target protein with said tosyl groups.

11. The method of claim 1 wherein said solid support is a bead.

12. The method of claim 11 wherein said bead is a paramagnetic bead.

13. The method of claim 1 wherein said solid support is a multi-well microtiter plate.

14. The method of claim 1 wherein steps a) through e) are carried out by automated machines controlled by a computer.

15. The method of claim 1 wherein steps a) through d) are carried out by automated machines controlled by a computer.

16. A method for identifying photocrosslinking nucleic acid ligands of a target protein from a candidate mixture of nucleic acids wherein each member of said candidate mixture comprises one or more photoreactive groups, said method comprising:
a) contacting the candidate mixture with said target protein in solution, wherein nucleic acids having an increased affinity to said protein target relative to the candidate mixture form nucleic acid-target protein complexes;
b) irradiating said candidate mixture, wherein said nucleic acid-target protein complexes photocrosslink;
c) contacting said candidate mixture with a tosyl-derivatized solid support whereby nucleic acid-target protein complexes become immobilized on said tosyl-derivatized solid support through the reaction of the tosyl groups with said target protein;
d) partitioning the tosyl-derivatized solid supports from the candidate mixture;
e) releasing nucleic acids from said tosyl-derivatized solid by proteolytic digestion;
f) amplifying the nucleic acids released in e) to yield a mixture of nucleic acids enriched in sequences that are capable of photocrosslinking to the target protein; and
g) repeating steps a) through f) using the enriched mixture of each successive repeat as many times as required to yield a desired level of enrichment for sequence capable of photocrosslinking to the target protein, whereby a photocrosslinking nucleic acid ligand of the target is identified.

17. The method of claim 16 further comprising the step of:
washing the nucleic acid-target protein complexed tosyl-derivatized solid supports under conditions selected from the group consisting of nucleic acid denaturing conditions, protein denaturing conditions, and protein and nucleic acid denaturing conditions.

18. The method of claim 16 wherein said candidate mixture further comprises fixed sequence regions, and wherein the amplification is performed using the polymerase chain reaction (PCR) with primers complementary to said fixed sequence regions, wherein the 5′ ends of said primers are attached to tail sequences having a lower melting temperature (Tm) than said primers, wherein the polymerase chain reaction comprises a denaturation step, a primer annealing step, and a primer extension step, and wherein said primer annealing step and said primer extension step are performed at a temperature higher than the melting temperature of said tail sequences.

19. The method of claim 16 wherein steps a) through e) are carried out by automated machines controlled by a computer.

20. The method of claim 16 wherein steps a) through f) are carried out by automated machines controlled by a computer.

21. A method for identifying photocrosslinking nucleic acid ligands of a target protein from a candidate mixture of nucleic acids wherein each member of said candidate mixture comprises one or more photoreactive groups, said method comprising:
a) contacting the candidate mixture with the target protein, wherein nucleic acids having an increased affinity to the target relative to the candidate mixture may be partitioned from the remainder of the candidate mixture;
b) partitioning the increased affinity nucleic acids from the remainder of the candidate mixture;
c) amplifying the increased affinity nucleic acids to yield a ligand-enriched mixture of nucleic acids;
d) contacting said ligand-enriched mixture with the target protein in solution, wherein nucleic acid-target complexes form;
e) irradiating said candidate mixture, wherein said nucleic acid-target complexes photocrosslink;
f) immobilizing said target protein on a solid support, whereby said photocrosslinked nucleic acid-target protein complexes are immobilized on said solid support;
g) partitioning said solid support with immobilized photocrosslinked nucleic acid-target complexes from the remainder of the candidate mixture; and
h) amplifying the nucleic acids that photocrosslinked to the target protein to yield a mixture of nucleic acids enriched in sequences that are capable of photocrosslinking the target protein, whereby a photocrosslinking nucleic acid ligand of said target protein is identified.

22. The method of claim 21 further comprising after step c):
I. repeating steps a)-c) using the ligand-enriched mixture of each successive repeat as many times as required to yield a desired level of ligand enrichment.

23. The method of claim 21 further comprising the step:
i) repeating steps d) through h) using the mixture of nucleic acids enriched in sequences that are capable of photocrosslinking the target protein of each successive repeat as many times as required to yield a desired level of increased ligand enrichment; whereby a photocrosslinking nucleic acid ligand of said target protein is identified.

24. The method of claim 21 wherein in step a) said target protein is associated with a solid support.

25. The method of claim 24 wherein said solid support in step a) is a paramagnetic bead.

26. The method of claim 24 wherein said solid support in step a) is a microtiter plate.

27. The method of claim 21 wherein said partitioning step b) employs filter binding selection.

28. The method of claim 21, wherein said solid support in step f) is a paramagnetic bead.

29. The method of claim 21 wherein said solid support in step f) is derivatized with tosyl groups, whereby said nucleic acid-target complexes become immobilized on said solid support via the reaction between target protein and said tosyl groups.

30. The method of claim 21 further comprising releasing nucleic acids from the immobilized photocrosslinked nucleic acid-target protein complexes by proteolytic digestion.

31. The method of claim 21 further comprising washing the partitioned solid supports with immobilized photocrosslinked nucleic acid-target complexes under conditions selected from the group consisting of nucleic acid denaturing conditions, protein denaturing conditions, and protein and nucleic acid denaturing conditions.

32. The method of claim 21 wherein said candidate mixture further comprises fixed sequence regions, and wherein the amplification is performed using the polymerase chain reaction (PCR) with primers complementary to said fixed sequence regions, wherein the 5′ ends of said primers are attached to tail sequences having a lower melting temperature (Tm) than said primers, wherein the polymerase chain reaction comprises a denaturation step, a primer annealing step, and a primer extension step, and wherein said primer annealing step and said primer extension step are performed at a temperature higher than the melting temperature of said tail sequences.

33. The method of claim 21 wherein steps a)-c) are carried out by automated machines controlled by a computer.

34. The method of claim 21 wherein steps a)-b) are carried out by automated machines controlled by a computer.

35. The method of claim 21 wherein steps d)-h) are carried out by automated machines controlled by a computer.

36. The method of claim 21 wherein steps d)-g) are carried out by automated machines controlled by a computer.

37. A non-naturally occurring photocrosslinking nucleic acid ligand to a protein target selected from the group consisting of human neutrophil elastase (hNE), HIV-1_{MN} gp120, human L-selectin, human P-Selectin, human platelet-derived growth factor (PDGF), human alpha-thrombin, human basic fibroblast growth factor (bFGF), HIV-1MN gp120, Angiogenin, Interleukin-4 (IL-4), β-Nerve Growth Factor (β-NGF), Transforming Growth Factor β1 (TGF-β1), Interleukin-7 (IL-7), Kininogen, Plasmin, Serum Amyloid P, Thrombopoietin (Tpo), Coagulation Factor IX, Coagulation Factor XII, Endostatin, Factor H, Collagen, Cytotoxic T lymphocyte-associated protein-4 Fc (CTLA-4 Fe), Hepatocyte Growth Factor (HGF), Insulin-like growth factor binding protein-3 (IGFBP-3), UDP-glucuronosyl transferase (UGT) 1A1, UGT 1A1, and UGT 1A3.

38. The purified and isolated non-naturally occurring photocrosslinking nucleic acid ligand of claim 37 wherein said photocrosslinking nucleic acid ligand is a photocrosslinking nucleic acid ligand to human neutrophil elastase (hNE).

39. The photocrosslinking nucleic acid ligand of claim 38, wherein said photocrosslinking nucleic acid ligand is selected from the group consisting of SEQ ID NOS:1-3.

40. The photocrosslinking nucleic acid ligand of claim 38 wherein said photocrosslinking nucleic acid ligand is substantially homologous to a photocrosslinking nucleic acid ligand selected from the group consisting of SEQ ID NOS:1-3.

41. The photocrosslinking nucleic acid ligand of claim 38 wherein said photocrosslinking nucleic acid ligand has substantially the same structure as a photocrosslinking nucleic acid ligand selected from the group consisting of SEQ ID NOS:1-3.

42. The purified and isolated non-naturally occurring photocrosslinking nucleic acid ligand of claim 37 wherein said photocrosslinking nucleic acid ligand is a photocrosslinking nucleic acid ligand to thrombin.

43. The photocrosslinking nucleic acid ligand of claim 42, wherein said photocrosslinking nucleic acid ligand is selected from the group consisting of SEQ ID NOS:9-12.

44. The photocrosslinking nucleic acid ligand of claim 42 wherein said photocrosslinking nucleic acid ligand is substantially homologous to a photocrosslinking nucleic acid ligand selected from the group consisting of SEQ ID NOS:9-12.

45. The photocrosslinking nucleic acid ligand of claim 42 wherein said photocrosslinking nucleic acid ligand has substantially the same structure as a photocrosslinking nucleic acid ligand selected from the group consisting of SEQ ID NOS:9-12.

46. The purified and isolated non-naturally occurring photocrosslinking nucleic acid ligand of claim 37 wherein said photocrosslinking nucleic acid ligand is a photocrosslinking nucleic acid ligand to HIV-1_{MAN} gp120.

47. The photocrosslinking nucleic acid ligand of claim 46, wherein said photocrosslinking nucleic acid ligand is selected from the group consisting of SEQ ID NOS:4-5 and SEQ ID NOS:13-16.

48. The photocrosslinking nucleic acid ligand of claim 46 wherein said photocrosslinking nucleic acid ligand is substantially homologous to a photocrosslinking nucleic acid ligand selected from the group consisting of SEQ ID NOS:4-5 and SEQ ID NOS:13-16.

49. The photocrosslinking nucleic acid ligand of claim 46 wherein said photocrosslinking nucleic acid ligand has substantially the same structure as a photocrosslinking nucleic acid ligand selected from the group consisting of SEQ ID NOS:4-5 and SEQ ID NOS:13-16.

50. The purified and isolated non-naturally occurring photocrosslinking nucleic acid ligand of claim 37 wherein said photocrosslinking nucleic acid ligand is a photocrosslinking nucleic acid ligand to human platelet-derived growth factor (PDGF).

51. The photocrosslinking nucleic acid ligand of claim 50, wherein said photocrosslinking nucleic acid ligand is selected from the group consisting of SEQ ID NOS:6-8 and SEQ ID. NOS. 17-22

52. The photocrosslinking nucleic acid ligand of claim 50 wherein said photocrosslinking nucleic acid ligand is substantially homologous to a photocrosslinking nucleic acid ligand selected from the group consisting of SEQ ID NOS:6-8 and SEQ ID NOS:17-22.

53. The photocrosslinking nucleic acid ligand of claim 50 wherein said photocrosslinking nucleic acid ligand has substantially the same structure as a photocrosslinking nucleic acid ligand selected from the group consisting of SEQ ID NOS:6-8 and SEQ ID NOS:17-22.

54. The purified and isolated non-naturally occurring photocrosslinking nucleic acid ligand of claim 37 wherein said photocrosslinking nucleic acid ligand is a photocrosslinking nucleic acid ligand to angiogenin.

55. The photocrosslinking nucleic acid ligand of claim 54, wherein said photocrosslinking nucleic acid ligand is selected from the group consisting of SEQ ID NOS:23-30.

56. The photocrosslinking nucleic acid ligand of claim 54 wherein said photocrosslinking nucleic acid ligand is substantially homologous to a photocrosslinking nucleic acid ligand selected from the group consisting of SEQ ID NOS:23-30.

57. The photocrosslinking nucleic acid ligand of claim 54 wherein said photocrosslinking nucleic acid ligand has substantially the same structure as a photocrosslinking nucleic acid ligand selected from the group consisting of SEQ ID NOS:23-30.

58. The purified and isolated non-naturally occurring photocrosslinking nucleic acid ligand of claim 37 wherein said photocrosslinking nucleic acid ligand is a photocrosslinking nucleic acid ligand to Interleukin-4 (IL-4).

59. The photocrosslinking nucleic acid ligand of claim 58, wherein said photocrosslinking nucleic acid ligand is selected from the group consisting of SEQ ID NOS:31-36.

60. The photocrosslinking nucleic acid ligand of claim 58 wherein said photocrosslinking nucleic acid ligand is substantially homologous to a photocrosslinking nucleic acid ligand selected from the group consisting of SEQ ID NOS:31-36.

61. The photocrosslinking nucleic acid ligand of claim 58 wherein said photocrosslinking nucleic acid ligand has substantially the same structure as a photocrosslinking nucleic acid ligand selected from the group consisting of SEQ ID NOS:31-36.

62. The purified and isolated non-naturally occurring photocrosslinking nucleic acid ligand of claim 37 wherein said photocrosslinking nucleic acid ligand is a photocrosslinking nucleic acid ligand to β-Nerve Growth Factor (β-NGF).

63. The photocrosslinking nucleic acid ligand of claim 62, wherein said photocrosslinking nucleic acid ligand is selected from the group consisting of SEQ ID NOS:37-42.

64. The photocrosslinking nucleic acid ligand of claim 62 wherein said photocrosslinking nucleic acid ligand is substantially homologous to a photocrosslinking nucleic acid ligand selected from the group consisting of SEQ ID NOS:37-42.

65. The photocrosslinking nucleic acid ligand of claim 62 wherein said photocrosslinking nucleic acid ligand has substantially the same structure as a photocrosslinking nucleic acid ligand selected from the group consisting of SEQ ID NOS:37-42.

66. The purified and isolated non-naturally occurring photocrosslinking nucleic acid ligand of claim 37 wherein said photocrosslinking nucleic acid ligand is a photocrosslinking nucleic acid ligand to human P-Selectin.

67. The photocrosslinking nucleic acid ligand of claim 66, wherein said photocrosslinking nucleic acid ligand is selected from the group consisting of SEQ ID NOS:43-47.

68. The photocrosslinking nucleic acid ligand of claim 66 wherein said photocrosslinking nucleic acid ligand is substantially homologous to a photocrosslinking nucleic acid ligand selected from the group consisting of SEQ ID NOS:43-47.

69. The photocrosslinking nucleic acid ligand of claim 66 wherein said photocrosslinking nucleic acid ligand has substantially the same structure as a photocrosslinking nucleic acid ligand selected from the group consisting of SEQ ID NOS:43-47.

70. The purified and isolated non-naturally occurring photocrosslinking nucleic acid ligand of claim 37 wherein said photocrosslinking nucleic acid ligand is a photocrosslinking nucleic acid ligand to Transforming Growth Factor β1 (TGF-β1).

71. The photocrosslinking nucleic acid ligand of claim 70, wherein said photocrosslinking nucleic acid ligand is selected from the group consisting of SEQ ID NOS:48-52.

72. The photocrosslinking nucleic acid ligand of claim 70 wherein said photocrosslinking nucleic acid ligand is substantially homologous to a photocrosslinking nucleic acid ligand selected from the group consisting of SEQ ID NOS:48-52.

73. The photocrosslinking nucleic acid ligand of claim 70 wherein said photocrosslinking nucleic acid ligand has substantially the same structure as a photocrosslinking nucleic acid ligand selected from the group consisting of SEQ ID NOS:48-52.

74. The purified and isolated non-naturally occurring photocrosslinking nucleic acid ligand of claim 37 wherein said photocrosslinking nucleic acid ligand is a photocrosslinking nucleic acid ligand to Interleukin-7.

75. The photocrosslinking nucleic acid ligand of claim 74, wherein said photocrosslinking nucleic acid ligand comprises the sequence shown in SEQ ID NO:53.

76. The photocrosslinking nucleic acid ligand of claim 74 wherein said photocrosslinking nucleic acid ligand is substantially homologous to a photocrosslinking nucleic acid ligand comprising the sequence shown in SEQ ID NO:53.

77. The photocrosslinking nucleic acid ligand of claim 74 wherein said photocrosslinking nucleic acid ligand has substantially the same structure as a photocrosslinking nucleic acid ligand comprising the sequence shown in SEQ ID NO:53.

78. The purified and isolated non-naturally occurring photocrosslinking nucleic acid ligand of claim 37 wherein said photocrosslinking nucleic acid ligand is a photocrosslinking nucleic acid ligand to Kininogen.

79. The photocrosslinking nucleic acid ligand of claim 78, wherein said photocrosslinking nucleic acid ligand comprises the sequence shown in SEQ ID NO:54.

80. The photocrosslinking nucleic acid ligand of claim 78 wherein said photocrosslinking nucleic acid ligand is substantially homologous to a photocrosslinking nucleic acid ligand comprising the sequence shown in SEQ ID NO:54.

81. The photocrosslinking nucleic acid ligand of claim 78 wherein said photocrosslinking nucleic acid ligand has substantially the same structure as a photocrosslinking nucleic acid ligand comprising the sequence shown in SEQ ID NO:54.

82. The purified and isolated non-naturally occurring photocrosslinking nucleic acid ligand of claim 37 wherein said photocrosslinking nucleic acid ligand is a photocrosslinking nucleic acid ligand to L-Selectin.

83. The photocrosslinking nucleic acid ligand of claim 82, wherein said photocrosslinking nucleic acid ligand comprises the sequence shown in SEQ ID NO:55.

84. The photocrosslinking nucleic acid ligand of claim 82 wherein said photocrosslinking nucleic acid ligand is substantially homologous to a photocrosslinking nucleic acid ligand comprising the sequence shown in SEQ ID NO:55.

85. The photocrosslinking nucleic acid ligand of claim 82 wherein said photocrosslinking nucleic acid ligand has substantially the same structure as a photocrosslinking nucleic acid ligand comprising the sequence shown in SEQ ID NO:55.

86. The purified and isolated non-naturally occurring photocrosslinking nucleic acid ligand of claim 37 wherein said photocrosslinking nucleic acid ligand is a photocrosslinking nucleic acid ligand to Plasmin.

87. The photocrosslinking nucleic acid ligand of claim 86, wherein said photocrosslinking nucleic acid ligand comprises the sequence shown in SEQ ID NO:56.

88. The photocrosslinking nucleic acid ligand of claim 86 wherein said photocrosslinking nucleic acid ligand is substantially homologous to a photocrosslinking nucleic acid ligand comprising the sequence shown in SEQ ID NO:56.

89. The photocrosslinking nucleic acid ligand of claim 86 wherein said photocrosslinking nucleic acid ligand has substantially the same structure as a photocrosslinking nucleic acid ligand comprising the sequence shown in SEQ ID NO:56.

90. The purified and isolated non-naturally occurring photocrosslinking nucleic acid ligand of claim 37 wherein said photocrosslinking nucleic acid ligand is a photocrosslinking nucleic acid ligand to Serum Amyloid P.

91. The photocrosslinking nucleic acid ligand of claim 90, wherein said photocrosslinking nucleic acid ligand comprises the sequence shown in SEQ ID NO:57.

92. The photocrosslinking nucleic acid ligand of claim 90 wherein said photocrosslinking nucleic acid ligand is substantially homologous to a photocrosslinking nucleic acid ligand comprising the sequence shown in SEQ ID NO:57.

93. The photocrosslinking nucleic acid ligand of claim 90 wherein said photocrosslinking nucleic acid ligand has substantially the same structure as a photocrosslinking nucleic acid ligand comprising the sequence shown in SEQ ID NO:57.

94. The purified and isolated non-naturally occurring photocrosslinking nucleic acid ligand of claim 37 wherein said photocrosslinking nucleic acid ligand is a photocrosslinking nucleic acid ligand to Thrombopoietin (Tpo).

95. The photocrosslinking nucleic acid ligand of claim 94, wherein said photocrosslinking nucleic acid ligand comprises the sequence shown in SEQ ID NO:58.

96. The photocrosslinking nucleic acid ligand of claim 94 wherein said photocrosslinking nucleic acid ligand is substantially homologous to a photocrosslinking nucleic acid ligand comprising the sequence shown in SEQ ID NO:58.

97. The photocrosslinking nucleic acid ligand of claim 94 wherein said photocrosslinking nucleic acid ligand has substantially the same structure as a photocrosslinking nucleic acid ligand comprising the sequence shown in SEQ ID NO:58.

98. The purified and isolated non-naturally occurring photocrosslinking nucleic acid ligand of claim 37 wherein said photocrosslinking nucleic acid ligand is a photocrosslinking nucleic acid ligand to Coagulation Factor IX.

99. The photocrosslinking nucleic acid ligand of claim 98, wherein said photocrosslinking nucleic acid ligand comprises the sequence shown in SEQ ID NO:59.

100. The photocrosslinking nucleic acid ligand of claim 98 wherein said photocrosslinking nucleic acid ligand is substantially homologous to a photocrosslinking nucleic acid ligand comprising the sequence shown in SEQ ID NO:59.

101. The photocrosslinking nucleic acid ligand of claim 98 wherein said photocrosslinking nucleic acid ligand has substantially the same structure as a photocrosslinking nucleic acid ligand comprising the sequence shown in SEQ ID NO:59.

102. The purified and isolated non-naturally occurring photocrosslinking nucleic acid ligand of claim 37 wherein said photocrosslinking nucleic acid ligand is a photocrosslinking nucleic acid ligand to Coagulation Factor XII.

103. The photocrosslinking nucleic acid ligand of claim 102, wherein said photocrosslinking nucleic acid ligand comprises the sequence shown in SEQ ID NO:60.

104. The photocrosslinking nucleic acid ligand of claim 102 wherein said photocrosslinking nucleic acid ligand is substantially homologous to a photocrosslinking nucleic acid ligand comprising the sequence shown in SEQ ID NO:60.

105. The photocrosslinking nucleic acid ligand of claim 102 wherein said photocrosslinking nucleic acid ligand has substantially the same structure as a photocrosslinking nucleic acid ligand comprising the sequence shown in SEQ ID NO:60.

106. The purified and isolated non-naturally occurring photocrosslinking nucleic acid ligand of claim 37 wherein said photocrosslinking nucleic acid ligand is a photocrosslinking nucleic acid ligand to Endostatin.

107. The photocrosslinking nucleic acid ligand of claim 106, wherein said photocrosslinking nucleic acid ligand comprises the sequence shown in SEQ ID NO:61.

108. The photocrosslinking nucleic acid ligand of claim 106 wherein said photocrosslinking nucleic acid ligand is substantially homologous to a photocrosslinking nucleic acid ligand comprising the sequence shown in SEQ ID NO:61.

109. The photocrosslinking nucleic acid ligand of claim 106 wherein said photocrosslinking nucleic acid ligand has substantially the same structure as a photocrosslinking nucleic acid ligand comprising the sequence shown in SEQ ID NO:61.

110. The purified and isolated non-naturally occurring photocrosslinking nucleic acid ligand of claim 37 wherein said photocrosslinking nucleic acid ligand is a photocrosslinking nucleic acid ligand to Factor H.

111. The photocrosslinking nucleic acid ligand of claim 110, wherein said photocrosslinking nucleic acid ligand comprises the sequence shown in SEQ ID NO:62.

112. The photocrosslinking nucleic acid ligand of claim 110 wherein said photocrosslinking nucleic acid ligand is substantially homologous to a photocrosslinking nucleic acid ligand comprising the sequence shown in SEQ ID NO:62.

113. The photocrosslinking nucleic acid ligand of claim 110 wherein said photocrosslinking nucleic acid ligand has substantially the same structure as a photocrosslinking nucleic acid ligand comprising the sequence shown in SEQ ID NO:62.

114. The purified and isolated non-naturally occurring photocrosslinking nucleic acid ligand of claim 37 wherein said photocrosslinking nucleic acid ligand is a photocrosslinking nucleic acid ligand to Collagen.

115. The photocrosslinking nucleic acid ligand of claim 114, wherein said photocrosslinking nucleic acid ligand comprises the sequence shown in SEQ ID NO:63.

116. The photocrosslinking nucleic acid ligand of claim 114 wherein said photocrosslinking nucleic acid ligand is substantially homologous to a photocrosslinking nucleic acid ligand comprising the sequence shown in SEQ ID NO:63.

117. The photocrosslinking nucleic acid ligand of claim 114 wherein said photocrosslinking nucleic acid ligand has substantially the same structure as a photocrosslinking nucleic acid ligand comprising the sequence shown in SEQ ID NO:63.

118. The purified and isolated non-naturally occurring photocrosslinking nucleic acid ligand of claim 37 wherein said photocrosslinking nucleic acid ligand is a photocrosslinking nucleic acid ligand to Cytotoxic T lymphocyte-associated protein-4 Fc (CTLA-4 Fc).

119. The photocrosslinking nucleic acid ligand of claim 118, wherein said photocrosslinking nucleic acid ligand comprises the sequence shown in SEQ ID NO:64.

120. The photocrosslinking nucleic acid ligand of claim 118 wherein said photocrosslinking nucleic acid ligand is substantially homologous to a photocrosslinking nucleic acid ligand comprising the sequence shown in SEQ ID NO:64.

121. The photocrosslinking nucleic acid ligand of claim 118 wherein said photocrosslinking nucleic acid ligand has substantially the same structure as a photocrosslinking nucleic acid ligand comprising the sequence shown in SEQ ID NO:64.

122. The purified and isolated non-naturally occurring photocrosslinking nucleic acid ligand of claim 37 wherein said photocrosslinking nucleic acid ligand is a photocrosslinking nucleic acid ligand to Hepatocyte Growth Factor (HGF).

123. The photocrosslinking nucleic acid ligand of claim 122, wherein said photocrosslinking nucleic acid ligand comprises the sequence shown in SEQ ID NO:65.

124. The photocrosslinking nucleic acid ligand of claim 122 wherein said photocrosslinking nucleic acid ligand is substantially homologous to a photocrosslinking nucleic acid ligand comprising the sequence shown in SEQ ID NO:65.

125. The photocrosslinking nucleic acid ligand of claim 122 wherein said photocrosslinking nucleic acid ligand has substantially the same structure as a photocrosslinking nucleic acid ligand comprising the sequence shown in SEQ ID NO:65.

126. The purified and isolated non-naturally occurring photocrosslinking nucleic acid ligand of claim 37 wherein said photocrosslinking nucleic acid ligand is a photocrosslinking nucleic acid ligand to Insulin-like growth factor binding protein-3 (IGFBP-3).

127. The photocrosslinking nucleic acid ligand of claim 126, wherein said photocrosslinking nucleic acid ligand comprises the sequence shown in SEQ ID NO:66.

128. The photocrosslinking nucleic acid ligand of claim 126 wherein said photocrosslinking nucleic acid ligand is substantially homologous to a photocrosslinking nucleic acid ligand comprising the sequence shown in SEQ ID NO:66.

129. The photocrosslinking nucleic acid ligand of claim 126 wherein said photocrosslinking nucleic acid ligand has substantially the same structure as a photocrosslinking nucleic acid ligand comprising the sequence shown in SEQ ID NO:66.

130. The purified and isolated non-naturally occurring photocrosslinking nucleic acid ligand of claim 37 wherein said photocrosslinking nucleic acid ligand is a photocrosslinking nucleic acid ligand to UDP-glucuronosyl transferase (UGT) 1A1.

131. The photocrosslinking nucleic acid ligand of claim 130, wherein said photocrosslinking nucleic acid ligand comprises the sequence shown in SEQ ID NO:67.

132. The photocrosslinking nucleic acid ligand of claim 130 wherein said photocrosslinking nucleic acid ligand is substantially homologous to a photo crosslinkidng nucleic acid ligand comprising the sequence shown in SEQ ID NO:67.

133. The photocrosslinking nucleic acid ligand of claim 130 wherein said photocrosslinking nucleic acid ligand has substantially the same structure as a photocrosslinking nucleic acid ligand comprising the sequence shown in SEQ ID NO:67.

134. The purified and isolated non-naturally occurring photocrosslinking nucleic acid ligand of claim 37 wherein said photocrosslinking nucleic acid ligand is a photocrosslinking nucleic acid ligand to UDP-glucuronosyl transferase (UGT) 1A10.

135. The photocrosslinking nucleic acid ligand of claim 134, wherein said photocrosslinking nucleic acid ligand comprises the sequence shown in SEQ ID NO:68.

136. The photocrosslinking nucleic acid ligand of claim 134 wherein said photocrosslinking nucleic acid ligand is substantially homologous to a photocrosslinking nucleic acid ligand comprising the sequence shown in SEQ ID NO:68.

137. The photocrosslinking nucleic acid ligand of claim 134 wherein said photocrosslinking nucleic acid ligand has substantially the same structure as a photocrosslinking nucleic acid ligand comprising the sequence shown in SEQ ID NO:68.

138. The purified and isolated non-naturally occurring photocrosslinking nucleic acid ligand of claim 37 wherein said photocrosslinking nucleic acid ligand is a photocrosslinking nucleic acid ligand to UDP-glucuronosyl transferase (UGT) 1A3.

139. The photocrosslinking nucleic acid ligand of claim 138, wherein said photocrosslinking nucleic acid ligand comprises the sequence shown in SEQ ID NO:69.

140. The photocrosslinking nucleic acid ligand of claim 138 wherein said photocrosslinking nucleic acid ligand is substantially homologous to a photocrosslinking nucleic acid ligand comprising the sequence shown in SEQ ID NO:69.

141. The photocrosslinking nucleic acid ligand of claim 138 wherein said photocrosslinking nucleic acid ligand has substantially the same structure as a photocrosslinking nucleic acid ligand comprising the sequence shown in SEQ ID NO:69.

142. The non-naturally occurring photocrosslinking nucleic acid ligand of claim 37 wherein said photocrosslinking nucleic acid ligand is identified by the method of any one of claims 1, 16, or 21.

143. An automated machine for identifying photocrosslinking nucleic acid ligands of a target protein from a candidate mixture of nucleic acids wherein each member of said candidate mixture comprises photoreactive groups, wherein said automated machine performs the steps of:
a) contacting the candidate mixture with said target protein in solution, wherein nucleic acids having an increased affinity to said target protein relative to the candidate mixture form nucleic acid-target protein complexes;
b) irradiating said candidate mixture, wherein said nucleic acid-target protein complexes photocrosslink;
c) immobilizing said target protein on a solid support, whereby said photocrosslinked nucleic acid-target protein complexes are immobilized on said solid support;
d) partitioning said solid support from the remainder of the candidate mixture whereby immobilized photocrosslinked nucleic acid-target protein complexes are partitioned from the remainder of the candidate mixture;
whereby a photocrosslinking nucleic acid ligand of said target protein is identified following amplification of the nucleic acids that photocrosslinked to the target protein to yield a mixture of nucleic acids enriched in sequences that are capable of photocrosslinking the target protein;
and wherein steps a)-d) are performed at one or more work stations on a work surface by a robotic manipulator controlled by a computer.

144. An automated machine for identifying photocrosslinking nucleic acid ligands of a target protein from a candidate mixture of nucleic acids wherein each member of said candidate mixture comprises photoreactive groups, wherein said automated machine performs the steps of:
a) contacting the candidate mixture with said target protein in solution, wherein nucleic acids having an increased affinity to said target protein relative to the candidate mixture form nucleic acid-target protein complexes;
b) irradiating said candidate mixture, wherein said nucleic acid-target protein complexes photocrosslink;
c) immobilizing said target protein on a solid support, whereby said photocrosslinked nucleic acid-target protein complexes are immobilized on said solid support;
d) partitioning said solid support from the remainder of the candidate mixture whereby immobilized photocrosslinked nucleic acid-target protein complexes are partitioned from the remainder of the candidate mixture;
e) amplifying the nucleic acids that photocrosslinked to the target protein to yield a mixture of nucleic acids enriched in sequences that are capable of photocrosslinking the target protein; whereby a photocrosslinking nucleic acid ligand of said target protein is identified;
wherein steps a)-e) are performed at one or more work stations on a work surface by a robotic manipulator controlled by a computer.

145. An automated machine for identifying photocrosslinking nucleic acid ligands of a target protein from a candidate mixture of nucleic acids wherein each member of said candidate mixture comprises photoreactive groups, said automated machine comprising:
a) means for contacting the candidate mixture with said target protein in solution, wherein nucleic acids having an increased affinity to said target protein relative to the candidate mixture form nucleic acid-target protein complexes;
b) means for irradiating said candidate mixture, wherein said nucleic acid-target protein complexes photocrosslink;
c) means for immobilizing said target protein on a solid support, whereby said photocrosslinked nucleic acid-target protein complexes are immobilized on said solid support;
d) means for partitioning said solid support from the remainder of the candidate mixture whereby immobilized photocrosslinked nucleic acid-target protein complexes are partitioned from the remainder of the candidate mixture; whereby a photocrosslinking nucleic acid ligand of said target protein is identified following amplification of the nucleic acids that photocrosslinked to the target protein to yield a mixture of nucleic acids enriched in sequences that are capable of photocrosslinking the target protein;
wherein steps a)-d) are performed at one or more work stations on a work surface by a robotic manipulator controlled by a computer.

146. An automated machine for identifying photocrosslinking nucleic acid ligands of a target protein from a candidate mixture of nucleic acids wherein each member of said candidate mixture comprises photoreactive groups, said automated machine comprising:
a) means for contacting the candidate mixture with said target protein in solution, wherein nucleic acids having an increased affinity to said target protein relative to the candidate mixture form nucleic acid-target protein complexes;
b) means for irradiating said candidate mixture, wherein said nucleic acid-target protein complexes photocrosslink;
c) means for immobilizing said target protein on a solid support, whereby said photocrosslinked nucleic acid-target protein complexes are immobilized on said solid support;
d) means for partitioning said solid support from the remainder of the candidate mixture whereby immobilized photocrosslinked nucleic acid-target protein complexes are partitioned from the remainder of the candidate mixture
e) means for amplifying the nucleic acids that photocrosslinked to the target protein to yield a mixture of nucleic acids enriched in sequences that are capable of photocrosslinking the target protein; whereby a photocrosslinking nucleic acid ligand of said target protein is identified;
wherein steps a)-e) are performed at one or more work stations on work surface by a robotic manipulator controlled by a computer.
